(19) United States
(12) Patent Application Publication
(10) Pub. No.: US 2023/0374563 A1
(43) Pub. Date: Nov. 23, 2023

(54) Methods for Antimicrobial Susceptibility Testing

(71) Applicants: MicrobeDX, Inc., Pacific Palisades, CA (US); The Regents of the University of California, Oakland, CA (US)

(72) Inventors: Bernard Churchill; Scott Adam Churchman, Santa Monica, CA (US); David Arnold Haake, Culver City, CA (US); Colin Wynn Halford, Los Angeles, CA (US); Roger Knauf, Cincinnati, OH (US); Gabriel Monti, Cypress, CA (US); Horacio Kido, Irvine, CA (US); Marc Madou, Irvine, CA (US); Alexandra Perebikovsky, Irvine, CA (US); Yujia Liu, Irvine, CA (US); Daniel Gussin, Encinitas, CA (US)

(21) Appl. No.: 18/227,801

(22) Filed: Jul. 28, 2023

Related U.S. Application Data
(63) Continuation of application No. 16/639,624, filed on Feb. 17, 2020.
(60) Provisional application No. 62/671,380, filed on May 14, 2018.
(60) Provisional application No. 62/552,332, filed on Aug. 30, 2017.
(60) Provisional application No. 62/547,361, filed on Aug. 18, 2017.

(30) Foreign Application Priority Data
Aug. 3, 2018 (WO) PCT/US2018/045211

(51) Int. Cl. C12Q 1/18; C12Q 1/689; G01N 33/487

(52) U.S. Cl. CPC C12Q 1/18; C12Q 1/689; G01N 33/487; C12Q 2600/106

(57) ABSTRACT

A method for determining the susceptibility of bacteria in a clinical sample comprising urine or an inoculant derived therefrom to an antibiotic agent may include the steps of a) inoculating a test portion of the clinical sample in a medium containing a predetermined concentration of the antibiotic agent; b) inoculating a control portion of the clinical sample in a medium that does not contain the antibiotic agent; c) incubating the test portion for an incubation period; d) incubating the control portion for the incubation period; e) determining a quantity of RNA in the test portion and a quantity of RNA in the control portion at the conclusion of the incubation period that is less than 480 minutes after the completion of step a); and f) determining a susceptibility of the bacteria to the antibiotic agent by comparing the quantity of RNA in the test portion to the quantity of the RNA in the control portion.

### RELATED APPLICATIONS

This application is a continuation of U.S. application Ser. No. 16/639,624, filed on Feb. 17, 2020, which is a national stage entry of PCT Application No. PCT/US2018/047075, which in turn claims the benefit under 35 U.S.C. § 119(e) of U.S. provisional patent application Ser. No. 62/547,361, filed Aug. 18, 2017 and Entitled Methods For Antimicrobial Susceptibility Testing; U.S. provisional patent application Ser. No. 62/671,380, filed May 14, 2018 and entitled Methods For Estimating Bacterial Density In Specimens By Measurement Of Ribosomal RNA; U.S. provisional patent application Ser. No. 62/552,332 filed Aug. 30, 2017 and entitled Device for Optimization of Microorganism Growth in Liquid Culture; and PCT Application No. PCT/US18/45211 filed Aug. 3, 2018 and entitled Methods for Lysis of Cells Within a Sample. The contents of these applications being incorporated herein in their entirety by reference.

### FIELD

In one of its aspects, the present invention relates to a method of determining the susceptibility of a microorganism to an antimicrobial agent, and more particularly to a method of determining of the susceptibility of a microorganism to an antimicrobial agent that combines a molecular measure of susceptibility with a predetermined concentration of antimicrobial agent.

### BACKGROUND

The analysis of biological fluid samples, particularly the detection of certain target molecules within a biological fluid, has many clinical applications. For example, the isolation and identification of uropathogens in urine samples is an important aspect of the clinical management of patients with urinary tract infections (UTIs) and other infectious diseases.

Culture-based methods for isolating and identifying uropathogens are known in the art; however, these methods can be time consuming, labor intensive, and are not cost effective. Recent advances in technology have allowed for the development of electrochemical DNA biosensors with molecular diagnostic capabilities, including bacterial pathogen detection. To run a successful electrochemical assay, a target cell can first be lysed such that a nucleic acid molecule, such as RNA, can be released from within the cell. Thus, the use of electrochemical DNA biosensors relies on the efficient lysis and release of target molecules from the cells to be diagnosed. These cells may include, among others, prokaryotic cells such as Gram-negative bacteria or Gram-positive bacteria, or fungal cells, such as yeast.

In some circumstances, a biological fluid may contain microorganisms, such as bacteria, and it may be desirable to determine if a given microorganism is susceptible to treatment by one or more antimicrobial agents. For example, if a biological fluid contains bacteria, it may be useful to determine if the particular bacteria in the sample is susceptible to, or alternatively, is resistant to, one or more antibiotics. The effectiveness of an antibiotic can vary with the resistance of a bacterial pathogen to the antibiotic. Therefore, determining the antimicrobial sensitivity of bacterial pathogens in a clinical specimen is a key step in the diagnosis and treatment of infectious diseases.

Two common methods of phenotypic antimicrobial susceptibility testing (“AST”) are broth microdilution and Kirby-Bauer disc diffusion. While such methods can be relatively accurate in determining the antimicrobial sensitivity of bacterial pathogens in clinical specimen, both are relatively slow, requiring lengthy incubation times of the sample with the antibiotics (up to 24 hours). Such methods also often require a lengthy pre-incubation culturing period (24-72 hours) to generate the AST sample, can be relatively labor-intensive, and can be challenging to automate.

Due to the relatively serious nature of infectious diseases, it can be the case that treatment should not be delayed. Therefore, antibiotic treatment is frequently started before AST results can be obtained using conventional, non-molecular, and slow-acting testing methods. This can lead to a patient being given antibiotics, or other antimicrobial agents, without first knowing if the particular bacteria afflicting the patient is susceptible or resistant to the particular antibiotic administered. If the bacteria are in fact resistant, the initial course of antibiotics may be ineffective, which may contribute to a known problem/trend of patients receiving unnecessary or less effective antibiotics when other, potentially more effective antibiotics may have been available for use. This can be particularly problematic due to the rise in drug-resistant microorganisms.

Despite the advances made to date in determining the antimicrobial sensitivity of bacterial pathogens in a clinical specimen, there is room for improvement to address the above-mentioned problems and shortcomings of the prior art.

### SUMMARY

It is an object of the present invention to obviate or mitigate at least one of the above-mentioned disadvantages of the prior art.

It is another object of the present invention to provide a novel method for determining the susceptibility of a microorganism to an antimicrobial agent.

Accordingly, in one of its aspects, the present invention provides a method for determining the susceptibility of a bacteria in a clinical sample comprising urine or an inoculant derived therefrom to an antibiotic agent, the method comprising: (a) inoculating a test portion of a clinical sample in a medium containing a predetermined concentration of an antibiotic agent; (b) inoculating a control portion of the urine sample in a medium that does not contain the antibiotic agent; (c) incubating the test portion for an incubation period; (d) incubating the control portion for the incubation period; (e) determining a quantity of RNA in the test portion and quantity of RNA in the control portion at the conclusion of the incubation period that is less than 420 minutes after the completion of step a); and (f) determining a susceptibility of the bacteria to the antibiotic agent by comparing the quantity of RNA in the test portion to the quantity of the RNA in the control portion.

In another of its aspects, the present invention provides a method of determining the susceptibility of a microorganism in a sample comprising a bodily fluid or an inoculant derived therefrom to at least two different antimicrobial agents, the method comprising the steps of: (a) inoculating a first test portion of the sample in a medium containing a first predetermined concentration of a first antimicrobial agent; (b) inoculating a second test portion of the sample in a medium containing a second a predetermined concentration of a second antimicrobial agent; (c) inoculating a control portion of the sample in a medium that does not contain either the first or second antimicrobial agents; (d) incubating the first test portion for a first incubation period, the second test portion for a second incubation period, and the control portion for a control incubation period, wherein each of the first incubation period, the second incubation period, and the control incubation period are less than 420 minutes; (e) determining a quantity of a nucleic acid molecule in the first test portion at the conclusion of the first incubation period, determining a quantity of the nucleic acid molecule in the second test portion at the conclusion of the second incubation period and determining a quantity of the nucleic acid molecule in the control portion at the conclusion of the control incubation period; (f) determining a susceptibility of the microorganism to the first antimicrobial agent by comparing the quantity of the nucleic acid molecule in the first test portion to the quantity of the quantity of the nucleic acid molecule in the control portion; and (g) determining a susceptibility of the microorganism to the second antimicrobial agent by comparing the quantity of the nucleic acid molecule in the second test portion to the quantity of the quantity of the nucleic acid molecule in the control portion.

In another of its aspects, the present invention provides a method for determining the susceptibility of a microorganism in a sample to an antimicrobial agent, the method comprising: (a) inoculating a test portion of the sample in a medium containing a predetermined concentration of an antimicrobial agent; (b) inoculating a control portion of the sample in a medium that does not contain the antimicrobial agent; (c) incubating the test portion and the control portion for an incubation period that is less than 420 minutes; (d) determining a quantity of a nucleic acid molecule in the test portion and quantity of the nucleic acid molecule in the control portion at the conclusion of the incubation; and (e) determining a susceptibility of the microorganism to the antimicrobial agent by comparing the quantity of the nucleic acid molecule in the test portion to the quantity of the quantity of the nucleic acid molecule in the control portion.

Thus, the present inventors have developed a novel method for determining the antimicrobial susceptibility of a microorganism in a clinical specimen. This method uses a molecular measure of the susceptibility of a microorganism to a given antimicrobial agent using a pre-determined, non-standard and concentration of the antimicrobial agent (as compared to the concentrations that would be used in other, non-molecular susceptibility testing procedures). When using a molecular measurement technique, the growth of a given microorganism during the test process can be determined by measuring the presence, absence, or relative concentrations of target molecular features as a proxy for growth, such as, in some of the examples described herein, nucleic acid molecules within the microorganisms.

The methods described herein may include comparing the quantity of a nucleic acid molecule from a microorganism that has not been exposed to an antimicrobial agent to the quantity of a nucleic acid molecule from a microorganism that has been exposed to an enhanced concentration of an antimicrobial agent. This method may help facilitate for a faster distinction between antimicrobial susceptible and antimicrobial resistant populations of microorganisms in a clinical specimen, as compared to the conventional AST methods.

Some methods of quantifying nucleic acid molecules in a sample, such as bacterial ribosomal RNA (“rRNA”), can generally include the steps of: 1) Lysis to release rRNA; 2) Neutralization; 3) Hybridization of target rRNA with a capture probe and detector probe; and 4) Detection of capture probe—target rRNA—detector probe complexes.

The lysing operations may be conducted using suitable lysing techniques, including those described herein. Determination of rRNA concentration may be based on a linear log-log correlation between the assay signal and rRNA analyte concentration. A synthetic target molecule at a known concentration may be included as a positive control for normalization of assay signal intensity, whereby the assay signal generated by a sample may be compared with the positive control result to determine the number of target rRNA molecules per volume tested (concentration).

It is generally known that the number of a given target nucleic acid molecule, such as the number of rRNA copies, per cell may vary widely between specimens/microorganisms. For example, rRNA copies per cell in cultivated specimens may vary from as high as approximately 100,000 copies per cell to as low as approximately 6,000 copies per cell, depending on the growth phase and density of bacteria cultivated in the growth medium. It was previously believed that such variation may make it difficult to satisfactorily determine a quantity of the microorganism based on the significantly variable number of nucleic acid molecules in a test sample. Therefore, one aspect of the teachings herein is related to a novel method for estimating bacterial or microorganism density in a specimen based on the quantity of a target nucleic acid molecule within the specimen.

As described herein, utilizing the molecular counting/quantification techniques described herein may help provide acceptably accurate results from an AST in a relatively faster time than can be achieved using conventional visual and/or microscopic inspection quantification techniques when testing similar cellular material, under similar incubation conditions, and when utilizing a similar dosage/concentration of an antimicrobial agent. However, the inventors have also discovered that the length of incubation time that is required for a given AST can be modified by changing the concentration of the antimicrobial agent that is used to a pre-determined concentration.

To the knowledge of the inventors, a method of determining the antimicrobial susceptibility of a microorganism having such a combination of features is heretofore unknown.

Other advantages of the teachings described herein may become apparent to those of skill in the art upon reviewing the present specification.

### BRIEF DESCRIPTION OF THE DRAWINGS

Embodiments of the present invention will be described with reference to the accompanying drawings, wherein like reference numerals denote like parts, and in which:

FIG. 1 depicts a graph comparing EC6210 growth in a 96-well plate, incubation disc in a shaker, and incubation disc in a new incubator by Luminex signal.

FIG. 2 depicts levels of microorganism in various samples after culture with ampicillin for 60 minutes. RiboResponse % refers to the percentage of ribosomal RNA calculated in the culture with ampicillin compared to the amount in a control lacking ampicillin.

FIG. 3 depicts levels of microorganism in various samples after culture with ampicillin for 90 minutes. RiboResponse % refers to the percentage of ribosomal RNA calculated in the culture with ampicillin compared to the amount in a control lacking ampicillin.

FIG. 4 depicts levels of microorganism in various samples after culture with cefazolin for 60 minutes. RiboResponse % refers to the percentage of ribosomal RNA calculated in the culture with cefazolin compared to the amount in a control lacking cefazolin.

FIG. 5 depicts levels of microorganism in various samples after culture with cefazolin for 90 minutes. RiboResponse % refers to the percentage of ribosomal RNA calculated in the culture with cefazolin compared to the amount in a control lacking cefazolin.

FIG. 6 depicts levels of microorganism in various samples after culture with ceftriaxone for 90 minutes. RiboResponse % refers to the percentage of ribosomal RNA calculated in the culture with ceftriaxone compared to the amount in a control lacking ceftriaxone.

FIG. 7 depicts levels of RiboResponse % over time of samples considered to be either susceptible or resistance to ceftriaxone after exposure to 32 μg/mL of ceftriaxone. RiboResponse % refers to the percentage of ribosomal RNA calculated in the culture with ceftriaxone compared to the amount in a control lacking ceftriaxone.

FIG. 8 illustrates copies of ribosomal RNA of a positive control (i.e., no antibiotic exposure) over time. Overlaid on the positive control data is theoretical examples of copies of ribosomal RNA of resistant and susceptible bacteria over time. As depicted, the curve of rRNA copies for resistant bacteria would be similar to that of the positive control for growth.

FIG. 9 is a preferred embodiment of an apparatus for use in carrying out mechanical lysis comprising a spin platform (left) and centrifugal disk (right);

FIG. 10 illustrates improved cell lysis using a combination of mechanical lysis and non-mechanical lysis;

FIG. 11 illustrates improved cell lysis using a combination of mechanical lysis and non-mechanical lysis for a broad variety of Gram-positive bacteria;

FIG. 12 illustrates optimal signal with a combination of mechanical lysis (OmniLyse®) plus NaOH for Gram-positive bacteria;

FIG. 13 illustrates improved signal with a combination of mechanical lysis (OmniLyse®) plus NaOH for a broad variety of Gram-positive bacteria;

FIG. 14 illustrates rRNA detection for various NaOH concentrations and mechanical lysis durations;

FIG. 15 illustrates Luminex signal after NaOH treatment from 0 to 5 minutes following a 1-minute mechanical lysis (OmniLyse®).

FIG. 16 illustrates a comparison of different enzyme concentrations when used in biological lysis of Gram-positive cells.

FIG. 17A illustrates a comparison of differing lengths of time of mechanical lysis (OmniLyse®) in combination with alkaline lysis.

FIG. 17B illustrates a comparison of different concentrations of NaOH in combination with mechanical lysis (OmniLyse®).

FIG. 18 illustrates the Luminex signal after lysing certain types of cells, including Gram-negative cells, Gram-positive cells, and yeast cells.

FIG. 19 illustrates the effect of different buffers used to neutralize a cell lysate.

FIG. 20, in a flowchart, illustrates the steps involved in quantifying bacterial density in a urine specimen using the rRNA concentration of bacteria in the specimen;

FIG. 21, in a graph, illustrates the correlation between rRNA concentration and density of E. coli in urine specimens from patients with urinary tract infection;

FIG. 22, in a graph, illustrates the correlation between rRNA copies per cell and density of E. coli in urine specimens from patients with urinary tract infection;

FIG. 23, in a graph, illustrates the contrast between rRNA copies per cell and density of E. coli cultivated in growth medium vs. E. coli in urine specimens from patients with urinary tract infection; and

FIG. 24, in a graph, illustrates AST assay results for Ceftriaxone when incubation was conducted on a centrifugal disc.

### DETAILED DESCRIPTION

Various apparatuses or processes will be described below to provide an example of an embodiment of each claimed invention. No embodiment described below limits any claimed invention and any claimed invention may cover processes or apparatuses that differ from those described below. The claimed inventions are not limited to apparatuses or processes having all of the features of any one apparatus or process described below or to features common to multiple or all of the apparatuses described below. It is possible that an apparatus or process described below is not an embodiment of any claimed invention. Any invention disclosed in an apparatus or process described below that is not claimed in this document may be the subject matter of another protective instrument, for example, a continuing patent application, and the applicants, inventors, or owners do not intend to abandon, disclaim, or dedicate to the public any such invention by its disclosure in this document.

Conventional methods for determining the bacterial (microbial) density in a sample (whether as a standalone process or part of a multi-stage assay such as an AST) often require at least one growth phase, in which an enriched bacterial culture is prepared from the specimen. Such methods may be relatively accurate but may tend to be relatively slow, taking several hours, days, or weeks to provide useful results. In addition to the time required for determining the microbial density, conventional AST methods for determining the susceptibility of a microorganism to an antimicrobial agent in a sample may be relatively accurate but tend to be relatively slow, taking several hours or days to complete. In a clinical environment, such time frames may be undesirable and may be considered too long a time period to withhold/delay treatment for a subject. This time delay can sometimes lead to treatments being implemented, such as a particular antibiotic being prescribed before the AST results are obtained. This may lead to the unnecessary prescription of antibiotics and/or the prescription of an antibiotic that is less effective in treating a particular infection than other available antibiotics. In some circumstances, time may be of the essence when determining the susceptibility of a bacteria, or other microorganism, to an antimicrobial agent.

For example, a given clinical specimen may be obtained from a subject with a suspected infection who may require further medical treatment based on the results of the analysis of the clinical specimen. For example, urine specimens are often obtained from subjects experiencing symptoms consistent with urinary tract infections. In these circumstances, it may be desirable to analyze the specimen's response to a variety of different antibiotic agents that could possibly be prescribed to the subject and to determine which of such agents is likely to be relatively more or less effective than the others. For convenience, such analysis would preferably be conducted in a relatively short time period, such as during a routine doctor's visit or in a period of time that the subject might be reasonably expected to wait at the testing location. Preferably, this time period may be less than about 4 hours (or other time limits mentioned herein), and more preferably may be less than about 90 minutes or less than about 60 minutes. This may help a clinician obtain the results while the subject/patient waits, and to then prescribe a desired antibiotic agent for treatment.

Optionally, a particular clinical sample may be tested with respect to two or more antimicrobial agents simultaneously. For example, a clinical sample may be sub-divided into two or more test portions, along with at least one control portion, that can be separately, but simultaneously tested. In some arrangements, a clinical sample may be sub-divided into seven test portions and one control portion, with each test portion being exposed to a different antimicrobial agent during their respective incubation periods and then being evaluated with respect to a common control portion.

Preferably, tests that are being conducted in parallel may be configured so that the respective incubation periods for each of the test portions are approximately equal, whereby each of the test portions can be processed/quantified at about the same time. This may also help facilitate the use of a common control portion, as compared to operating tests with different incubation periods which may preferably be compared to different, respective control portions having substantially the same incubation period. Configuring each of the test and control incubation portions to be about the same, such as each being about 90 minutes or about 60 minutes, may help reduce the need for an operator or technician to monitor the tests at different time intervals, and may allow an operator to initiate all of the tests and then only need to return to collect the results at the end of the pre-set incubation period (i.e., set a machine to perform the tests and only have to return after 60 or 90 minutes have passed, rather than having to return at different times to observe the results of the different tests).

In some circumstances, the variety of different antimicrobial agents to be tested may have incubation periods that are sufficiently similar under the expected testing conditions and using conventional concentration/dosages. In other circumstances, utilizing conventional concentrations/dosages of the antimicrobial agents may lead to incubation times that are different, and do not lend themselves to being processed/quantified at the same time and/or being compared to a common control portion. To help facilitate the parallel/simultaneous testing of different antimicrobials, the inventors have discovered that modifying the concentration/dosage of a given antimicrobial agent can affect the length of its associated incubation period, under otherwise similar conditions. For example, the inventors have discovered, as described herein, that a given antimicrobial can be provided in a predetermined concentration that can help provide an incubation period having a targeted length of time—such as about 90 minutes or about 60 minutes. It has also been discovered that the predetermined concentration that is used to provide an incubation period of about 90 minutes, for example, may be different for different antimicrobial agents. In such cases, each antimicrobial agent may be provided in a different, predetermined concentration such that each of the tests to be conducted can each have approximately the same incubation period. In these examples, the target parameter that is to be achieved is a desired incubation time that can be synchronized with the incubation times for other tests being conducted in parallel. In some other examples, instead of configuring the incubation period to have a target duration/length, the predetermined, concentration could be selected to target another parameter, such as configuring an AST to provide useful results in the shortest possible time frame, combined with the molecular analysis techniques, or to configuring an AST to provide useful results while consuming a relatively small amount of the particular antimicrobial agent (regardless of the incubation time), or a balance of all of these factors.

For example, a blood sample may be obtained from a patient experiencing symptoms consistent sepsis or other bloodborne, microorganism-based conditions. In such circumstances, completing a suitable accurate AST in the shortest practical time may be desirable, even if the testing of different antimicrobial agents requires different incubation periods. Treatment for the patient could then begin once the first acceptable antimicrobial agent has been identified, rather than waiting until the end of the longest of the incubation periods. Such tests may be likely to be performed in hospitals or other such environments, where sufficient staff can be available to conduct and monitor a variety of tests in parallel. In other examples, an apparatus for conducting such tests may be configured to automatically read the results from each separate test at different times. To help facilitate this approach, additional control portions can be used, and preferably, at least one control portion can be provided for each test portion to be analyzed (i.e., pairs of corresponding test and control portions can be provided). As each test portion reaches the end of its incubation period, it can be processed and compared to the condition of its respective control sample, as described herein. In these examples, predetermined concentration can be the concentration that provides the shortest incubation period without compromising the accuracy of the test results. For a given antimicrobial agent, this may be different than the predetermined concentration used when configuring the incubation period to have a target duration.

In some other situations, it may be desirable to obtain useful test results while minimizing the amount of the antimicrobial agent consumed during the testing process. This may be desirable if the antimicrobial agent is in relatively short supply and/or is relatively expensive. In such examples, the predetermined concentration may be the minimal amount of a given antimicrobial agent that is sufficient to obtain useful, and acceptably accurate test results. This concentration may be different than the concentration in the other examples described herein.

In general, the performance and associated speed of performing the methods described herein can be related to techniques and methods used for the incubating, lysing, and quantifying the test specimens along with the predetermined concentration(s) of the antimicrobial agents used. The particular predetermined, concentration for a given antimicrobial to be used in a given circumstance (e.g. when trying to achieve a particular objective or effect on the incubation period) may be selected based on the nature of the test being conducted, whether the test is being conducted alone or in combination with the testing of other antimicrobial agents, the urgency of the test results, and other such factors.

Preferably, an apparatus, such as a test cartridge or centrifugal disc can be pre-loaded with a predetermined, concentration of a given antimicrobial and then made available to a clinic or user in a corresponding use circumstance. For example, an eight-channel centrifugal disc can have one control channel and can have its other channels pre-loaded with seven different antimicrobial agents in, potentially different, predetermined concentrations so that all of the test channels have an incubation period of about 60 minutes. The particular antimicrobial agents used can be pre-selected to be those that are available in a given region or that are, based on past experience, relatively likely to be effective against the types of microbes that may be expected for a given test. For example, a UTI assessment disc could be pre-loaded with the seven antimicrobial agents that may be expected to be effective in treating the types of bacteria that may be expected to be present in a clinical urine sample. Such discs could be stocked in doctors' offices, clinics, and other such locations where patients may seek medical attention.

Furthermore, conventional quantification and AST techniques may require a skilled technician to set-up and run the bacterial cultures, as well as to interpret the results. The analysis may also require specialized and/or costly equipment. As such equipment and skilled technicians can be relatively scarce resources, they are often located in centralized labs and/or hospital environments which are removed from common frontline care facilities, such as a physician's or veterinarian's office, walk-in clinics, and the like. This arrangement can further delay the processing and analysis of clinical specimens by several hours or days, as the specimens must be physically transported from the front-line environment to a centralized testing location and may then wait in a testing queue or backlog of samples awaiting analysis. This time-delay may reduce the accuracy of the ensuing clinical specimen analysis due to such factors as growth or death of any bacteria that may be present in the specimen.

Therefore, there remains a need for synchronizing the incubation times for different antimicrobial compounds to help perform multiple different tests simultaneously, reducing the amount of a given antimicrobial agent required to obtain an accurate AST test result. There also remains a need for relatively faster specimen analysis methods, and a need to be able to perform at least some of the analysis in situ in a front-line setting, such as in a physician's or veterinarian's office, instead of having to physically transport the specimens to a centralized location. Similarly, it would be advantageous to provide a method in which a clinically meaningful test result (i.e., information that can help inform treatment decisions) can be provided to a caregiver without requiring the individual skill and judgment of a skilled technician.

To help mitigate at least some of these deficiencies in conventional methods of specimen analysis, the present inventors have developed the process and methods described herein, including a method in which it may be possible to estimate the microorganism density and susceptibility to an antimicrobial agent in a specimen in situ, in a front line setting, and in less time than conventional methods may allow for. In contrast to the established practices of determining the susceptibility of a microorganism to an antimicrobial agent, the present inventors have discovered a method, which combines a molecular measure of antimicrobial susceptibility with a predetermined concentration of antimicrobial agent, that may provide a faster distinction between antimicrobial susceptible and antimicrobial resistant populations of microorganisms in a clinical specimen, as compared to conventional AST methods.

In addition to reducing the time required to perform AST on a clinical specimen, it may be desirable to determine the susceptibility of the microorganisms in a specimen to multiple antimicrobial agents to ensure treatment includes the most appropriate antibiotic or combination of antibiotics. It may be further desirable to test such susceptibility to multiple antimicrobials simultaneously/in parallel, thereby streamlining the AST process by providing a single test in which the response to multiple antimicrobials can be compared to a common control. As such, the present inventors have developed a method in which it may be possible to estimate microorganism density and susceptibility to multiple antimicrobial agents in a specimen in less time than conventional methods may allow and utilizing a common incubation period duration.

Disclosed herein are methods for determining the susceptibility of a microorganism to one or more antimicrobial agents. Determining the susceptibility of a microorganism to an antimicrobial agent may comprise comparing the quantity of a nucleic acid molecule from a microorganism that has not been exposed to an antimicrobial agent to the quantity of a nucleic acid molecule from a microorganism that has been exposed to a predetermined concentration of an antimicrobial agent. Use of the predetermined concentrations of antimicrobial agents in the methods disclosed herein may allow for faster antimicrobial susceptibility testing.

In accordance with one broad aspect of the teachings described herein, a method for determining the susceptibility of bacteria in a clinical sample comprising urine or an inoculant derived therefrom to an antibiotic agent, the method comprising: (a) inoculating a test portion of the clinical sample in a medium containing a predetermined concentration of the antibiotic agent; (b) inoculating a control portion of the clinical sample in a medium that does not contain the antibiotic agent; (c) incubating the test portion for an incubation period; (d) incubating the control portion for the incubation period; (e) determining a quantity of RNA in the test portion and a quantity of RNA in the control portion at the conclusion of the incubation period that is less than 420 minutes after the completion of step a); and (f) determining a susceptibility of the bacteria to the antibiotic agent by comparing the quantity of RNA in the test portion to the quantity of the RNA in the control portion.

Preferred embodiments of this method may include any one or a combination of any two or more of any of the following features:
incubating the test portion is done within a test incubation chamber on a centrifugal disc, and incubating the control portion is done within a control incubation chamber on the same centrifugal disc;
the test incubation chamber is fluidically isolated from the control incubation chamber; the RNA comprises pre-ribosomal RNA;
the RNA comprises mature RNA;
the RNA comprises ribosomal RNA;
the RNA comprises 16S rRNA;
the RNA comprises 23S rRNA;
the incubation period is equal to or less than 450 minutes;
the incubation period is equal to or less than 420 minutes;
the incubation period is equal to or less than 390 minutes;
the incubation period is equal to or less than 360 minutes;
the incubation period is equal to or less than 300 minutes;
the incubation period is equal to or less than 270 minutes;
the incubation period is equal to or less than 240 minutes;
the incubation period is equal to or less than 210 minutes;
the incubation period is equal to or less than 150 minutes;
the incubation period is equal to or less than 120 minutes;
the incubation period is equal to or less than 90 minutes;
the incubation period is equal to or less than 60 minutes;
the incubation period is equal to or less than 30 minutes;
the antibiotic agent is a bactericidal antibiotic;
the antibiotic agent is a bacteriostatic antibiotic;
the antibiotic agent comprises at least one of Gentamicin, Ciprofloxacin, Cefazolin, Ceftriaxone, Cefepime, Ampicillin, Trimethoprim-Sulfamethoxazole, Nitrofurantoin, Fosfomycin, Amoxicillin-Clavulanate, Amikacin, Ertapenem, Meropenem and combinations thereof;
the predetermined concentration is above the sensitive CLSI MIC cutoff (for urine) for the antibiotic agent;
the predetermined concentration is above the intermediate CLSI MIC cutoff (for urine) for the antibiotic agent;
the predetermined concentration is above the resistant CLSI MIC cutoff (for urine) for the antibiotic agent;
the predetermined concentration is at least 2-fold or greater than the resistant CLSI MIC cutoff (for urine) for the antibiotic agent;
the predetermined concentration is at least 4-fold or greater than the resistant CLSI MIC cutoff (for urine) for the antibiotic agent;
the predetermined concentration is between the intermediate CLSI MIC cutoff and the resistant CLSI MIC cutoff (for urine) for the antibiotic agent;
the predetermined concentration is below the sensitive CLSI MIC cutoff (for urine) for the antibiotic agent;
the sensitive CLSI MIC cutoff (for urine) is at least 2-fold or greater than the predetermined concentration for the antibiotic agent;
the antibiotic agent comprises Gentamicin and the predetermined concentration is between about 2 μg/mL and 16 μg/mL;
the predetermined concentration is between about 2 μg/mL and 4 μg/mL;
the predetermined concentration is about 2 μg/mL;
the predetermined concentration is about 4 μg/mL;
the sensitive CLSI MIC cutoff (for urine) for Gentamicin is equal to or greater than the predetermined concentration;
the antibiotic agent comprises Ciprofloxacin and the predetermined concentration is between about 1 μg/mL and 8 μg/mL;
the predetermined concentration is between about 1 μg/mL and 4 μg/mL;
the predetermined concentration is about 4 μg/mL;
the predetermined concentration is substantially equal to the resistant CLSI MIC cutoff (for urine) for Ciprofloxacin;
the antibiotic agent comprises Cefazolin and the predetermined concentration is between about 2 μg/mL and about 256 μg/mL;
the predetermined concentration is between about 16 μg/mL and about 128 μg/mL;
the predetermined concentration is about 64 μg/mL;
the predetermined concentration is substantially equal to 2 times the resistant CLSI MIC cutoff (for urine) for Cefazolin;
the antibiotic agent comprises Ceftriaxone and the predetermined concentration is between about 1 μg/mL and about 128 μg/mL;
the predetermined concentration is between about 16 μg/mL and about 64 μg/mL;
the predetermined concentration is about 32 μg/mL;
the predetermined concentration is substantially equal to 8 times the resistant CLSI MIC cutoff (for urine) for Ceftriaxone;
the antibiotic agent comprises Cefepime and the predetermined concentration is between about 4 μg/mL and about 128 μg/mL;
the predetermined concentration is between about 16 μg/mL and about 128 μg/mL;
the predetermined concentration is between about 32 μg/mL and about 64 μg/mL;
the predetermined concentration is about 32 μg/mL;
the predetermined concentration is about 64 μg/mL;
the predetermined concentration is substantially equal to 2 or 4 times the resistant CLSI MIC cutoff (for urine) for Cefepime;
the antibiotic agent comprises Ampicillin and the predetermined concentration is between about 8 μg/mL and about 2048 μg/mL;
the predetermined concentration is between about 128 μg/mL and about 512 μg/mL;
the predetermined concentration is about 128 μg/mL;
the predetermined concentration is about 512 μg/mL;
the predetermined concentration is substantially equal to about 4 times the resistant CLSI MIC cutoff (for urine) for Ampicillin;
the predetermined concentration is substantially equal to about 16 times the resistant CLSI MIC cutoff (for urine) for Ampicillin;
the antibiotic agent comprises Trimethoprim-Sulfamethoxazole and the predetermined concentration for Trimethoprim is between about 2 μg/mL and about 16 μg/mL and the predetermined concentration for Sulfamethoxazole is between about 38 μg/mL and about 304 μg/mL;
the predetermined concentration for Trimethoprim is between about 4 μg/mL and about 8 μg/mL and the predetermined concentration for Sulfamethoxazole is between about 76 μg/mL and about 152 μg/mL;
the predetermined concentration for Trimethoprim is about 4 μg/mL and the predetermined concentration for Sulfamethoxazole is about 76 μg/mL;
the predetermined concentration for Trimethoprim-Sulfamethoxazole is substantially equal to the resistant CLSI MIC cutoff (for urine) for Trimethoprim-Sulfamethoxazole; the antibiotic agent comprises Nitrofurantoin and the predetermined concentration is between about 4 μg/mL and about 512 μg/mL;
the predetermined concentration is between about 8 μg/mL and about 32 μg/mL;
the predetermined concentration is about 16 μg/mL;
the sensitive CLSI MIC cutoff (for urine) for Nitrofurantoin is at least 2-fold or greater than the predetermined concentration;
the antibiotic agent comprises Fosfomycin and the predetermined concentration is between about 4 μg/mL and about 512 μg/mL;
the predetermined concentration is between about 8 μg/mL and about 128 μg/mL;
the predetermined concentration is about 64 μg/mL;
the sensitive CLSI MIC cutoff (for urine) for Fosfomycin is at about equal to the predetermined concentration;
the antibiotic agent comprises Amoxicillin-Clavulanate and the predetermined concentration for Amoxicillin is between about 2 μg/mL and about 256 μg/mL and the predetermined concentration for Clavulanate is between about 1 μg/mL and about 128 μg/mL;
the predetermined concentration for Amoxicillin is between about 8 μg/mL and about 128 μg/mL and the predetermined concentration for Clavulanate is between about 4 μg/mL and about 64 μg/mL;
the predetermined concentration for Amoxicillin is about 64 μg/mL and the predetermined concentration for Clavulanate is about 32 μg/mL;
the predetermined concentration for Amoxicillin is about 32 μg/mL and the predetermined concentration for Clavulanate is about 16 μg/mL;
the predetermined concentration for Amoxicillin is about 16 μg/mL and the predetermined concentration for Clavulanate is about 8 μg/mL;
the predetermined concentration is equal to the intermediate CLSI MIC cutoff (for urine) for Amoxicillin-Clavulanate;
the predetermined concentration is greater than the intermediate CLSI MIC cutoff (for urine) for Amoxicillin-Clavulanate;
the predetermined concentration is equal to or greater than the resistant CLSI MIC cutoff (for urine) for Amoxicillin-Clavulanate;
the antibiotic agent comprises Amikacin and the predetermined concentration is between about 2 μg/mL and about 64 μg/mL;
the predetermined concentration is between about 8 μg/mL and about 64 μg/mL;
the predetermined concentration is about 32 μg/mL;
wherein the predetermined concentration is about 16 μg/mL;
the predetermined concentration is about 8 μg/mL;
the predetermined concentration is less than the resistant CLSI MIC cutoff (for urine) for Amikacin;
the predetermined concentration is equal to the intermediate CLSI MIC cutoff (for urine) for Amikacin;
the predetermined concentration is less than or equal to the sensitive CLSI MIC cutoff (for urine) for Amikacin;
the antibiotic agent comprises Ertapenem and the predetermined concentration is between about 0;5 μg/mL and about 8 μg/mL;
the predetermined concentration is between about 1 μg/mL and about 4 μg/mL;
the predetermined concentration is about 4 μg/mL;
the predetermined concentration is about 2 μg/mL;
the predetermined concentration is greater than or equal to the resistant CLSI MIC cutoff (for urine) for Ertapenem;
the antibiotic agent comprises Meropenem and the predetermined concentration is between about 1 μg/mL and about 8 μg/mL;
the predetermined concentration is between about 1 μg/mL and about 4 μg/mL;
the predetermined concentration is about 4 μg/mL;
the predetermined concentration is about 2 μg/mL;
the predetermined concentration is equal to the resistant CLSI MIC cutoff (for urine) for Meropenem;
the predetermined concentration is equal to the intermediate CLSI MIC cutoff (for urine) for Meropenem;
determining a baseline quantity of RNA in the control portion before the incubation period is complete and comparing the baseline quantity of RNA to the quantity of RNA in the control portion at the end of the incubation period to determine if the quantity of RNA in the control portion increased by a measurement threshold amount during the incubation period;
the bacteria comprises a Gram-negative bacterium;
the bacteria comprises a Gram-positive bacteria;
the bacteria is an unknown bacteria when steps a) to f) are conducted;
lysing the test portion prior to determining the quantity of RNA in the test portion; further comprising the steps of:
   g) subjecting the test portion to mechanical lysis to cause disruption of a cellular membrane in the bacteria;
   h) contacting the test portion with an alkaline material to produce a lysate composition comprising the RNA; and
   i) recovering the lysate composition from the test portion;
Step h) comprises contacting the bacteria in the test portion with an alkaline liquid;
Step h) comprises contacting the bacteria in the test portion with an alkaline solution;
the alkaline solution is a sodium hydroxide solution;
the alkaline solution has a concentration of 10 M or less;
the alkaline solution has a concentration in the range of from 1 M to 5M;
the alkaline solution has a concentration in the range of from 1;5 M to 3M;
the alkaline solution has a concentration of 2M;
the alkaline solution has a concentration of 3M;
lysing the test portion comprises transferring an aliquot of an inoculate to a lysing container;
incubating the test portion is done within a test incubation chamber on a centrifugal disc, and lysing the test portion is conducted within a lysing chamber on the same centrifugal disc;
the lysing chamber is fluidically connected to the test incubation chamber;
the lysing chamber comprises the test incubation chamber;
Steps g) and h) are conducted for a period of 10 minutes or less;
Steps g) and h) are conducted for a period of from 30 seconds to 10 minutes;
Steps g) and h) are conducted for a period of from 1 minute to 8 minutes;
Steps g) and h) are conducted for a period of from 2 minutes±30 seconds;
Steps g) and h) are conducted for a period of from 3 minutes±30 seconds;
Steps g) and h) are conducted for a period of from 4 minutes±30 seconds;
Steps g) and h) are conducted for a period of from 5 minutes±30 seconds;
Steps g) and h) are conducted for a period of from 6 minutes±30 seconds;
Steps g) and h) are conducted for a period of from 7 minutes±30 seconds;
Steps g) and h) are carried out concurrently;
the mechanical lysis comprises a combination of centrifugation and puck lysing;
the mechanical lysis comprises a combination of centrifugation and magnetic puck lysing;
the combination of centrifugation and puck lysing is carried out in a common lysis chamber;
Steps h) and i) are carried out concurrently;
Steps h) and i) are carried out sequentially;
Step i) is carried out after commencement of disruption of the cellular membrane in Step h);
the bacteria are susceptible to the antibiotic agent if the quantity of RNA in the control portion is more than the quantity of RNA in the test portion at the conclusion of the incubation period;
the bacteria are not susceptible to the antibiotic agent if the quantity of RNA in the control portion is nearly equal, equal, or less than the quantity of RNA in the test portion at the conclusion of the incubation period;
the microorganism is susceptible to the antibiotic agent when the quantity of RNA in the test portion is about 40% or less of the quantity of RNA in the control portion at the conclusion of the incubation period; and
the microorganism is resistant to the antibiotic agent when the quantity of RNA in the test portion is about 60% or more of the quantity of RNA in the control portion at the conclusion of the incubation period.

In another of its aspects, the present invention relates to a method of determining the susceptibility of a microorganism in a sample comprising a bodily fluid or an inoculant derived therefrom to at least two different antimicrobial agents, the method comprising the steps of: (a) inoculating a first test portion of the sample in a medium containing a first predetermined concentration of a first antimicrobial agent; (b) inoculating a second test portion of the sample in a medium containing a second a predetermined concentration of a second antimicrobial agent; (c) inoculating a control portion of the sample in a medium that does not contain either the first or second antimicrobial agents; (d) incubating the first test portion for a first incubation period, the second test portion for a second incubation period, and the control portion for a control incubation period, wherein each of the first incubation period, the second incubation period, and the control incubation period are less than 420 minutes; (e) determining a quantity of a nucleic acid molecule in the first test portion at the conclusion of the first incubation period, determining a quantity of a nucleic acid molecule in the second test portion at the conclusion of the second incubation period and determining a quantity of a nucleic acid molecule in the control portion at the conclusion of the incubation period; (f) determining a susceptibility of the microorganism to the first antimicrobial agent by comparing the quantity of the nucleic acid molecule in the first test portion to the quantity of the nucleic acid molecule in the control portion; and (g) determining a susceptibility of the microorganism to the second antimicrobial agent by comparing the quantity of the nucleic acid molecule in the second test portion to the quantity of the nucleic acid molecule in the control portion.

Preferred embodiments of this method may include any one or a combination of any two or more of any of the following features:
the first incubation period is the same as the second incubation period;
at least one of the first incubation period and the second incubation period is the same as the control incubation period;
at least one of the first incubation period and the second incubation period is less than the control incubation period
the first predetermined concentration and the second predetermined concentration are different and are configured so that the steps of determining the quantity of the nucleic acid molecule in the first test portion at the conclusion of the first incubation period and determining the quantity of the nucleic acid molecule in the second test portion are performable simultaneously;
the first incubation period is equal to or less than 420 minutes;
the first incubation period is equal to or less than 390 minutes;
the first incubation period is equal to or less than 360 minutes;
the first incubation period is equal to or less than 300 minutes;
the first incubation period is equal to or less than 270 minutes;
the first incubation period is equal to or less than 240 minutes;
the first incubation period is equal to or less than 210 minutes;
the first incubation period is equal to or less than 150 minutes;
the first incubation period is equal to or less than 120 minutes;
the first incubation period is equal to or less than 90 minutes;
the first predetermined concentration and the second predetermined concentration are different and are configured so that the first incubation period and the second incubation period are substantially the same and are both equal to or less than 90 minutes;
the first predetermined concentration and the second predetermined concentration are different and are configured so that the first incubation period and the second incubation period are substantially the same and are both equal to or less than 120 minutes;
the first incubation period is equal to or less than 60 minutes;
the first predetermined concentration and second predetermined concentration are different and are configured so that the first incubation period and the second incubation period are substantially the same and are both equal to or less than 60 minutes;
the first incubation period is equal to or less than 30 minutes;
when the first predetermined concentration and second predetermined concentration are the same but the first incubation period and the second incubation period are different;
the first predetermined concentration is different than the second predetermined concentration;
the first antimicrobial agent comprises a first antibiotic agent and the second antimicrobial agent comprises a second antibiotic agent;
the antibiotic agent is a bactericidal antibiotic;
the antibiotic agent is a bacteriostatic antibiotic;
the antibiotic agent comprises at least one of Gentamicin, Ciprofloxacin, Cefazolin, Ceftriaxone, Cefepime, Ampicillin, Trimethoprim-Sulfamethoxazole, Nitrofurantoin, Fosfomycin, Amoxicillin-Clavulanate, Amikacin, Ertapenem, Meropenem and combinations thereof;
the predetermined concentration is above the sensitive CLSI MIC cutoff (for urine) for the antibiotic agent;
the predetermined concentration is above the intermediate CLSI MIC cutoff (for urine) for the antibiotic agent;
the predetermined concentration is above the resistant CLSI MIC cutoff (for urine) for the antibiotic agent;
the predetermined concentration is at least 2-fold or greater than the resistant CLSI MIC cutoff (for urine) for the antibiotic agent;
the predetermined concentration is at least 4-fold or greater than the resistant CLSI MIC cutoff (for urine) for the antibiotic agent;
the predetermined concentration is between the intermediate CLSI MIC cutoff and the resistant CLSI MIC cutoff (for urine) for the antibiotic agent;
the predetermined concentration is below the sensitive CLSI MIC cutoff (for urine) for the antibiotic agent;
the sensitive CLSI MIC cutoff (for urine) is at least 2-fold or greater than the predetermined concentration for the antibiotic agent;
the antibiotic agent comprises Gentamicin and the predetermined concentration is between about 2 μg/mL and 16 μg/mL;
the predetermined concentration is between about 2 μg/mL and 4 μg/mL;
the predetermined concentration is about 2 μg/mL;
the predetermined concentration is about 4 μg/mL;
the sensitive CLSI MIC cutoff (for urine) for Gentamicin is equal to or greater than the predetermined concentration;
the antibiotic agent comprises Ciprofloxacin and the predetermined concentration is between about 1 μg/mL and 8 μg/mL;
The method of claim 178, wherein the predetermined concentration is between about 1 μg/mL and 4 μg/mL;
the predetermined concentration is about 4 μg/mL;
the predetermined concentration is substantially equal to the resistant CLSI MIC cutoff (for urine) for Ciprofloxacin;
the antibiotic agent comprises Cefazolin and the predetermined concentration is between about 2 μg/mL and about 256 μg/mL;
the predetermined concentration is between about 16 μg/mL and about 128 μg/mL;
the predetermined concentration is about 64 μg/mL;
the predetermined concentration is substantially equal to 2 times the resistant CLSI MIC cutoff (for urine) for Cefazolin;
the antibiotic agent comprises Ceftriaxone and the predetermined concentration is between about 1 μg/mL and about 128 μg/mL;
the predetermined concentration is between about 16 μg/mL and about 64 μg/mL;
the predetermined concentration is about 32 μg/mL;
the predetermined concentration is substantially equal to 8 times the resistant CLSI MIC cutoff (for urine) for Ceftriaxone;
the antibiotic agent comprises Cefepime and the predetermined concentration is between about 4 μg/mL and about 128 μg/mL;
the predetermined concentration is between about 16 μg/mL and about 128 μg/mL;
the predetermined concentration is between about 32 μg/mL and about 64 μg/mL;
the predetermined concentration is about 32 μg/mL;
the predetermined concentration is about 64 μg/mL;
the predetermined concentration is substantially equal to 2 or 4 times the resistant CLSI MIC cutoff (for urine) for Cefepime;
the antibiotic agent comprises Ampicillin and the predetermined concentration is between about 8 μg/mL and about 2048 μg/mL;
the predetermined concentration is between about 128 μg/mL and about 512 μg/mL;
the predetermined concentration is about 128 μg/mL;
the predetermined concentration is about 512 μg/mL;
the predetermined concentration is substantially equal to about 4 times the resistant CLSI MIC cutoff (for urine) for Ampicillin;
the predetermined concentration is substantially equal to about 16 times the resistant CLSI MIC cutoff (for urine) for Ampicillin;
the antibiotic agent comprises Trimethoprim-Sulfamethoxazole and the predetermined concentration for Trimethoprim is between about 2 μg/mL and about 16 μg/mL and the predetermined concentration for Sulfamethoxazole is between about 38 μg/mL and about 304 μg/mL;
the predetermined concentration for Trimethoprim is between about 4 μg/mL and about 8 μg/mL and the predetermined concentration for Sulfamethoxazole is between about 76 μg/mL and about 152 μg/mL;
the predetermined concentration for Trimethoprim is about 4 μg/mL and the predetermined concentration for Sulfamethoxazole is about 76 μg/mL;
the predetermined concentration for Trimethoprim-Sulfamethoxazole is substantially equal to the resistant CLSI MIC cutoff (for urine) for Trimethoprim-Sulfamethoxazole;
the antibiotic agent comprises Nitrofurantoin and the predetermined concentration is between about 4 μg/mL and about 512 μg/mL;
the predetermined concentration is between about 8 μg/mL and about 32 μg/mL;
the predetermined concentration is about 16 μg/mL;
the sensitive CLSI MIC cutoff (for urine) for Nitrofurantoin is at least 2-fold or greater than the predetermined concentration;
the antibiotic agent comprises Fosfomycin and the predetermined concentration is between about 4 μg/mL and about 512 μg/mL;
the predetermined concentration is between about 8 μg/mL and about 128 μg/mL;
the predetermined concentration is about 64 μg/mL;
the sensitive CLSI MIC cutoff (for urine) for Fosfomycin is at about equal to the predetermined concentration;
the antibiotic agent comprises Amoxicillin-Clavulanate and the predetermined concentration for Amoxicillin is between about 2 μg/mL and about 256 μg/mL and the predetermined concentration for Clavulanate is between about 1 μg/mL and about 128 μg/mL;
the predetermined concentration for Amoxicillin is between about 8 μg/mL and about 128 μg/mL and the predetermined concentration for Clavulanate is between about 4 μg/mL and about 64 μg/mL;
the predetermined concentration for Amoxicillin is about 64 μg/mL and the predetermined concentration for Clavulanate is about 32 μg/mL;
wherein the predetermined concentration for Amoxicillin is about 32 μg/mL and the predetermined concentration for Clavulanate is about 16 μg/mL;
the predetermined concentration for Amoxicillin is about 16 μg/mL and the predetermined concentration for Clavulanate is about 8 μg/mL;
the predetermined concentration is equal to the intermediate CLSI MIC cutoff (for urine) for Amoxicillin-Clavulanate;
the predetermined concentration is greater than the intermediate CLSI MIC cutoff (for urine) for Amoxicillin-Clavulanate;
the predetermined concentration is equal to or greater than the resistant CLSI MIC cutoff (for urine) for Amoxicillin-Clavulanate;
the antibiotic agent comprises Amikacin and the predetermined concentration is between about 2 μg/mL and about 64 μg/mL;
the predetermined concentration is between about 8 μg/mL and about 64 μg/mL;
the predetermined concentration is about 32 μg/mL;
the predetermined concentration is about 16 μg/mL;
the predetermined concentration is about 8 μg/mL;
the predetermined concentration is less than the resistant CLSI MIC cutoff (for urine) for Amikacin;
the predetermined concentration is equal to the intermediate CLSI MIC cutoff (for urine) for Amikacin;
the predetermined concentration is less than or equal to the sensitive CLSI MIC cutoff (for urine) for Amikacin;
the antibiotic agent comprises Ertapenem and the predetermined concentration is between about 0;5 μg/mL and about 8 μg/mL;
the predetermined concentration is between about 1 μg/mL and about 4 μg/mL;
the predetermined concentration is about 4 μg/mL;
the predetermined concentration is about 2 μg/mL;
the predetermined concentration is greater than or equal to the resistant CLSI MIC cutoff (for urine) for Ertapenem;
the antibiotic agent comprises Meropenem and the predetermined concentration is between about 1 μg/mL and about 8 μg/mL;
the predetermined concentration is between about 1 μg/mL and about 4 μg/mL;
the predetermined concentration is about 4 μg/mL;
the predetermined concentration is about 2 μg/mL;
the predetermined concentration is equal to the resistant CLSI MIC cutoff (for urine) for Meropenem;
the predetermined concentration is equal to the intermediate CLSI MIC cutoff (for urine) for Meropenem;
determining a baseline quantity of the nucleic acid molecule in the control portion before the control incubation period is complete and comparing the baseline quantity of the nucleic acid molecule to the quantity of the nucleic acid molecule in the control portion at the conclusion of the incubation period to determine if the quantity of the nucleic acid in the control portion increased by a measurement threshold amount during the incubation period;
the microorganism comprises a Gram-negative bacterium;
the microorganism comprises a Gram-positive bacterium;
the microorganism is an unknown bacterium when steps a) to f) of claim 139 are conducted; and
lysing the first test portion prior to determining the quantity of the nucleic acid in the first test portion and lysing the second test portion prior to determining the quantity of the nucleic acid in the second test portion.
further comprising the steps of:
   h) subjecting the first test portion and the second test portion to mechanical lysis to cause disruption of a cellular membrane in the microorganism in each;
   i) contacting the first test portion and the second test portion with an alkaline material to produce a first lysate composition comprising the nucleic acid in the first test portion and a second lysate composition comprising the nucleic acid in the second test portion; and
   j) recovering the first test portion lysate composition from the first test portion and the second test portion lysate composition from the second test portion.
Step i) comprises contacting the microorganisms in the first and second test portions with an alkaline liquid;
Step i) comprises contacting the microorganisms in the first and second test portions with an alkaline solution;
the alkaline solution is a sodium hydroxide solution;
the alkaline solution has a concentration of 10 M or less;
the alkaline solution has a concentration in the range of from 1 M to 5M;
the alkaline solution has a concentration in the range of from 1;5 M to 3M;
the alkaline solution has a concentration of 2M;
the alkaline solution has a concentration of 3M;
lysing the first and second test portions comprises transferring an aliquot of an inoculate from each of the first and second test portion to a first and second lysing container;
incubating the first and second test portions is done within a first and second test incubation chamber on a centrifugal disc, and lysing the first and second test portions is conducted within a first and second lysing chamber on the same centrifugal disc;
the first lysing chambers is fluidly connected to the first test incubation chamber and the second lysing chambers is fluidly connected to the second test incubation chamber;
the first lysing chamber comprises the first test incubation chamber and the second lysing chamber comprises the second test chamber;
Steps h) and i) are conducted for a period of 10 minutes or less;
Steps h) and i) are conducted for a period of from 30 seconds to 10 minutes;
Steps h) and i) are conducted for a period of from 1 minute to 8 minutes;
Steps h) and i) are conducted for a period of from 2 minutes±30 seconds;
Steps h) and i) are conducted for a period of from 3 minutes±30 seconds;
Steps h) and i) are conducted for a period of from 4 minutes±30 seconds;
Steps h) and i) are conducted for a period of from 5 minutes±30 seconds;
Steps h) and i) are conducted for a period of from 6 minutes±30 seconds;
Steps h) and i) are conducted for a period of from 7 minutes±30 seconds;
Steps h) and i) are carried out concurrently;
the mechanical lysis comprises a combination of centrifugation and puck lysing;
the mechanical lysis comprises a combination of centrifugation and magnetic puck lysing;
the combination of centrifugation and puck lysing is carried out in a common lysis chamber;
Steps h) and i) are carried out concurrently;
Steps h) and i) are carried out sequentially;
Step i) is carried out after commencement of disruption of the cellular membrane in Step h);
the microorganism is susceptible to the first antibiotic agent if the quantity of the nucleic acid molecule in the control portion is more than the quantity of the nucleic acid molecule in the first test portion at the conclusion of the first incubation period;
the microorganism is susceptible to the second antibiotic agent if the quantity of the nucleic acid molecule in the control portion is more than the quantity of the nucleic acid molecule in the second test portion at the conclusion of the second incubation period;
the microorganism is not susceptible to the first antibiotic agent if the quantity of the nucleic acid molecule in the control portion is nearly equal, equal, or less than the quantity of the nucleic acid molecule in the first test portion at the conclusion of the first incubation period;
the microorganism is not susceptible to the second antibiotic agent if the quantity of the nucleic acid molecule in the control portion is nearly equal, equal, or less than the quantity of the nucleic acid molecule in the second test portion at the conclusion of the second incubation period;
the microorganism is susceptible to the first antibiotic agent when the quantity of the nucleic acid molecule in the first test portion is about 40% or less of the quantity of the nucleic acid molecule in the control portion at the conclusion of the first incubation period;
the microorganism is susceptible to the second antibiotic agent when the quantity of the nucleic acid molecule in the second test portion is about 40% or less of the quantity of the nucleic acid molecule in the control portion at the conclusion of the second incubation period;
the microorganism is resistant to the first antibiotic agent when the quantity of the nucleic acid molecule in the first test portion is about 60% or more of the quantity of the nucleic acid molecule in the control portion at the conclusion of the first incubation period; and
the microorganism is resistant to the second antibiotic agent when the quantity of the nucleic acid molecule in the second test portion is about 60% or more of the quantity of the nucleic acid molecule in the control portion at the conclusion of the second incubation period.

In another of its aspects, the present invention relates to a method for determining the susceptibility of a microorganism in a sample to an antimicrobial agent, the method comprising: (a) inoculating a test portion of the sample in a medium containing a predetermined concentration of an antimicrobial agent; (b) inoculating a control portion of the sample in a medium that does not contain the antimicrobial agent; (c) incubating the test portion and the control portion for an incubation period that is less than 420 minutes; (d) determining a quantity of a nucleic acid molecule in the test portion and a quantity of the nucleic acid molecule in the control portion at the conclusion of the incubation; and (e) determining a susceptibility of the microorganism to the antimicrobial agent by comparing the quantity of the nucleic acid molecule in the test portion to the quantity of the nucleic acid molecule in the control portion.

Preferred embodiments of this method may include any one or a combination of any two or more of any of the following features:
the incubation period is equal to or less than 420 minutes;
the incubation period is equal to or less than 390 minutes;
the incubation period is equal to or less than 360 minutes;
the incubation period is equal to or less than 330 minutes;
the incubation period is equal to or less than 300 minutes;
the incubation period is equal to or less than 270 minutes;
the incubation period is equal to or less than 240 minutes;
the incubation period is equal to or less than 210 minutes;
the incubation period is equal to or less than 150 minutes;
the incubation period is equal to or less than 120 minutes;
the incubation period is equal to or less than 90 minutes;
the incubation period is equal to or less than 60 minutes;
the incubation period is equal to or less than 30 minutes;
the microorganism comprises prokaryotic cells;
the microorganism comprises bacteria;
the bacteria comprises Gram-negative bacteria;
the bacteria comprises Gram-positive bacteria;
the bacteria comprises an unknown bacterium when steps a) to f) of claim 283 are conducted;
nucleic acid molecule comprises at least one of deoxyribonucleic acid (DNA) and ribonucleic acid (RNA);
the nucleic acid molecule comprises RNA
the nucleic acid molecule comprises ribosomal RNA
the nucleic acid molecule comprises pre-ribosomal RNA,
the nucleic acid molecule comprises mature RNA;
the nucleic acid molecule comprises at least one of 16S rRNA and 23S rRNA;
the antimicrobial agent comprises at least one antibiotic agent;
the sample comprises an unknown cellular material;
the sample comprises mammalian cellular material;
the sample comprises human cellular material;
the sample comprises a bodily fluid;
the sample comprises an inoculant derived from a bodily fluid;
the bodily fluid is selected from the group consisting of blood, urine, saliva, sweat, tears, mucus, breast milk, plasma, serum, synovial fluid, pleural fluid, lymph fluid, amniotic fluid, feces, cerebrospinal fluid, and any mixture of two or more of these;
the bodily fluid is urine or an inoculant derived therefrom;
the bodily fluid is blood or an inoculant derived therefrom;
the antibiotic agent is a bactericidal antibiotic;
the antibiotic agent is a bacteriostatic antibiotic;
the antibiotic agent comprises at least one of Gentamicin, Ciprofloxacin, Cefazolin,
Ceftriaxone, Cefepime, Ampicillin, Trimethoprim-Sulfamethoxazole, Nitrofurantoin, Fosfomycin, Amoxicillin-Clavulanate, Amikacin, Ertapenem, Meropenem and combinations thereof;
the predetermined concentration is above the sensitive CLSI MIC cutoff (for urine) for the antibiotic agent;
the predetermined concentration is above the intermediate CLSI MIC cutoff (for urine) for the antibiotic agent;
the predetermined concentration is above the resistant CLSI MIC cutoff (for urine) for the antibiotic agent;
the predetermined concentration is at least 2-fold or greater than the resistant CLSI MIC cutoff (for urine) for the antibiotic agent;
the predetermined concentration is at least 4-fold or greater than the resistant CLSI MIC cutoff (for urine) for the antibiotic agent;
the predetermined concentration is between the intermediate CLSI MIC cutoff and the resistant CLSI MIC cutoff (for urine) for the antibiotic agent;
the predetermined concentration is below the sensitive CLSI MIC cutoff (for urine) for the antibiotic agent;
the sensitive CLSI MIC cutoff (for urine) is at least 2-fold or greater than the predetermined concentration for the antibiotic agent;
the antibiotic agent comprises Gentamicin and the predetermined concentration is between about 2 μg/mL and 16 μg/mL;
the predetermined concentration is between about 2 μg/mL and 4 μg/mL;
the predetermined concentration is about 2 μg/mL;
the predetermined concentration is about 4 μg/mL;
the sensitive CLSI MIC cutoff (for urine) for Gentamicin is equal to or greater than the predetermined concentration of the antibiotic agent;
the antibiotic agent comprises Ciprofloxacin and the predetermined concentration is between about 1 μg/mL and 8 μg/mL;
The method of claim 333, wherein the predetermined concentration is between about 1 μg/mL and 4 μg/mL;
the predetermined concentration is about 4 μg/mL;
the predetermined concentration is substantially equal to the resistant CLSI MIC cutoff (for urine) for Ciprofloxacin;
the antibiotic agent comprises Cefazolin and the predetermined concentration is between about 2 μg/mL and about 256 μg/mL;
the predetermined concentration is between about 16 μg/mL and about 128 μg/mL;
the predetermined concentration is about 64 μg/mL;
the predetermined concentration is substantially equal to 2 times the resistant CLSI MIC cutoff (for urine) for Cefazolin;
the antibiotic agent comprises Ceftriaxone and the predetermined concentration is between about 1 μg/mL and about 128 μg/mL;
the predetermined concentration is between about 16 μg/mL and about 64 μg/mL;
the predetermined concentration is about 32 μg/mL;
the predetermined concentration is substantially equal to 8 times the resistant CLSI MIC cutoff (for urine) for Ceftriaxone;
the antibiotic agent comprises Cefepime and the predetermined concentration is between about 4 μg/mL and about 128 μg/mL;
the predetermined concentration is between about 16 μg/mL and about 128 μg/mL;
the predetermined concentration is between about 32 μg/mL and about 64 μg/mL;
the predetermined concentration is about 32 μg/mL;
the predetermined concentration is about 64 μg/mL;
the predetermined concentration is substantially equal to 2 or 4 times the resistant CLSI MIC cutoff (for urine) for Cefepime;
the antibiotic agent comprises Ampicillin and the predetermined concentration is between about 8 μg/mL and about 2048 μg/mL;
the predetermined concentration is between about 128 μg/mL and about 512 μg/mL;
the predetermined concentration is about 128 μg/mL;
the predetermined concentration is about 512 μg/mL;
the predetermined concentration is substantially equal to about 4 times the resistant CLSI MIC cutoff (for urine) for Ampicillin;
the predetermined concentration is substantially equal to about 16 times the resistant CLSI MIC cutoff (for urine) for Ampicillin;
the antibiotic agent comprises Trimethoprim-Sulfamethoxazole and the predetermined concentration for Trimethoprim is between about 2 μg/mL and about 16 μg/mL and the predetermined concentration for Sulfamethoxazole is between about 38 μg/mL and about 304 μg/mL;
the predetermined concentration for Trimethoprim is between about 4 μg/mL and about 8 μg/mL and the predetermined concentration for Sulfamethoxazole is between about 76 μg/mL and about 152 μg/mL;
the predetermined concentration for Trimethoprim is about 4 μg/mL and the predetermined concentration for Sulfamethoxazole is about 76 μg/mL;
the predetermined concentration for Trimethoprim-Sulfamethoxazole is substantially equal to the resistant CLSI MIC cutoff (for urine) for Trimethoprim-Sulfamethoxazole;
the antibiotic agent comprises Nitrofurantoin and the predetermined concentration is between about 4 μg/mL and about 512 μg/mL;
the predetermined concentration is between about 8 μg/mL and about 32 μg/mL;
the predetermined concentration is about 16 μg/mL;
the sensitive CLSI MIC cutoff (for urine) for Nitrofurantoin is at least 2-fold or greater than the predetermined concentration;
the antibiotic agent comprises Fosfomycin and the predetermined concentration is between about 4 μg/mL and about 512 μg/mL;
the predetermined concentration is between about 8 μg/mL and about 128 μg/mL;
the predetermined concentration is about 64 μg/mL;
the sensitive CLSI MIC cutoff (for urine) for Fosfomycin is at about equal to the predetermined concentration;
the antibiotic agent comprises Amoxicillin-Clavulanate and the predetermined concentration for Amoxicillin is between about 2 μg/mL and about 256 μg/mL and the predetermined concentration for Clavulanate is between about 1 μg/mL and about 128 μg/mL;
the predetermined concentration for Amoxicillin is between about 8 μg/mL and about 128 μg/mL and the predetermined concentration for Clavulanate is between about 4 μg/mL and about 64 μg/mL;
the predetermined concentration for Amoxicillin is about 64 μg/mL and the predetermined concentration for Clavulanate is about 32 μg/mL;
the predetermined concentration for Amoxicillin is about 32 μg/mL and the predetermined concentration for Clavulanate is about 16 μg/mL;
the predetermined concentration for Amoxicillin is about 16 μg/mL and the predetermined concentration for Clavulanate is about 8 μg/mL;
the predetermined concentration is equal to the intermediate CLSI MIC cutoff (for urine) for Amoxicillin-Clavulanate;
the predetermined concentration is greater than the intermediate CLSI MIC cutoff (for urine) for Amoxicillin-Clavulanate;
the predetermined concentration is equal to or greater than the resistant CLSI MIC cutoff (for urine) for Amoxicillin-Clavulanate;
the antibiotic agent comprises Amikacin and the predetermined concentration is between about 2 μg/mL and about 64 μg/mL;
the predetermined concentration is between about 8 μg/mL and about 64 μg/mL;
the predetermined concentration is about 32 μg/mL;
the predetermined concentration is about 16 μg/mL;
the predetermined concentration is about 8 μg/mL;
the predetermined concentration is less than the resistant CLSI MIC cutoff (for urine) for Amikacin;
the predetermined concentration is equal to the intermediate CLSI MIC cutoff (for urine) for Amikacin;
the predetermined concentration is less than or equal to the sensitive CLSI MIC cutoff (for urine) for Amikacin;
the antibiotic agent comprises Ertapenem and the predetermined concentration is between about 0;5 μg/mL and about 8 μg/mL;
the predetermined concentration is between about 1 μg/mL and about 4 μg/mL;
the predetermined concentration is about 4 μg/mL;
the predetermined concentration is about 2 μg/mL;
the predetermined concentration is greater than or equal to the resistant CLSI MIC cutoff (for urine) for Ertapenem;
the antibiotic agent comprises Meropenem and the predetermined concentration is between about 1 μg/mL and about 8 μg/mL;
the predetermined concentration is between about 1 μg/mL and about 4 μg/mL;
the predetermined concentration is about 4 μg/mL;
the predetermined concentration is about 2 μg/mL;
the predetermined concentration is equal to the resistant CLSI MIC cutoff (for urine) for Meropenem;
the predetermined concentration is equal to the intermediate CLSI MIC cutoff (for urine) for Meropenem;
determining a baseline quantity of the nucleic acid molecule in the control sample before the incubation period is complete and comparing the baseline quantity of the nucleic acid molecule to the quantity of the nucleic acid molecule in the control portion at the conclusion of the incubation period to determine if quantity of the microorganism in the control portion increased by a measurement threshold amount during the incubation period;
lysing the sample prior to determining a quantity of the nucleic acid molecule in the test portion;
further comprising the steps of
   f) subjecting the test portion to mechanical lysis to cause disruption of a cellular membrane in the microorganism;
   g) contacting the test portion with an alkaline material to produce a lysate composition comprising the nucleic acid molecule; and
   h) recovering the lysate composition from the test portion.
Step g) comprises contacting the microorganism in the test portion with an alkaline liquid;
Step g) comprises contacting the microorganism in the test portion with an alkaline solution;
the alkaline solution is a sodium hydroxide solution;
the alkaline solution has a concentration of 10 M or less;
the alkaline solution has a concentration in the range of from 1 M to 5M;
the alkaline solution has a concentration in the range of from 1;5 M to 3M;
the alkaline solution has a concentration of 2M;
the alkaline solution has a concentration of 3M;
lysing the test portion comprises transferring an aliquot of an inoculate to a lysing container;
incubating the test portion is done within a test incubation chamber on a centrifugal disc, and lysing the test portion is conducted within a lysing chamber on the same centrifugal disc;
the lysing chamber is fluidically connected to the test incubation chamber;
the lysing chamber comprises the test incubation chamber;
Steps f) and g) are conducted for a period of 10 minutes or less;
Steps f) and g) are conducted for a period of from 30 seconds to 10 minutes;
Steps f) and g) are conducted for a period of from 1 minute to 8 minutes;
Steps f) and g) are conducted for a period of from 2 minutes±30 seconds;
Steps f) and g) are conducted for a period of from 3 minutes±30 seconds;
Steps f) and g) are conducted for a period of from 4 minutes±30 seconds;
Steps f) and g) are conducted for a period of from 5 minutes±30 seconds;
Steps f) and g) are conducted for a period of from 6 minutes±30 seconds;
Steps f) and g) are conducted for a period of from 7 minutes±30 seconds;
Steps f) and g) are carried out concurrently;
the mechanical lysis comprises a combination of centrifugation and puck lysing;
the mechanical lysis comprises a combination of centrifugation and magnetic puck lysing;
the combination of centrifugation and puck lysing is carried out in a common lysis chamber;
Steps f) and g) are carried out concurrently;
Steps f) and g) are carried out sequentially;
Step g) is carried out after commencement of disruption of the cellular membrane in Step f);
the microorganism is susceptible to the antibiotic agent if the quantity of the nucleic acid molecule in the control portion is more than the quantity of the nucleic acid molecule in the test portion at the conclusion of the incubation period;
the microorganism is not susceptible to the antibiotic agent if the quantity of the nucleic acid molecule in the control portion is nearly equal, equal, or less than the quantity of the nucleic acid molecule in the test portion at the conclusion of the incubation period;
the microorganism is susceptible to the antibiotic agent when the quantity of the nucleic acid molecule in the test portion is about 40% or less of the quantity of the nucleic acid molecule in the control portion at the conclusion of the incubation period; and
the microorganism is resistant to the antibiotic agent when the quantity of the nucleic acid molecule in the test portion is about 60% or more of the quantity of the nucleic acid molecule in the control portion at the conclusion of the incubation period.

As used herein, certain terms may have the following defined meanings.

As used in the specification and claims, the singular form “a,” “an” and “the” include singular and plural references unless the context clearly dictates otherwise. For example, the term “a cell” includes a single cell as well as a plurality of cells, including mixtures thereof.

As used in the specification and claims, the term “RiboResponse™” refers to the use of a nucleic acid molecule (such as a ribosomal ribonucleic acid (“rRNA”) molecule) from a microorganism for determining the response of a cell, such as a microorganism, to an agent, such as an antimicrobial agent. That is, a molecular quantification technique utilizing nucleic acid molecules. For instance, a RiboResponse™ method for determining the susceptibility of a microorganism to an antimicrobial agent may be based on comparing the quantity of the rRNA molecules from a microorganism that has not been exposed to an antimicrobial agent to the quantity of the rRNA molecule from a microorganism that has been exposed to an antimicrobial agent.

As used in the specification and claims, as explained further below, the term “predetermined concentration” refers to an amount of an antimicrobial agent that is utilized in a test/assay to modify the test/assay to help achieve one or more objectives, such as reducing and/or minimizing an incubation period length, providing a predetermined, targeted incubation period length or reducing and/or minimizing the amount of the antimicrobial agent required to perform the test/assay in an acceptable manner.

For example, as is explained in more detail herein, at least one aspect of the teachings described herein is directed to conducting an assay using a predetermined concentration of an antimicrobial agent that has been selected to help achieve a predetermined assay objective. What the predetermined concentration amount is can differ based on the different objectives to be achieved as described herein, but is generally understood to be a concentration that is selected prior to initiating an assay to assist in performing the assay in an desired, targeted manner and to help dictate at least one aspect of the assay incubation process (such as the incubation time and/or antimicrobial usage). The predetermined concentration that is utilized in a given embodiment of the methods described herein will be based on the particular antimicrobial agent used and the particular assay-related parameter that is intended to be controlled/modified and may vary between embodiments and for different particular antimicrobial agents.

For example, some of the embodiments described herein relate to conducting an AST assay using a predetermined concentration of an antibiotic agent that has been preselected to influence at least one parameter of the incubation phase of the assay. In such embodiments, the concentration of the antibiotic agent (or other antimicrobial agent) may be selected to help alter the incubation time required to complete the assay.

Optionally, the objective of the user/operator may be to minimize the incubation time required for a given test/assay, so as to help obtain the assay results in the shortest practical time period. Alternatively, instead of minimizing the incubation time for a given assay using a given antibiotic, the objective of the user/operator may be to adjust the incubation period to meet a pre-determined, target incubation time, such as between about 90-120 minutes. In some embodiments, this may result in a targeted incubation period that meets the desired pre-determined target time limit but is actually longer than the minimum incubation time that could be achieved for that antibiotic using a different predetermined concentration. Accordingly, the predetermined concentration of an antibiotic agent that is selected by a user to provide an incubation period of about 90-120 minutes may be different than the predetermined concentration that would be selected by the user to provide the minimum incubation time for the same antibiotic agent.

Different predetermined concentrations may be utilized to target the same incubation period lengths when using different antimicrobial agents, as described herein. That is, a concentration of an antimicrobial agent that may differ from conventionally utilized concentrations for a given antimicrobial agent, and which is pre-selected to provide an incubation period, for the given antimicrobial agent, that has a desired, or target, duration (i. e., 60 minutes, 90 minutes, 120 minutes, etc.). Such concentrations can be referred to as rate-targeted concentrations. Some examples of predetermined concentrations suitable for targeting a predetermined incubation period length can include the concentrations described as “supratherapeutic” amounts as described in U.S. provisional patent application Ser. No. 62/547,361, filed Aug. 18, 2017 and Entitled Methods For Antimicrobial Susceptibility Testing, as well as the concentrations described herein.

In other embodiments, the objective of modifying the test/assay may be to reduce the amount of the antimicrobial agent used/consumed during the process while still obtaining acceptably accurate test results, without emphasis on a specific or minimized incubation period length. In such examples, the predetermined concentration appropriate to achieve the objective may differ from the predetermined concentrations that would be used if the objective was to minimize the incubation period or to target a specific incubation period length.

As explained above, when implementing the methods described herein a user may decided on a particular objective to be achieved (incubation length reduction, incubation length targeting or antimicrobial usage reduction) and based on the teachings herein may then select a predetermined concentration of a particular antimicrobial agent for use in the test/assay so as to help achieve the selected objective.

The terms “cell culture medium” and “cell culture media” used herein refer to a medium/media where a microorganism is capable of rapid growth. A cell culture medium may or may not contain at least one antimicrobial agent. In some embodiments, a cell culture medium may contain no antimicrobial agents. In some embodiments, a cell culture medium may contain one antimicrobial agent. In some embodiments, a cell culture medium may contain more than one antimicrobial agents.

The terms “specimen” or “sample” used herein refers to a material which is isolated from its natural environment, including but not limited to biological materials (see definition of “clinical specimen” below), food products, and fermented products.

The term “clinical specimen” used herein refers to samples of biological material, including but not limited to urine, blood, serum, plasma, saliva, tears, gastric and/or digestive fluids, stool, mucus, sputum, sweat, earwax, oil, semen, vaginal fluid, glandular secretion, breast milk, synovial fluid, pleural fluid, lymph fluid, amniotic fluid, feces, cerebrospinal fluid, wounds, burns, and tissue homogenates. The clinical specimen may be collected and stored by any means, including in a sterile container.

A clinical specimen may be provided by or taken from any mammal, including but not limited to humans, dogs, cats, murines, simians, farm animals, sport animals, and companion animals.

The term “incubation period” used herein refers to the period of time between when a sample is introduced into a test apparatus and allowed to grow, in the presence of a suitable media, and exposed to an antimicrobial agent (if a test sample) or not exposed to an antimicrobial agent (if a control sample) and when the growth period is stopped. The end of the incubation period may be the time at which a given sample is observed for the purpose of determining the results of the growth, or when a further action is taken with the sample that inhibits or stops the growth process. For example, in some of the examples described herein a test portion of a sample can be incubated during an incubation period that begins when a sample portion is introduced into a suitable incubation chamber and ends when the sample portion is lysed to expose some target nucleic acid molecules for counting/quantification.

The term “control portion” used herein refers to a portion of the clinical specimen which will not be exposed to an antimicrobial agent. In some embodiments, the control portion may include a plurality of portions of the clinical specimen which will not be exposed to an antimicrobial agent.

The term “test portion” used herein refers to a portion of the clinical specimen which is to be exposed to at least one antimicrobial agent. In some embodiments, the test portion may include a plurality of portions of the clinical specimen which are to be exposed to at least one antimicrobial agent. In some embodiments, the test portion may include 1, 2, 3, 4, 5, 6, 7, 8, 9, 10, 11, 12, 13, 14, 15, or more portions of the clinical specimen which are to be exposed to at least one antimicrobial agent. In some embodiments, a single test portion is exposed to one antimicrobial agent.

The term “inoculate” used herein refers to the introduction of a clinical specimen, or a portion thereof, to a culture medium. Once a clinical specimen, or a portion thereof, has been introduced into a culture medium, it may also be referred to as “an inoculate”.

The term “bacteria” used herein refers to any species of bacteria, including but not limited to Gram-negative and Gram-positive bacteria, anaerobic bacteria, and parasites. In certain embodiments, the bacteria may be Gram-negative bacteria, Gram-positive bacteria, or a mixture thereof. Examples of Gram-negative bacteria may include, but are not limited to Escherichia coli, Salmonella, Shigella, Enterobaceriaceae, Pseudomonas, Moraxella, Helicobacter, Strenotrophomonas, Bdellovibrio, and Legionella. Examples of Gram-positive bacteria may include, but are not limited to Enterococcus, Staphylococcus, Streptococcus, Actinomyces, Bacillus, Clostridium, Corynebacterium, Listeria, and Lactobacillus.

The term “bacterial density” used herein refers to the actual concentration of bacteria in a specimen. Bacterial density is expressed herein in colony forming units per milliliter (CFU/ml) but can be expressed by any other units, including but not limited to genomes per milliliter, ribosomal RNA per milliliter, or RNA molecules.

The term “bacterial density value” used herein refers to an estimate or approximation of the bacterial concentration in a specimen. The bacterial density value may refer to a species-specific concentration of bacteria or may refer to the concentration of more than one species of bacteria. Bacterial density value is expressed herein in colony forming units per milliliter (CFU/mL) but can be expressed by any other units, including but not limited to genomes per milliliter, ribosomal RNA per milliliter, or RNA molecules.

The term “rRNA” used herein refers to the ribosomal ribonucleic acid of bacteria present in a specimen.

The term “rRNA concentration” used herein refers to the number of rRNA molecules per volume tested. rRNA concentration is expressed herein in picomolar (pM) units but can be expressed by any another units.

The term “rRNA signal” used herein refers to the rRNA analyte concentration determined by the quantification of rRNA concentration in a specimen. An rRNA signal can be quantified by any known or unknown platform or method. Known platforms include but are not limited to electrochemical sensor platforms, optical platforms (e.g. ELISA, magnetic beads, capture probe arrays), and qRT-PCR.

The term “positive control” used herein refers to a known concentration of a target molecule that is included in an assay to produce a known and expected effect. Examples of target molecules that can be used as positive controls would be known to the person skilled the art, and include synthetic oligonucleotides that have the same sequence as the target rRNA sequence.

The term “negative control” used herein refers to a known treatment that is included in an assay that is not expected to have any effect. Examples of treatments that can be used as negative controls would be known to the person skilled the art, and include specimens that do not contain rRNA, including RNase-treated samples.

The term “background” used herein refers to the result obtained from samples lacking rRNA, bacteria, or other microbes.

The term “infection threshold” used herein refers to the minimum bacterial density in a clinical specimen that indicates the presence of infection. A clinical specimen with a bacterial density above the “infection threshold” therefore may suggest the presence of infection. Bacterial densities below the cutoff may be considered negative for infection, possibly indicating such factors as contamination of the specimen during collection or outgrowth of contaminants during storage or transport. The infection threshold and how it is determined may differ for the type of specimen being analyzed, for the species of bacteria being analyzed, and/or for the infection being tested for. For example, when assessing for the presence of a urinary tract infection, a false negative rate of <5% may often be sufficient for tests for bacteriuria, which may be achieved by setting the infection threshold to 2 standard deviations above background.

The term “target inoculation concentration” used herein refers to the concentration of bacteria in a clinical specimen, or a range of concentrations of bacteria in a clinical specimen, that, when inoculated into growth medium, may provide accurate results on an AST. For example, for a direct from specimen phenotypic AST of a urine specimen, an inoculation concentration of ideally 5×10⁵ CFU/mL and of no greater than 5×10⁶ CFU/mL may provide an accurate AST result, whereas inoculation concentrations more than 5×10⁶ CFU/mL may reduce the accuracy. The target inoculation concentration may be used to determine what dilution factor, if any, is required to dilute a clinical specimen such that the bacterial density of the specimen may be optimized for an AST.

As explained herein, a predetermined concentration may be different for different antimicrobial agents. For some antimicrobial agents, a “predetermined concentration” may be above the minimum concentration of the antimicrobial agent that would be used therapeutically to treat a subject with an infection of a microorganism susceptible to the antimicrobial agent. For some antimicrobial agents, a “predetermined concentration” may be equal to the minimum concentration of the antimicrobial agent that would be used therapeutically to treat a subject with an infection of a microorganism susceptible to the antimicrobial agent. For some antimicrobial agents, a “predetermined concentration” may be below the minimum concentration of the antimicrobial agent that would be used therapeutically to treat a subject with an infection of a microorganism susceptible to the antimicrobial agent.

The predetermined concentration may, in some instances, be defined in relation to the Clinical and Laboratory Standards Institute (CLSI) minimum inhibitory concentration (MIC) breakpoint for that antimicrobial agent. In some embodiments, a “predetermined concentration” of an antimicrobial agent is an amount (i.e., concentration) below the susceptible CLSI MIC breakpoint for the antimicrobial agent. In some embodiments, a “predetermined concentration” of an antimicrobial agent is an amount (i.e., concentration) above the susceptible CLSI MIC breakpoint for the antimicrobial agent. In some embodiments, a “predetermined concentration” of an antimicrobial agent is an amount between the susceptible CLSI MIC breakpoint and the intermediate CLSI MIC breakpoint for the antimicrobial agent. In some embodiments, a “predetermined concentration” of an antimicrobial agent is an amount above the intermediate CLSI MIC breakpoint for the antimicrobial agent. In some embodiments, a “predetermined concentration” of an antimicrobial agent is an amount between the intermediate CLSI MIC breakpoint and the resistant CLSI MIC breakpoint for the antimicrobial agent. In some embodiments, a “predetermined concentration” of an antimicrobial agent is an amount above the resistant CLSI MIC breakpoint for the antimicrobial agent.

The predetermined concentration(s) for a given antimicrobial agent may be determined by empirical testing, and may be collected in a database, look-up table or the like which can then be used to determine a particular predetermined concentration that should be used when trying to achieve a particular objective, such as when manipulating the performance of different antibiotic agents in a given testing circumstance/environment so that the incubation period for each antibiotic agent approaches the same target incubation period length. In the present case, the inventors have conducted tests on a variety of different antimicrobial agents and have identified a variety of potentially useful, predetermined concentrations. For example, Table 1 below shows some examples of some predetermined concentrations that can be used to achieve targeted incubation period lengths when the object of the test/assay is to provide an incubation period of about 90 minutes for the antimicrobials listed:

TABLE 1
Predetermined concentration and MIC Criteria for select antibiotics
to provide an incubation period of about 90 mins.

| | MIC Criteria (μg/mL) | | | |
|---|---|---|---|---|
| | CLSI M100 (2018) | | | Predetermined |
| | S | I | R | concentration(μg/ml) |
| Ampicillin | ≤8 | 16 | ≥32 | 128 |
| Cefazolin | ≤2 | 4 | ≥8 | 64 |
| Ceftriaxone | ≤1 | 2 | ≥4 | 32 |
| Cefepime | ≤8 | 16 | ≥32 | 64 |

Table 2 below shows additional examples of some predetermined concentrations that can be used to define incubation periods for the antimicrobials listed, as determined by additional tests conducted by the inventors in the present case:

TABLE 2
Predetermined concentration and MIC Criteria for select antibiotics to provide an
incubation period of about 90 mins.

| | CLSI MIC Cutoffs for (Urine) AST | | | | Predetermined | |
|---|---|---|---|---|---|---|
| | Determination and RiboResponse | | | | concentration(μg/mL) | |
| | Antibiotic Panels | | | | Working | |
| Antibiotic | S | SDD | I | R | Panel 1 | Working Panel 2 |
| Amikacin | ≤16 | | 32 | ≥64 | 64 | 32, 16, 8 |
| Amox/Clav | ≤8/4 | | 16/8 | ≥32/16 | 32/16 | 64/32, 32/16, 16/8 |
| Ampicillin | <8 | | 16 | ≥32 | 512 | 128 |
| Cefazolin | ≤16 | | | ≥32 | 64 | 64 |
| Cefepime | ≤2 | 4-8 | | ≥16 | 64 | 64, 32 |
| Ceftriaxone | ≤1 | | 2 | ≥4 | 32 | 32 |
| Ciprofloxacin | ≤1 | | 2 | ≥4 | 4 | 4 |
| Ertapenem | ≤0.5 | | 1 | ≥2 | | 4, 2 |
| Fosfomycin | ≤64 | | 128 | ≥256 | 64 | 64 |
| Gentamicin | ≤4 | | 8 | ≥16 | 4 | 4, 2 |
| Imipenem | ≤1 | | 2 | ≥4 | 4 | |
| Meropenem | ≤1 | | 2 | ≥4 | | 4, 2 |
| Nitrofurantoin | ≤32 | | 64 | ≥128 | 16 | 16 |
| Pip/Tazo | ≤16/4 | | 32/4-64/4 | ≥128/4 | 16/4 | |
| TMP/SMX | ≤2/38 | | | ≥4/76 | 8/152 | 4/76 |

Amox/Clav = Amoxicillin/Clavulanate, Pip/Tazo = Piperacillin/Tazobactam, TMP/SMX = Trimethoprim/Sulfamethoxazole. CLSI AST Results: S = Susceptible, I = Intermediate, R = Resistant, SDD = Susceptible dose-dependent (treatment with this drug for this MIC range require higher than normal drug exposure).

The urine cutoffs for cefazolin (S<=16, R>=32) in Table 2 are for “uncomplicated” UTI cases as identified in the CLSI. The cutoffs for cefazolin from Table 1 are suitable for use with cefazolin cases not identified as “uncomplicated”. Both cutoff ranges are listed in the CLSI M100 from 2018.

Accordingly, if a user was attempting to provide a commercially usable AST assay that could be performed with an incubation period of about 90 minutes, and using one or more of the antibiotics listed above, the commercial test apparatus could be pre-loaded with the corresponding predetermined concentrations of the antibiotics described herein. If an AST is to be developed using antibiotics or targeting a particular microorganism that was not tested or described herein, a person skilled in the art could, based on the teachings here, replicate the experimentation described and derive concentrations for the other antibiotics or microorganisms that can provide an incubation period of about 90 minutes.

In these experiments, the first working panel of antibiotics was developed to help produce acceptably accurate AST answers/results with an incubation period of between about 60-90 minutes for E. coli. While it was found that this first panel was effective on other Gram-negative bacteria and time points, it was observed that using this panel with microorganisms that differed from E. coli with an incubation period of about 60-90 tended to reduce the accuracy of the AST results.

In subsequent experiments, a second working panel of agents was then developed to help maintain the same level of AST determination accuracy across a relatively wider variety of Gram negative pathogens and targeting about 90-120 minutes incubation for both lab grown bacteria and those from clinical urine specimens. It was discovered that the increasing the incubation time was helped to extend the use of the panel to some types of relatively slower-growing bacteria and helped to facilitate adequate growth in the antibiotic free control portions. In this example, ertapenem and meropenem replaced imipenem to help provide a relatively wider coverage/detection of carbapenem resistant Enterobacteriaceae. This may also help bypass the long-term instability of imipenem in solution. Piperacillin/Tazobactam was dropped from the second working panel due to its relatively poor performance (accuracy was too low) when used in relation to non-E. coli bacteria at 90 and 120-minute incubation periods, but could remain useful using longer incubation periods and/or in assays targeting E. coli.

During the development of present assay and the specific antibiotic concentrations listed in Table 2, the initial concentration considered for each given antibiotic was inclusive of the CLSI cutoffs. It was unexpectedly discovered that the preferred rate-targeting concentrations (the working concentrations in the table) for at least some antibiotics would differ, sometimes substantially, from the CLSI cutoffs. Even more unexpected was that some of the preferred rate-targeting concentrations were below the respective susceptible cutoff and some were above their respective resistant cutoff. While the exact reason for these differences may not be fully understood, it may be that it relates to the specific mechanism of action of an antibiotic. For example, if the antibiotic acts relatively slowly on the bacterial cell, it may require a relatively higher concentration to see an appropriate result from susceptible bacteria in the relatively rapid assay window or short incubation periods. This may help reduce false resistance results. Conversely, if an antibiotic acts relatively quickly on the bacterial cell, it may require a relatively lower concentration it to obtain an accurate result, and to help reduce the chances of an inappropriate result.

It was also discovered that the rate-targeting dosages for almost all of the beta-lactam antibiotics tested in assays having incubation times of between 60-120 minutes were concentrations above the respective CLSI resistant cutoffs. The fact that these antibiotics attack the bacteria by inhibiting cell wall synthesis may be one factor that contributes to the apparent advantage of providing relatively higher antibiotic concentrations to help achieve the desired incubation times. It was also discovered that the folate synthesis inhibitor combination of trimethoprim and sulfamethoxazole, which is bacteriostatic, also appears to have a relatively higher rate-targeting concentration.

In contrast, some antibiotics, like Nitrofurantoin for example, were discovered to have rate-targeting concentrations at or below the susceptible CLSI cutoff when configured to achieve the desired 60-120-minute incubation period. These concentrations were determined to provide relatively accurate results in our selected incubation periods. It is noted that the traditional optical and microscopic techniques used to generate and validate these CLSI cutoffs would involve incubating with concentrations that are too high for the targeted incubation period(s) for certain antibiotics, which could cause resistant bacteria to incorrectly appear susceptible. This may be because the traditional tests incubate for a long enough time (several hours) with the antibiotic to help overcome any initial growth inhibition for the resistant bacteria and therefore generate reliable results. The opposite would generally apply for antibiotics that have a working concentration at or above the resistant CLSI cutoff. The traditional optical and microscopic techniques used to generate and validate these CLSI cutoffs would involves incubating with concentrations that are too low for the present methods and desired incubation period for certain antibiotics, and would cause susceptible bacteria to incorrectly appear resistant. The traditional tests incubate for enough time (several hours) with the antibiotic to overcome any initial growth for the susceptible bacteria and generate reliable results. The unique combination of the ribosomal RNA assay and unconventional, and in some cases optimized, antibiotic concentrations help facilitate the providing an incubation period of desired length while maintaining an acceptable accuracy of the AST test results.

In addition to the working panels summarized in Table 2, further experiments were conducted to help identify some upper and lower bounds on the rate-targeting concentration that can still help provide useful results, but may not be the rates that are most suitable for conducting the assay within the 60-120-minute incubation period window. Table 3 summaries the results of this testing:

TABLE 3
List of Highest and Lowest Concentrations Tested that provided
acceptable AST assay results and the related concentration
to provide a 90-120-minute incubation period.

| | Upper | Lower | |
|---|---|---|---|
| | Concentration | Concentration | 90-120 Minute |
| Antibiotic | Limit | Limit | Incubation |
| Amikacin | 64 | 2 | 32, 16, 8 |
| Amoxicillin/ | 256/128 | 2/1 | 64/32, 32/16, 16/8 |
| Clavulanate | | | |
| Ampicillin | 2048 | 8 | 128 |
| Cefazolin | 256 | 2 | 64 |
| Cefepime | 128 | 4 | 64, 32 |
| Ceftriaxone | 128 | 1 | 32 |
| Ciprofloxacin | 8 | 1 | 4 |
| Ertapenem | 8 | 0.5 | 4, 2 |
| Fosfomycin | 512 | 4 | 64 |
| Gentamicin | 16 | 2 | 4, 2 |
| Meropenem | 8 | 1 | 4, 2 |
| Nitrofurantoin | 512 | 4 | 16 |
| TMP/SMX | 16/304 | 2/38 | 4/76 |

Concentrations are listed in μg/ml;
TMP/SMX = Trimethoprim/Sulfamethoxazole

If the objective of a given user is to minimize the incubation time when using one of the listed antibiotics, the values from column 1 may be the suitable predetermined concentration. If the objective is to provide an incubation period of 90-120 minutes, the values from column 3 may be the suitable predetermined concentration. If the objective is to consume the minimal amount of the antibiotic agent while still obtaining accurate results, the values from column 2 may be the suitable predetermined concentration.

The methods disclosed herein comprise the use of one or more different antimicrobial agents. Use of one or more antimicrobial agents may comprise producing an inoculate comprising a microorganism in a cell culture media containing one or more antimicrobial agents. Use of one or more antimicrobial agents may comprise obtaining an inoculate comprising a microorganism in a cell culture media containing one or more antimicrobial agents. Use of one or more antimicrobial agents may comprise exposing a microorganism to one or more antimicrobial agents.

Testing was conducted on a variety of different antimicrobial agents to help identify one or more potentially useful concentrations for the different agents. Based on this testing, which included the experiments described herein, a variety of different concentrations for different antimicrobial agents, and for different testing objectives, were discovered.

Based on these predetermined concentrations, one or more methods for determining the susceptibility of a microorganism in a sample to a given antimicrobial agent can include the steps of dividing the sample into at least one test portion and at least one control portion and incubating the test portion in the presence of the predetermined concentration and separately incubating the control portion. At the end of the incubation period, both portions can be further processed if desired (such as via lysing) and the relative amounts of a target nucleic acid molecule (such as DNA or RNA) in each of the portions can be determined. The microorganism can be considered to be susceptible to the particular antimicrobial agent if the concentration of the target nucleic acid molecule in the test portion is below a susceptibility cutoff level or threshold, can be considered to be resistant if the concentration of the target nucleic acid molecule in the test portion is above a resistant cutoff level or threshold, and may be considered indeterminate if the concentration of the target nucleic acid molecule in the test portion is between the susceptibility and resistant thresholds.

For example, in some of the experiments discussed herein, the susceptibility threshold was selected to be about 40% of the concentration of the target nucleic acid molecule in the control portion and the resistant threshold was selected to be about 60% of the concentration of the target nucleic acid molecule in the control portion. That is, for example, a microorganism was considered to be susceptible to a given antimicrobial agent if the concentration of rRNA in the test portion was less than or equal to about 40% of the concentration of rRNA in the antibiotic free control portion, resistant if the concentration of rRNA in the test portion was greater than or equal to about 60% of the concentration of rRNA in the antibiotic free control, and the results were considered to be indeterminate if the concentration of rRNA in the test portion was between about 40% and about 60% of the concentration of rRNA in the antibiotic free control.

It was discovered that at least some of these threshold values may be further refined for a given antibiotic agent and when being specifically used in combination with an incubation period of between about 90-120 minutes, while maintaining and/or enhancing accuracy (when compared to the standard). This was found to be effective across a variety of gram negative pathogens. Table 4 below summarize the experimental findings related to the threshold values for certain, tested antibiotic agents. Such thresholds could be incorporated into an automated instrument's software program to help facilitate the automated interpretation of the results by comparison to reference criteria.

TABLE 4
Antibiotic Specific-Cutoffs for RiboResponse AST Interpretation (% of Antibiotic Free) for
(a) E. coli and (b) K. pneumoniae.
(a) E. coli

| | Amox/Clav | Amp | Cefaz | Ceftriax | Cipro | Gent | Nitro | TMP/SMX |
|---|---|---|---|---|---|---|---|---|
| Susceptible | ≤65 | ≤55 | ≤45 | ≤65 | ≤40 | ≤65 | ≤55 | ≤69 |
| Indeterminate | 66-85 | 56-70 | 46-70 | 66-80 | 41-70 | 66-80 | 56-60 | 70-75 |
| Resistant | ≥86 | ≥71 | ≥71 | ≥81 | ≥71 | ≥81 | ≥61 | ≥76 |

(b) K. pneumoniae

| | Amox/Clav | Cefaz | Cetriax | Cipro | Nitro | TMP/SMX |
|---|---|---|---|---|---|---|
| Susceptible | ≤65 | ≤55 | ≤65 | ≤35 | ≤25 | ≤65 |
| Indeterminate | 66-85 | 56-65 | 66-75 | 36-60 | 26-60 | 66-75 |
| Resistant | ≥86 | ≥66 | ≥76 | ≥61 | ≥61 | ≥76 |

To test the accuracy of the methods described herein, the assay was conducted in parallel to the traditional (slow) method on blinded urine specimens. Accuracy for this study was measured as how well the RiboResponse AST answer for a given specimen and antibiotic compared to that of broth microdilution (through UCLA Clinical Microbiology Laboratory). By combining the ribosomal RNA assay with incredibly well-optimized antibiotic concentrations, the rapid AST assay, utilizing RiboResponse, was able to generate results within hours of specimen collection, that normally take days, with 96% accuracy. It is believed that a majority of the errors were caused by specimens containing a mixture of susceptibility phenotypes, rather than a failure of the assay techniques themselves. In these cases, the results measured the mixture of the phenotypes present in the specimen and thus may still be used to inform treatment of the mixed infection in some cases.

As described herein, these threshold values were selected to yield acceptable accuracy (when compared to the results obtained using broth microdilution) with the concentrations used to conduct the AST analysis with incubation periods of only 90-120 minutes across a variety of different microorganisms (including different gram-negative pathogens). By way of example, the inventors have assessed the accuracy of the present invention (conducted with an incubation period of 90-120 minutes) to determine susceptibility of microorganisms in a urine specimen to a variety of antimicrobials, as compared to an AST conducted on the same samples using broth microdilution. These example results are set out in Table 5:

TABLE 5
Accuracy of Present Invention (i.e., RiboResponse-Based AST)
for Urine Specimen Compared to the Conventional Testing Method

| | | | | Very | | Accuracy |
|---|---|---|---|---|---|---|
| | | Minor | Major | Major | | of Present |
| Antibiotic | Correct | Error¹ | Error² | Error³ | Mixed⁴ | Invention |
| Amikacin | 27 | 0 | 0 | 0 | 0 | 100.0% |
| Amoxicillin/ | 23 | 1 | 0 | 0 | 0 | 95.8% |
| Clavulanate | | | | | | |
| Ampicillin | 39 | 0 | 0 | 0 | 2 | 95.1% |
| Cefazolin | 41 | 0 | 0 | 0 | 1 | 97.6% |
| Ceftriaxone | 40 | 0 | 0 | 0 | 1 | 97.6% |
| Ciprofloxacin | 41 | 1 | 0 | 0 | 0 | 97.6% |
| Gentamicin | 42 | 0 | 0 | 0 | 0 | 100.0% |
| Nitrofurantoin | 28 | 1 | 0 | 0 | 1 | 93.3% |
| Trimethoprim/ | 38 | 0 | 1 | 0 | 3 | 90.5% |
| Sulfamethoxazole | | | | | | |
| Total | 319 | 3 | 1 | 0 | 8 | 96.4% |

¹Minor error = Intermediate AST result from gold standard,
²Major error = False resistance from RiboResponse,
³Very major error = false susceptibility from RiboResponse,
⁴Mixed = specimens containing a mixture of antibiotic susceptibility phenotypes (e.g., one specimen containing two different E. coli strains, one susceptible to Trimethoprim/Sulfamethoxazole, one resistant).

Other ranges or thresholds may be used for different examples or adaptations of the methods described herein.

### Inoculation

In some embodiments, the medium into which the test and/or control portions of the clinical specimen are inoculated is in a container. In some embodiments, the container is selected from the group of a tissue culture plate, vial, flask, microcentrifuge tube, and centrifugal disk. In some embodiments, the container is a well of a tissue culture plate. In some embodiments, the tissue culture plate contains a plurality of wells (i.e., any number of wells). In some embodiments, the tissue culture plate contains 6, 12, 24, 48, 96, or more wells. In some embodiments, the container is a chamber of a centrifugal disc.

Optionally, multiple test chambers can be included in a single centrifugal disc, such that more than one test can be conducted using a common apparatus, but preferably in fluid isolation from each other.

Optionally, more than one process step/phase can be conducted within a common container or chamber within the container. For example, the steps of inoculation, incubation and lysing may be performed in a single chamber. This may help reduce the size and/or complexity of the testing apparatus and/or centrifugal disc. In such arrangements, the lysing agents/mechanisms, may be inactive during the incubation period or otherwise configured so as not to interrupt the incubation of a given test portion until a desired processing time. For example, mechanical lysing agents may be held in a static position and/or chemical lysing agents may be encapsulated, segregated from the test portion during incubation, introduced into the chamber at the conclusion of the incubation period or otherwise manipulated to only take effect at a desired time.

### Incubation

In some embodiments, the one or more inoculates are shaken. In some embodiments, shaking an inoculate comprises placing the container with an inoculate in a shaking incubator. In some embodiments, shaking an inoculate comprises shaking the container with the inoculate at 400 or more revolutions per minute (rpm). In some embodiments, shaking the inoculate occurs prior to determining the quantity of a nucleic acid molecule in a plurality of inoculates.

In some embodiments, turbulence is generated by alternately accelerating and decelerating the inoculate. In some embodiments, generating turbulence comprises placing the container with the inoculate on a rotating platform where the rotation alternately accelerates and decelerates. In some embodiments, generating turbulence occurs prior to determining the quantity of a nucleic acid molecule in a plurality of inoculates. Methods for using a rotating platform for improving growth of a microorganism in a liquid culture have been disclosed in provisional patent application S.N. 62/552,332, filed Aug. 30, 2017, the contents of which are hereby incorporated by reference herein in its entirety.

In some embodiments, the inoculates are incubated at 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., or 42° C. In some embodiments, the inoculates are incubated at 25° C. In some embodiments, the inoculates are incubated at 30° C. Preferably, the inoculates are incubated at about 37° C.

In some embodiments, the inoculates are incubated at suitable temperatures for at least 30, 60, 90, 120, 150, 180, 210, 240, 270, 300, 360, or 420 or more minutes. In some embodiments, the inoculates are incubated for at least 60 minutes. In some embodiments, the inoculates are incubated for at least 90 minutes. In some embodiments, the inoculates are incubated for at least 120 minutes. In some embodiments, the inoculates are incubated for less than 420 minutes, less than 360 minutes, less than 300 minutes, less than 270 minutes, less than 240 minutes, less than 210 minutes, less than 180 minutes, less than 150 minutes, less than 120 minutes, less than 90 minutes, less than 60 minutes, or less than 30 minutes.

In some embodiments, the inoculates are incubated at 37° C. for at least 30, 60, 90, 120, 150, 180, 210, 240, 270, 300, 360 or 420 or more minutes. In some embodiments, the inoculates are incubated at 37° C. for at least 60 minutes. In some embodiments, the inoculates are incubated at 37° C. for at least 90 minutes. In some embodiments, the inoculates are incubated at 37° C. for at least 120 minutes. In some embodiments, the inoculates are incubated for less than 420 minutes, less than 360 minutes, less than 300 minutes, less than 270 minutes, less than 240 minutes, less than 210 minutes, less than 180 minutes, less than 150 minutes, less than 120 minutes, less than 90 minutes, less than 60 minutes, or less than 30 minutes. Preferably, the inoculates are incubated for less than 120 minutes,

### Lysis

Methods for lysing the microorganism in an inoculate to produce a cell lysate have been disclosed in PCT/US18/45211, which is incorporated herein by reference herein in its entirety.

In some embodiments, the methods disclosed herein comprise steps for extracting a target chemical compound from a cellular material in a sample, the steps comprising (a) subjecting the sample to mechanical lysis to cause disruption of a cellular membrane in the cellular material; (b) contacting the sample with an alkaline material to produce a lysate composition comprising the target chemical compound; and (c) recovering the lysate composition from the sample, wherein the target chemical sample may be a nucleic acid. In some embodiments, the nucleic acid may be deoxyribonucleic acid (DNA). Examples of RNA involved in protein synthesis include, but are not limited to, messenger RNA (mRNA), transfer RNA (tRNA), transfer-messenger RNA (tmRNA), single recognition particle RNA (SRP RNA), and ribosomal RNA (rRNA). In some embodiments, the nucleic acid may be ribonucleic acid (RNA). In certain preferred embodiments, the nucleic acid may be ribosomal RNA (rRNA), or more preferably may pre-ribosomal rRNA, mature rRNA, or may be selected from the group consisting of 16S rRNA, 23S rRNA or any mixture thereof.

Provided in another embodiment is steps for extracting a target chemical compound from a cellular material in a sample, the steps comprising (a) subjecting the sample to mechanical lysis to cause disruption of a cellular membrane in the cellular material; (b) contacting the sample with an alkaline material to produce a lysate composition comprising the target chemical compound; and (c) recovering the lysate composition from the sample, wherein step (b) may comprise contacting the cellular material in the sample with an alkaline solution. In some embodiments, the alkaline solution may be a sodium hydroxide solution. In certain preferred embodiments, the alkaline solution may have a concentration of about 10 M or less, preferably of about 1 M to 5M, and more preferably of about 1.5 M to 3M. In certain preferred embodiments, the alkaline solution may have a concentration of about 2M. In other preferred embodiments, the alkaline solution may have a concentration of about 3M.

Provided in another embodiment is steps for extracting a target chemical compound from a cellular material in a sample, the method comprising (a) subjecting the sample to mechanical lysis to cause disruption of a cellular membrane in the cellular material; (b) contacting the sample with an alkaline material to produce a lysate composition comprising the target chemical compound; and (c) recovering the lysate composition from the sample, wherein the cellular material may be an unknown cellular material.

Provided in another embodiment is steps for extracting a target chemical compound from a cellular material in a sample, the steps comprising (a) subjecting the sample to mechanical lysis to cause disruption of a cellular membrane in the cellular material; (b) contacting the sample with an alkaline material to produce a lysate composition comprising the target chemical compound; and (c) recovering the lysate composition from the sample, wherein the cellular material may be either a microorganism, prokaryotic cells, virally infected cells, fungus cells, or yeast cells. Examples of yeast cells may include but are not limited to Candida cells. Methods for detecting the presence of a fungal organisms within a biological sample, such as yeast have been disclosed in International Patent Publication No. WO 2013166460 and WO 2015013324, both of which are incorporated herein by reference herein in their entirety.

Provided in another embodiment is steps for extracting a target chemical compound from a cellular material in a sample, the steps comprising (a) subjecting the sample to mechanical lysis to cause disruption of a cellular membrane in the cellular material; (b) contacting the sample with an alkaline material to produce a lysate composition comprising the target chemical compound; and (c) recovering the lysate composition from the sample, wherein the cellular material may be bacteria.

Provided in another embodiment is steps for extracting a target chemical compound from a cellular material in a sample, the steps comprising (a) subjecting the sample to mechanical lysis to cause disruption of a cellular membrane in the cellular material; (b) contacting the sample with an alkaline material to produce a lysate composition comprising the target chemical compound; and (c) recovering the lysate composition from the sample, wherein the sample may comprise mammalian cellular material, preferably human cellular material, and more preferably a bodily fluid or an inoculant derived therefrom. In certain preferred embodiments, the bodily fluid may be selected from the group consisting of blood, urine, saliva, sweat, tears, mucus, breast milk, plasma, serum, synovial fluid, pleural fluid, lymph fluid, amniotic fluid, feces, cerebrospinal fluid and any mixture of two or more of these. Other examples of mammalian cellular material include but are not limited to samples from monkeys, cats, dogs, sheep, goats, cows, pigs, horses, or rabbits.

Provided in another embodiment is a method for extracting a target chemical compound from a cellular material in a sample, the method comprising (a) subjecting the sample to mechanical lysis to cause disruption of a cellular membrane in the cellular material; (b) contacting the sample with an alkaline material to produce a lysate composition comprising the target chemical compound; and (c) recovering the lysate composition from the sample, wherein after disruption of the cellular membrane in the cellular material, the sample may be subjected to biological lysis. In some embodiments, the biological lysis may include contacting the sample with an enzyme. In certain preferred embodiments, the enzyme may be selected from the group consisting of lysozyme, lysostaphin and any mixture thereof.

Provided in another embodiment is a method for extracting a target chemical compound from a cellular material in a sample, the method comprising (a) subjecting the sample to mechanical lysis to cause disruption of a cellular membrane in the cellular material; (b) contacting the sample with an alkaline material to produce a lysate composition comprising the target chemical compound; and (c) recovering the lysate composition from the sample, wherein after disruption of the cellular membrane in the cellular material, the sample may be subjected to physical lysis. In some embodiments, the physical lysis may be selected from the group consisting of heating, osmotic shock, cavitation or any combination of two or more of these. Physical lysis methods such as those mentioned above are common in the art. For example, lysis by heating may comprise placing the sample in a water bath, heat block, or temperature controlled container, where the temperature of the water bath, heat block, or temperature controlled container may be less than or equal to about 100° C., preferably between about 40° C. and about 100° C., or more preferably the sample may be heated at 45° C., 50° C., 55° C., 60° C., 65° C., 70° C., 75° C., 80° C., 85° C., 90° C., or 95° C. Cavitation may comprise nitrogen cavitation which may be performed by (a) placing cells from a sample in a pressure vessel; (b) dissolving oxygen-free nitrogen in the cells under high pressure; and (c) releasing the pressure in the vessel. Osmotic shock may be performed by changing the concentration of a salt, substrate or solute around cells from a sample, such that the cells rupture and/or release intracellular materials, such as nucleic acid molecules and proteins.

Provided in another embodiment is a method for extracting a target chemical compound from a cellular material in a sample, the method comprising (a) subjecting the sample to mechanical lysis to cause disruption of a cellular membrane in the cellular material; (b) contacting the sample with an alkaline material to produce a lysate composition comprising the target chemical compound; and (c) recovering the lysate composition from the sample, wherein step (a) may be conducted for a period of about 10 minutes or less, preferably from about 30 seconds to about 10 minutes, more preferably from about 1 minute to 8 minutes, and most preferably for a period of about 2 minutes±30 seconds, about 3 minutes±30 seconds, about 4 minutes±30 seconds, about 5 minutes±30 seconds, about 6 minutes±30 seconds, or about 7 minutes±30 seconds.

Provided in another embodiment is a method for extracting a target chemical compound from a cellular material in a sample, the method comprising (a) subjecting the sample to mechanical lysis to cause disruption of a cellular membrane in the cellular material; (b) contacting the sample with an alkaline material to produce a lysate composition comprising the target chemical compound; and (c) recovering the lysate composition from the sample, wherein the mechanical lysis may be selected from the group consisting of French press, shaking, grinding, bead beating, centrifugation and any combination of two or more of these. For example, lysis by French press may performed by passing a sample through a narrow valve under high pressure. Lysis by grinding may be performed by placing a sample in a grinder. Examples of grinders may include, but are not limited to, a ball mill, coffee grinder, Geno/Grinder, and Retsch Mixer Mill. A ball mill for instance, may comprise a hollow cylindrical shell and one or more balls, where the balls may be made of chrome steel, stainless steel, ceramic, or rubber. Lysis by grinding may comprise, for example, the use of a mortar and pestle. Lysis by shaking may comprise, for example, mixing the sample with some sort of bead or matrix, and placing the sample on a violent high-speed shaker.

In some embodiments, where the mechanical lysis is performed by bead beating, said bead beating my comprise beating the sample with ceramic beads, glass beads, zirconium beads, silica-zirconium beads, steel beads or any combination of two or more of these. In certain preferred embodiments, bead beating may comprise the use of magnetic beads. By way of non-limiting example, silica-zirconium beads may be preferable for use in the disclose inventions as they are chemically inert and have been shown not to interfere with the assay techniques.

Provided in another embodiment is a method for extracting a target chemical compound from a cellular material in a sample, the method comprising (a) subjecting the sample to mechanical lysis to cause disruption of a cellular membrane in the cellular material; (b) contacting the sample with an alkaline material to produce a lysate composition comprising the target chemical compound; and (c) recovering the lysate composition from the sample, wherein the mechanical lysis may comprise using OmniLyse® or a functional equivalent thereof. Mechanic lysis with OmniLyse® or a functional equivalent thereof, for instance, may comprise the use of a small chamber containing, for example, zirconium beads, where the chamber is then connected to a syringe and a motor. By way of non-limiting example, OmniLyse® lysis may comprise drawing a solution into the chamber with the syringe and turning on the motor to move the beads around at around 30,000 rpm with a small propeller, then ejecting the solution back into a tube using the syringe.

Provided in another embodiment is a method for extracting a target chemical compound from a cellular material in a sample, the method comprising (a) subjecting the sample to mechanical lysis to cause disruption of a cellular membrane in the cellular material; (b) contacting the sample with an alkaline material to produce a lysate composition comprising the target chemical compound; and (c) recovering the lysate composition from the sample, wherein the mechanical lysis may comprise a combination of centrifugation and puck lysing. In some embodiments, the puck lysing may be magnetic puck lysing. In certain preferred embodiments, the combination of centrifugation and disk lysing may be carried out in a common lysis chamber, where preferably centrifugation and puck lysing may be carried out on a centrifugal disk (CD). By way of non-limiting example, the centrifugal disk may comprise one or more microfluidic lysis chambers connected to one another by one or more microfluidic channels, where at least one of the microfluidic lysis chambers has an inlet port which may be configured to receive a fluid sample. Each lysis chamber of the CD may contain one or more magnetic lysis pucks and a series of beads, wherein the lysis pucks and beads are small enough to be able to move within the lysis chamber, but not small enough to exit the lysis chamber through any of the microfluidic channels. The CD may be configured to fit on a rotating platform connected to a motor, such that when the CD is placed on the platform and the motor is turned on, the CD will rotate. The platform my further comprise a series of stationary magnets which may be configured such that when the CD is rotating, the interaction between the stationary magnets and the magnetic lysis pucks causes the lysis pucks to move back and forth within each of the one or more lysis chambers. Lysis methods such as this are known in the art, including those disclosed in U.S. Pat. No. 8,303,911 which is incorporated by reference herein in its entirety.

Provided in another embodiment is a method for extracting a target chemical compound from a cellular material in a sample, the method comprising (a) subjecting the sample to mechanical lysis to cause disruption of a cellular membrane in the cellular material; (b) contacting the sample with an alkaline material to produce a lysate composition comprising the target chemical compound; and (c) recovering the lysate composition from the sample, wherein steps (a) and (b) may be carried out concurrently.

Provided in another embodiment is a method for extracting a target chemical compound from a cellular material in a sample, the method comprising (a) subjecting the sample to mechanical lysis to cause disruption of a cellular membrane in the cellular material; (b) contacting the sample with an alkaline material to produce a lysate composition comprising the target chemical compound; and (c) recovering the lysate composition from the sample, wherein steps (a) and (b) may be carried out sequentially. In certain preferred embodiments, step (b) may be carried out after commencement of disruption of the cellular membrane in step (a). This sequential method may be preferred because alkaline lysing alone will not be able to disrupt the cellular membrane of Gram-positive cells and/or fungal cells. Thus, in order to get access to the target compound within a Gram-positive and/or fungal cell, the cellular membrane is disrupted by the shear forces of mechanical lysing.

Provided in another embodiment is a method for extracting a target chemical compound from a cellular material in a sample, the method comprising (a) subjecting the sample to mechanical lysis to cause disruption of a cellular membrane in the cellular material; (b) contacting the sample with an alkaline material to produce a lysate composition comprising the target chemical compound; and (c) recovering the lysate composition from the sample, wherein the method further comprises neutralizing the sample by contacting the sample with a buffer solution. When a sample is contacted with an alkaline solution, high concentrations of hydroxide ions break apart the protein components of a cell ribosome, unwind the secondary structure of rRNA, and break it into pieces. If this process is left unchecked, it will eventually break down the entire rRNA into single bases. In order to arrest this process, a concentrated buffer solution may be added to neutralize the pH of the lysate. In some embodiments, the buffer solution may be a phosphate buffer solution. In certain preferred embodiments the buffer solution may have a pH of less than 7, preferably in the range of about 5 to 7.5, and more preferably in the range of 6 to 7.

Provided in another embodiment is a method for extracting a target chemical compound from a cellular material in a sample, the method comprising (a) subjecting the sample to mechanical lysis to cause disruption of a cellular membrane in the cellular material; (b) contacting the sample with an alkaline material to produce a lysate composition comprising the target chemical compound; and (c) recovering the lysate composition from the sample, wherein the method further comprises contacting the sample with a nuclease inhibitor. In some embodiments, the sample may be contacted with a nuclease inhibitor prior to step (a). In certain preferred embodiment, the nuclease inhibitor may be an RNAse inhibitor. For example, the RNAse inhibitor may be selected from but is not limited to 2′-cytidine monophosphate free acid (2′-CMP), aluminon, adenosine 5′-pyrophosphate, 5′-diphosphoadenosine 3′-phosphate (ppA-3′-p), 5′-diphosphoadenosine 2′-phosphate (ppA-2′-p), Leucine, poly-L-aspartic acid, tyrosine-glutamic acid polymer, oligovinysulfonic acid, 5′-phospho-2′-deoxyuridine 3′-pyrophosphate P′→5′-ester with adenosine 3′-phosphate (pdUppAp).

Provided in another embodiment is a method for extracting a target chemical compound from a cellular material in a sample, the method comprising (a) subjecting the sample to mechanical lysis to cause disruption of a cellular membrane in the cellular material; (b) contacting the sample with an alkaline material to produce a lysate composition comprising the target chemical compound; and (c) recovering the lysate composition from the sample, wherein the method further comprises detecting at least one nucleotide sequence in the cell lysate. In some embodiments, one or more nucleotide sequence may be detected using a sandwich assay, preferably where the sandwich assay is conducted on an electrochemical sensor platform. In certain preferred embodiments, one or more nucleotide sequences may be detected by contacting the cell lysate with a capture probe. In other preferred embodiments, one or more nucleotide sequences may be detected by contacting the cell lysate with a magnetic bead, preferably where the magnetic bead comprises a capture probe or a detector probe. In certain preferred embodiments, the capture probe or detector probe may comprise one or more nucleic acids, examples of which may include but are not limited to DNA, peptide nucleic acids (PNAs), locked nucleic acids (LNAs) or any combination thereof. By way of non-limiting example, the capture probes and detector probes may each comprise 1, 2, 3, 4, 5, 6, 7, 8, 9, 10, 11, 12, 13, 14, 15, 16, 17, 18, 19, 20 or more nucleic acids. In further preferred embodiments, the detector probe may comprise a detectable label. By way of non-limiting example, the detectable label may be selected from a radionuclide, an enzymatic label, a chemiluminescent label, a hapten, and a fluorescent label. A fluorescent label for example, may be a fluorescent molecule selected from a fluorophore, a cyanine dye, and a near infrared (NIR) dye, or more preferably the fluorescent molecule may be fluorescein or fluorescein isothiocyanate (FITC). A hapten label may for example be selected from DCC, biotin, nitropyrazole, thiazolesulfonamide, benzofurazan, and 2-hydroxyquinoxaline.

In another of its aspects, the present invention provides a method for producing a lysate composition comprising RNA from a sample of mammalian origin comprising a cellular material, the method comprising the steps of: (a) rotating a microfluidic centrifugal disk comprising a lysis chamber containing the sample; (b) subjecting the sample to mechanical lysis to cause disruption of a cellular membrane in the cellular material; and (c) contacting the sample in the lysis chamber with an alkaline solution to produce the lysate composition.

Provided in one embodiment is a method for producing a lysate composition comprising RNA from a sample of mammalian origin comprising a cellular material, the method comprising the steps of: (a) rotating a microfluidic centrifugal disk comprising a lysis chamber containing the sample; (b) subjecting the sample to mechanical lysis to cause disruption of a cellular membrane in the cellular material; and (c) contacting the sample in the lysis chamber with an alkaline solution to produce the lysate composition, wherein the RNA may pre-ribosomal RNA, mature RNA, or may be selected from the group consisting of 16S rRNA, 23S rRNA or any mixture thereof.

Provided in another embodiment is a method for producing a lysate composition comprising RNA from a sample of mammalian origin comprising a cellular material, the method comprising the steps of: (a) rotating a microfluidic centrifugal disk comprising a lysis chamber containing the sample; (b) subjecting the sample to mechanical lysis to cause disruption of a cellular membrane in the cellular material; and (c) contacting the sample in the lysis chamber with an alkaline solution to produce the lysate composition, wherein the alkaline solution may comprise a sodium hydroxide solution. In certain preferred embodiments, the alkaline solution may have a concentration of about 10 M or less, preferably of about 1 M to 5M, and more preferably of about 1.5 M to 3M. In certain preferred embodiments, the alkaline solution may have a concentration of about 2M. In other preferred embodiments, the alkaline solution may have a concentration of about 3M.

Provided in another embodiment is a method for producing a lysate composition comprising RNA from a sample of mammalian origin comprising a cellular material, the method comprising the steps of: (a) rotating a microfluidic centrifugal disk comprising a lysis chamber containing the sample; (b) subjecting the sample to mechanical lysis to cause disruption of a cellular membrane in the cellular material; and (c) contacting the sample in the lysis chamber with an alkaline solution to produce the lysate composition, wherein the sample may comprise human cellular material, preferably a bodily fluid or an inoculant derived therefrom. In certain preferred embodiments, the bodily fluid may be selected from the group consisting of blood, urine, saliva, sweat, tears, mucus, breast milk, plasma, serum, synovial fluid, pleural fluid, lymph fluid, amniotic fluid, feces, cerebrospinal fluid and any mixture of two or more of these.

Provided in another embodiment is a method for producing a lysate composition comprising RNA from a sample of mammalian origin comprising a cellular material, the method comprising the steps of: (a) rotating a microfluidic centrifugal disk comprising a lysis chamber containing the sample; (b) subjecting the sample to mechanical lysis to cause disruption of a cellular membrane in the cellular material; and (c) contacting the sample in the lysis chamber with an alkaline solution to produce the lysate composition, wherein steps (a) and (b) may be conducted for a period of about 10 minutes or less, preferably from about 30 seconds to about 10 minutes, more preferably from about 1 minute to 8 minutes, and most preferably for a period of about 2 minutes±30 seconds, about 3 minutes±30 seconds, about 4 minutes±30 seconds, about 5 minutes±30 seconds, about 6 minutes±30 seconds, or about 7 minutes±30 seconds.

Provided in another embodiment is a method for producing a lysate composition comprising RNA from a sample of mammalian origin comprising a cellular material, the method comprising the steps of: (a) rotating a microfluidic centrifugal disk comprising a lysis chamber containing the sample; (b) subjecting the sample to mechanical lysis to cause disruption of a cellular membrane in the cellular material; and (c) contacting the sample in the lysis chamber with an alkaline solution to produce the lysate composition, wherein steps (a) and (b) may be carried out concurrently.

Provided in another embodiment is a method for producing a lysate composition comprising RNA from a sample of mammalian origin comprising a cellular material, the method comprising the steps of: (a) rotating a microfluidic centrifugal disk comprising a lysis chamber containing the sample; (b) subjecting the sample to mechanical lysis to cause disruption of a cellular membrane in the cellular material; and (c) contacting the sample in the lysis chamber with an alkaline solution to produce the lysate composition, wherein steps (b) and (c) may be carried out concurrently.

Provided in another embodiment is a method for producing a lysate composition comprising RNA from a sample of mammalian origin comprising a cellular material, the method comprising the steps of: (a) rotating a microfluidic centrifugal disk comprising a lysis chamber containing the sample; (b) subjecting the sample to mechanical lysis to cause disruption of a cellular membrane in the cellular material; and (c) contacting the sample in the lysis chamber with an alkaline solution to produce the lysate composition, wherein steps (b) and (c) may be carried out sequentially. In certain preferred embodiments, step (c) may be carried out after commencement of disruption of the cellular membrane in step (b).

Provided in another embodiment is a method for producing a lysate composition comprising RNA from a sample of mammalian origin comprising a cellular material, the method comprising the steps of: (a) rotating a microfluidic centrifugal disk comprising a lysis chamber containing the sample; (b) subjecting the sample to mechanical lysis to cause disruption of a cellular membrane in the cellular material; and (c) contacting the sample in the lysis chamber with an alkaline solution to produce the lysate composition, wherein the mechanical lysis may comprise a combination of centrifugation and puck lysing. In some embodiments, the puck lysing may be magnetic puck lysing. In certain preferred embodiments, the combination of centrifugation and puck lysing may be carried out in a common lysis chamber, preferably centrifugation and puck lysing may be carried out on a centrifugal disk.

In yet another of its aspects, the present invention provides a method for extracting a nucleic acid from a cellular material in a sample comprising a bodily fluid or an inoculant derived therefrom, the method comprising the steps of (a) subjecting the sample to a first lysing process comprising mechanical lysis to cause disruption of a cellular membrane in the cellular material; (b) subjecting the sample to a second lysing process comprising at least one of physical lysis, chemical lysis, biological lysis and any combination of two or more of these to produce a lysate composition comprising the nucleic acid; and (c) recovering the lysate composition from the sample.

Provided in one embodiment is a method for extracting a nucleic acid from a cellular material in a sample comprising a bodily fluid or an inoculant derived therefrom, the method comprising the steps of (a) subjecting the sample to a first lysing process comprising mechanical lysis to cause disruption of a cellular membrane in the cellular material; (b) subjecting the sample to a second lysing process comprising at least one of physical lysis, chemical lysis, biological lysis and any combination of two or more of these to produce a lysate composition comprising the nucleic acid; and (c) recovering the lysate composition from the sample, wherein the nucleic acid may be deoxyribonucleic acid (DNA) or ribonucleic acid (RNA). In certain preferred embodiments, the nucleic acid may be ribosomal RNA, or more preferably may pre-ribosomal RNA, mature RNA, or may be selected from the group consisting of 16S rRNA, 23S rRNA or any mixture thereof.

Provided in another embodiment is a method for extracting a nucleic acid from a cellular material in a sample comprising a bodily fluid or an inoculant derived therefrom, the method comprising the steps of (a) subjecting the sample to a first lysing process comprising mechanical lysis to cause disruption of a cellular membrane in the cellular material; (b) subjecting the sample to a second lysing process comprising at least one of physical lysis, chemical lysis, biological lysis and any combination of two or more of these to produce a lysate composition comprising the nucleic acid; and (c) recovering the lysate composition from the sample, wherein the chemical lysis may comprise contacting the sample with an alkaline solution. In some embodiments, the alkaline solution may comprise a sodium hydroxide solution. In certain preferred embodiments, the alkaline solution may have a concentration of about 10 M or less, preferably of about 1 M to 5M, and more preferably of about 1.5 M to 3M. In certain preferred embodiments, the alkaline solution may have a concentration of about 2M. In other preferred embodiments, the alkaline solution may have a concentration of about 3M.

Provided in another embodiment is a method for extracting a nucleic acid from a cellular material in a sample comprising a bodily fluid or an inoculant derived therefrom, the method comprising the steps of (a) subjecting the sample to a first lysing process comprising mechanical lysis to cause disruption of a cellular membrane in the cellular material; (b) subjecting the sample to a second lysing process comprising at least one of physical lysis, chemical lysis, biological lysis and any combination of two or more of these to produce a lysate composition comprising the nucleic acid; and (c) recovering the lysate composition from the sample, wherein the bodily fluid may comprise human cellular material, and more preferably may be selected from the group consisting of blood, urine, saliva, sweat, tears, mucus, breast milk, plasma, serum, synovial fluid, pleural fluid, lymph fluid, amniotic fluid, feces, cerebrospinal fluid and any mixture of two or more of these.

Provided in another embodiment is a method for extracting a nucleic acid from a cellular material in a sample comprising a bodily fluid or an inoculant derived therefrom, the method comprising the steps of (a) subjecting the sample to a first lysing process comprising mechanical lysis to cause disruption of a cellular membrane in the cellular material; (b) subjecting the sample to a second lysing process comprising at least one of physical lysis, chemical lysis, biological lysis and any combination of two or more of these to produce a lysate composition comprising the nucleic acid; and (c) recovering the lysate composition from the sample, wherein step (a) may be conducted for a period of about 10 minutes or less, preferably from about 30 seconds to about 10 minutes, more preferably from about 1 minute to 8 minutes, and most preferably for a period of about 2 minutes±30 seconds, about 3 minutes±30 seconds, about 4 minutes±30 seconds, about 5 minutes±30 seconds, about 6 minutes±30 seconds, or about 7 minutes±30 seconds.

Provided in another embodiment is a method for extracting a nucleic acid from a cellular material in a sample comprising a bodily fluid or an inoculant derived therefrom, the method comprising the steps of (a) subjecting the sample to a first lysing process comprising mechanical lysis to cause disruption of a cellular membrane in the cellular material; (b) subjecting the sample to a second lysing process comprising at least one of physical lysis, chemical lysis, biological lysis and any combination of two or more of these to produce a lysate composition comprising the nucleic acid; and (c) recovering the lysate composition from the sample, wherein the mechanical lysis may comprise a combination of centrifugation and puck lysing. In some embodiments, the puck lysing may be magnetic puck lysing. In certain preferred embodiments, the combination of centrifugation and puck lysing may be carried out in a common lysis chamber, preferably centrifugation and puck lysing may be carried out on a centrifugal disk.

Provided in another embodiment is a method for extracting a nucleic acid from a cellular material in a sample comprising a bodily fluid or an inoculant derived therefrom, the method comprising the steps of (a) subjecting the sample to a first lysing process comprising mechanical lysis to cause disruption of a cellular membrane in the cellular material; (b) subjecting the sample to a second lysing process comprising at least one of physical lysis, chemical lysis, biological lysis and any combination of two or more of these to produce a lysate composition comprising the nucleic acid; and (c) recovering the lysate composition from the sample, wherein steps (a) and (b) may be carried out concurrently.

Provided in another embodiment is a method for extracting a nucleic acid from a cellular material in a sample comprising a bodily fluid or an inoculant derived therefrom, the method comprising the steps of (a) subjecting the sample to a first lysing process comprising mechanical lysis to cause disruption of a cellular membrane in the cellular material; (b) subjecting the sample to a second lysing process comprising at least one of physical lysis, chemical lysis, biological lysis and any combination of two or more of these to produce a lysate composition comprising the nucleic acid; and (c) recovering the lysate composition from the sample, wherein steps (a) and (b) may be carried out sequentially. In certain preferred embodiments, step (b) may be carried out after commencement of disruption of the cellular membrane in step (a).

The methods disclose herein may comprise performing one or more mechanical lyses and one or more non-mechanical lyses.

In some embodiments, lysing the microorganism occurs prior to determining the quantity of a nucleic acid molecule in a plurality of inoculates.

In some embodiments, the methods disclosed herein comprise contacting the neutralized cell lysate with a solution comprising streptavidin.

### Nucleic Acid Molecule Quantification

In some embodiments, the methods disclosed herein comprise detecting the quantity of a nucleic acid molecule from a microorganism in a sample. In some embodiments, the methods disclosed herein comprise comparing the quantity of a nucleic acid molecule in the antimicrobial agent-free inoculate to the quantity of a nucleic acid molecule in the antimicrobial agent inoculate. In some embodiments, the nucleic acid molecule is a deoxyribonucleic acid (DNA), ribonucleic acid (RNA), or a combination thereof.

In some embodiments, the methods disclosed herein are a RiboResponse™ method. In some embodiments, the RiboResponse™ method comprises determining the quantity of an RNA molecule from the microorganism. In some embodiments, the RNA is a mature RNA. In some embodiments, the RNA is a precursor RNA. In some embodiments, the RNA is a ribosomal RNA (rRNA). In some embodiments, the rRNA is a 16S RNA or 23S RNA. In some embodiments, the microorganism is a prokaryote. In some embodiments, the prokaryote is a Gram-negative bacterium. In some embodiments, the prokaryote is a Gram-positive bacterium. In some embodiments, the microorganism is fungal (e.g., candida).

The RiboResponse™ platform is quantitative in that more bacteria would result in more ribosomes and, hence, ribosomal RNA, resulting in a higher detection signal when ribosomal RNA is detected. In some embodiments, the detected level of a nucleic acid molecule in each of the plurality of inoculates comprising an antimicrobial agent for each antibiotic is compared to the control lacking an anti-microbial agent (ideal growth) and expressed as a percentage of the no antibiotic control. In some embodiments, resistant antibiotics have numbers close to 100%, meaning they had a comparable level of growth to the no antibiotic control. In some embodiments, an inoculate with an antimicrobial agent to which a microorganism is susceptible antibiotics will have a nucleic acid molecule detection level lower than 100%, e.g., 90%, 85%, 80%, 75%, 70%, 65%, 60%, 55%, 50%, 45%, 40%, 35%, 30%, 25%, 20%, 15%, 10%, or 5%, or less.

In some embodiments, determining the quantity of a nucleic acid molecule in an inoculate allows for an estimation of bacterial density (or quantity) in the inoculate. In some embodiments, the density of bacteria in a sample (or a particular inoculate) is estimated by applying a formula that contains variables m (slope) and b (y-intercept) derived from an empirically determined species-specific standard curve. The bacterial assay signal is normalized by dividing it by the assay positive control signal and multiplying the result by 1000. Where y=log₁₀ (normalized assay signal), the formula that relates the assay signal to CFU/mL is:

$${log}_{10} ( CFU / ml ) = \frac{( y - b )}{m}$$

In some embodiments, a predicted CFU/mL value is multiplied by an adjustment factor Fa to provide an improved estimate of final bacterial density, where Fa is based on comparing formula predictions to observed inoculation results:

$${F}_{a} = 1 + 5.76 \times {{e}^{- 0.5} [ \frac{{log}_{10} CFU / ml - 7.23}{0.259} ]}^{2}$$

In some embodiments, the plurality of inoculates is serially diluted in cell culture media prior to quantification of a nucleic acid molecule.

In some embodiments, determining the quantity of a nucleic acid molecule in a plurality of inoculates comprises a sandwich assay. In some embodiments, determining the quantity of a nucleic acid molecule in a plurality of inoculates comprises using an electrochemical sensor platform. In some embodiments, determining the quantity of a nucleic acid molecule in a plurality of inoculates comprises using an ELISA. In some embodiments, determining the quantity of a nucleic acid molecule in a plurality of inoculates comprises using a magnetic bead-based detection platform.

### Antimicrobial Agent Susceptibility

Disclosed herein are methods for determining the susceptibility of a microorganism to an antimicrobial agent. In some embodiments, the microorganism is susceptible to the antimicrobial agent if the quantity of nucleic acid molecules of the microorganism in the antimicrobial agent-free inoculate is more than the quantity of nucleic acid molecules of the microorganism in an inoculate comprising the microorganism and the antimicrobial agent. In some embodiments, the microorganism is not susceptible to the antimicrobial agent if the quantity of nucleic acid molecules of the microorganism in the antimicrobial agent-free inoculate is nearly equal, equal, or less than the quantity of nucleic acid molecules of the microorganism in an inoculate comprising the microorganism and the antimicrobial agent.

### Reports and Data Transmission

In some embodiments, the methods disclosed herein further comprise generating one or more reports. In some embodiments, the methods disclosed herein further comprise transmitting one or more reports. In some embodiments, the report includes information on the susceptibility of a microorganism to one or more antimicrobial agents or combinations of antimicrobial agents. In some embodiments, the report provides recommendations on a therapeutic regimen. In some embodiments, the report provides recommendations on the dosage of an antimicrobial agent.

### Detecting the Presence of Microorganisms in a Clinical Specimen

When using some of the methods described herein, the quantity of the microorganism in the sample to be tested may be known. For example, a sample may be prepared for the purpose of undergoing the AST techniques described herein, and one or more of its parameters may be prescribed as part of the test procedure. This may be the case in research or laboratory testing environments. In other circumstances, at least some of the properties of the sample that is to be subjected to the AST analysis are very likely to be unknown to those performing the test. For example, if a clinical specimen is taken from a patient for analysis, the nature of the microorganisms (if any) and their quantity within the sample may be unknown. Without at least some type of approximate or “good enough” estimation of the quantity of the microorganism (e.g. bacteria) in the sample it may be relatively more difficult to calibrate the inputs of a desired AST process. For example, it may be more difficult to determine if a sample ought to be diluted, and if so by what extent, and to select an appropriate dosage(s) of the one or more antimicrobial agents that may be used in the process. That is, quantification of bacterial density may, for example, be useful in determining the correct inoculation of a clinical specimen into growth medium for the AST. It may also be useful in determining if an infection is present or not.

To help reduce this uncertainty, particularly when desiring to test samples of unknown contents, it may, in some circumstances, be desirable to quantify the bacterial density in a clinical specimen prior to conducting the AST.

Some examples of suitable methods for quantify the bacterial density in a clinical specimen have been disclosed in provisional patent application S.N. 62/671,380, filed May 14, 2018, the contents of which are hereby incorporated by reference herein in its entirety.

In some embodiments, the methods disclosed herein further comprise steps of quantifying bacterial density in a clinical specimen. Such quantification is based on the following Translation Function:

[rRNA]=f(z)·cfu/ml

where z is the number of rRNA copies per cell, [rRNA] is the bacterial rRNA concentration, and CFU/mL is the bacterial density in a bacteria-containing specimen.

As indicated by the above equation, the number of rRNA copies per cell (z) may be a linear function, which may be at least partially dependent on bacterial concentration. FIG. 22 shows an equation that relates rRNA copies per cell to bacterial concentration in urine specimens.

In accordance with one aspect of the teachings described herein, a method of in a urine specimen of a patient with a urinary tract infection (UTI) is described. FIG. 20 is a flowchart illustrating one embodiment of this method.

FIG. 20 sets out one example of a method 100 of estimating the bacterial density in a clinical specimen. This method includes a first step 102 of obtaining a clinical specimen. In most embodiments of the method, the clinical specimen is believed to contain at least one species of bacteria in a clinically relevant amount, and may be suspected of containing two or more species of bacteria in a clinically relevant amount. In the illustrated example, the clinical specimen is a urine specimen obtained from a patient that is complaining of symptoms consistent with a urinary tract infection and the specimen is suspected of containing at least a clinically relevant amount of E. coli.

Once the specimen suspected of containing a clinically relevant amount of bacteria is obtained, in a second step 104, the rRNA of the bacteria in the specimen is processed to obtain an rRNA signal. At least one positive control and at least one negative control are included in step 104.

In some embodiments of the invention, the time it takes from when a clinical specimen is obtained (i.e. step 102) to when the rRNA of at least one bacterial species in the clinical specimen has been processed (i.e. step 104) is less than four (4) hours. In some preferred embodiments, the time it takes from when a clinical specimen is obtained (i.e. step 102) to when the rRNA of at least one bacterial species in the clinical specimen has been processed (i.e. step 104) is less than 3 hours; less than 2 hours; less than 1 hour; less than 30 minutes; or less than 15 minutes.

The rRNA signal obtained from step 104 may then be used to determine the rRNA concentration of the bacteria in the specimen, preferably automatically when using a suitable system (i.e. without requiring intervention from a skilled technician). A determination of rRNA concentration may be based on a linear log-log correlation between the assay signal and the concentration of the rRNA analyte. Therefore, in a next step 106, the log of the rRNA signal from step 104 may be calculated to give the rRNA signal_{LOG}.

In a next step 108, the log of the negative control signal from step 104 is subtracted from both the rRNA signal_{LOG} from step 106 and the log of the positive control signal from step 104. The resulting rRNA signal_{LOG} is then compared with the resulting positive control signal_{LOG} to normalize the signal intensity of the rRNA signal_{LOG} and determine the rRNA concentration of bacteria in the clinical specimen (units=pM, Log10).

In a next step 110, the rRNA concentration from step 108 may be inputted into a predetermined translation function to estimate the bacterial density value_{LOG} in the clinical sample (units=CFU/ml, Log10).

In a next step 112, the inverse log of the bacterial density value_{LOG} from step 110 may be calculated to estimate the bacterial density value of the clinical specimen (units=CFU/ml).

### RNA Quantification (Step 104)

Determining the concentration of rRNA may be done using any suitable method, including those described herein. One example of a suitable method may include the steps of: 1) Lysis to release rRNA 128; 2) Neutralization 130; 3) Hybridization of target rRNA with a capture probe and detector probe 132; and 4) Detection of capture probe—target rRNA—detector probe complexes 134.

Optionally, the method of determining the concentration of the rRNA may be performed at least partially, and preferably completely, automatically using a suitable apparatus.

In the illustrated example, a MagPix (Luminex) magnetic bead assay is used to measure the E. coli rRNA concentration in fresh urine specimens from a patient with UTI.

### Lysis (Step 128)

Optionally, the lysing step 128 may include at least one of chemical lysing, mechanical lysing, and/or a combination thereof. In a preferred embodiment, lysis 128 may include both chemical and mechanical lysing operations, as described above and in PCT/US18/45211, which is incorporated herein by reference herein in its entirety.

### Neutralization (Step 130)

The neutralization step 130 can be performed using any known or unknown method.

In the illustrated example, samples are lysed with one-half volume of 1 M NaOH. This lysate is neutralized with an equal volume of 1 M sodium-potassium phosphate buffer, pH 6.4.

### Hybridization (Step 132)

Preferably, a species-specific signal can be provided for each type of target bacteria that is expected to be present in the clinical specimen. By using a species-specific signal, the signal of rRNA from different types of bacteria in mixed specimens may be individually observed/counted and/or only signals from the desired, targeted bacteria may be counted. This may help facilitate the quantification of two or more different target bacteria within a common clinical specimen, and may allow the concentrations of two or more target bacterial rRNA concentrations to be measured generally simultaneously.

This may be advantageous when analyzing certain types of clinical specimens, such as urine specimens, which may tend to include a variety of different bacteria in generally unknown quantities at the beginning of the analysis process. By using species-specific signal probes, the methods described herein could be used to independently determine a quantity of rRNA from two or more specific bacterial species in the clinical specimen, input those values into respective, pre-determined transfer functions and calculate respective rRNA concentration values for each bacterial species. These results can then be used to provide outputs and/or as inputs in other method steps on a species-specific basis. For example, the methods may indicate a bacterial density value for E. coli that is above an E. coli pre-determined treatment threshold, while a bacterial density value for K. pneumoniae is below its respective pre-determined treatment threshold. This may be used to initiate further treatment or diagnoses methods regarding E. coli, while not initiating analogous steps for K. pneumoniae. Alternatively, if both bacterial density values are above their respective pre-determined treatment thresholds, a different, suitable treatment protocol may be selected or followed.

### Detection (Step 134)

A variety of platforms can be used for detection 134, including but not limited to excitation and imaging of fluorescent-tagged detector probes, bioluminescence using luciferase-type enzymes, and amperometric current using an electrochemical sensor. In the illustrated example, fluorescent-tagged detector probes are used for detection.

During detection 134, at least one positive control and at least one negative control are included. In the illustrated example, a synthetic oligonucleotide with the same sequence as the target rRNA is included as a positive control and a sample without rRNA or bacteria is included as a negative control.

### Translation Function

The translation function used in step 110 is preferably selected from amongst one or more pre-determined translation functions. Suitable translation functions may be determined using any suitable technique, including those described herein. Optionally, more than one translation function may be determined and may be stored or otherwise recorded in a translation function table. For example, different translation functions may be developed for different species of bacteria that may be expected to be present in an incoming clinical specimen. That is, one translation function may be used to correlate the rRNA concentration and CFU/mL of E. coli in a given specimen, while a different translation function may be used to correlate the concentration of rRNA and CFU/mL of K. pneumoniae. Some translation functions may be better suited for use with a given type of bacteria.

Each translation function may take as an input a value that is based on the species-specific rRNA concentration in the specimen. For example, a translation function derived for E. coli may take as its input a value corresponding to the rRNA concentration of E. coli in the specimen, whereas a translation function for K. pneumoniae may take as its input a value corresponding to the rRNA concentration of K. pneumoniae in the specimen.

If more than one translation function has been determined, the methods and/or systems described herein may include the steps of selecting one translation function, from the two or more translation functions available, as being most appropriate for use with a given clinical specimen. The selection of a given translation function may be based on a variety of factors, including user inputs/selections, the expected types of bacteria, the type of specimen, ambient temperature, and sample storage time.

In a preferred embodiment, a translation function is derived from a bacterial species-specific standard curve. To derive a bacterial species-specific standard curve, rRNA concentrations of a specific bacteria may be measured in a group of clinical specimens of the same type (e.g. a group of urine specimens). Species-specific bacterial densities may then be determined on the same specimens using any known method. This relationship may then be plotted on a graph, with rRNA concentration (pM, Log10) on one axis and CFU/mL (Log10) on the other axis to determine the correlation between rRNA concentration and bacterial density. The resulting relationship between these two variables may define a translation function.

The number of specimens required to derive a bacterial species-specific standard curve may depend on such factors as the type of specimen and the species of bacteria being analyzed. The number of specimens required to accurately define a relationship between rRNA concentration and bacterial density may be determined using known statistical methods.

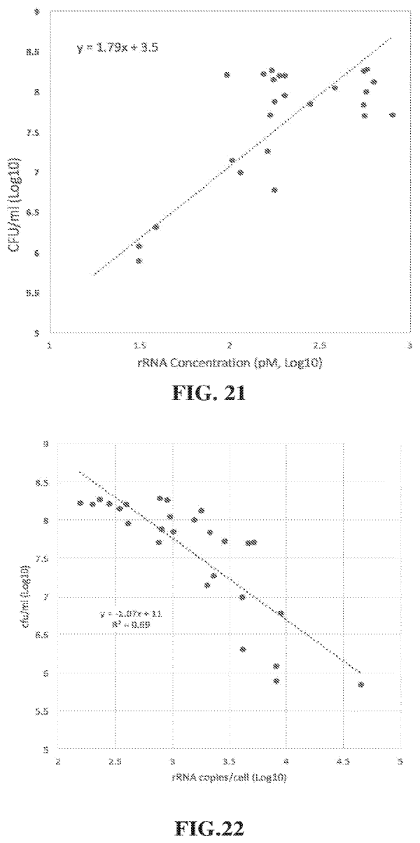

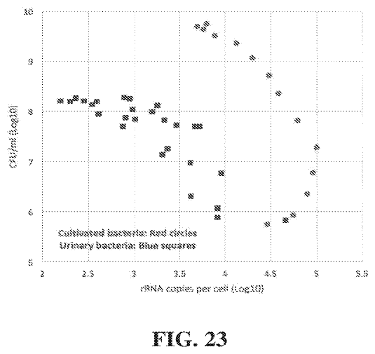

In the illustrated example, in a first step 136, a MagPix (Luminex) magnetic bead assay is used to measure E. coli rRNA concentrations in fresh urine specimens from 25 patients with UTI, as according to steps 102-108. In a next step 138, the bacterial density of E. coli in each specimen is determined with plate counts. In a next step 140, the log of each bacterial density from step 138 is calculated for each specimen to obtain the bacterial density_{LOG}, which, in a next step 142, is plotted on a scatterplot against the rRNA concentration from step 136. From this scatterplot, the correlation between rRNA concentration and bacterial density is determined. FIG. 21 illustrates the correlation between E. coli rRNA concentration and density of E. coli for urine specimens from 25 patients with E. coli urinary tract infection.

In the illustrated example, the slope of the resulting regression line may be used as the translation function to estimate the E. coli bacterial density value (CFU/ml) in a urine specimen. More specifically, the linear equation of the resulting regression line, as represented by the general formula y=mx+b, may be used to estimate the bacterial density value (CFU/ml) of E. coli in a clinical specimen, wherein x is the rRNA concentration of E. coli in a clinical specimen (pM, Log10) and y is the bacterial density value of E. coli in the clinical specimen (CFU/ml, Log10).

In the illustrated example, the linear equation of the resulting regression line, and therefore the translation function, is y=1.79x+3.5, as seen in FIG. 21. Therefore, in the illustrated example, the translation function for E. coli was empirically determined to be y=1.79x+3.5, where y is CFU/mL (log 10) and x is the rRNA concentration (pM, Log 10) value for the tested clinical specimen. The bacterial density value in units of CFU/mL can then be obtained by taking the inverse log of y. In other words, the bacterial density value for E. coli can be described as:

bacterial density value=antilog (1.79x+3.5)

While in this example the x coefficient is presented with three significant digits, other examples of the translation function may have only a single decimal point or may be otherwise rounded while still providing a sufficiently accurate output for the bacterial density value on which to base clinical decisions.

### Bacterial Density Value

Optionally, the bacterial density value (from step 112) can be provided to a user, for example via any suitable type of user display apparatus, such as a screen, print-out, email, text message, graphic, or the like. This information may then be used for any suitable purpose, including, for example, reporting and/or regulatory compliance.

In some embodiments, the bacterial density value may be used as an input or otherwise implicated in other sorts of methods. For example, in one embodiment, the bacterial density value may be used to determine the likelihood of infection. In another embodiment, the bacterial density value may be used as one of the inputs in a method or process that is to be performed on the clinical specimen. In another embodiment, the bacterial density value may be used as a predictor of wound healing and/or acceptance of grafts.

### Screening for Infection

Quantification of bacterial density may be useful in testing clinical specimens for the presence of bacteria above a certain predetermined cutoff or threshold. Bacterial densities above the cutoff may be considered positive and indicate the presence of infection; bacterial densities below the cutoff may be considered negative and may indicate such factors as contamination of the specimen during collection or outgrowth of contaminants during storage or transport.

In the illustrated example, at step 144, a false negative rate of <5% is determined to be sufficient to assess the likelihood of infection in a clinical specimen. This means that the cutoff for the assessment of infection is set to 2 standard deviations above background, meaning that if the bacterial density value of a specimen is greater than or equal to 2 standard deviations above background, there is a likelihood of infection. Conversely, if the bacterial density value of a specimen is less than 2 standard deviations above background, there is not a likelihood of infection.

In the illustrated example, the likelihood of infection in a clinical specimen is assessed in steps 114-118. As a first step 114, the bacterial density value of E. coli in a urine specimen (from step 112) is compared with the predetermined infection threshold of 2 standard deviations above background (from step 144). If the bacterial density value from step 112 is greater than or equal to the infection threshold (i.e. >2 standard deviations above background), a positive output indicating the likelihood of infection is produced, as seen at step 116. Alternatively, if the bacterial density value from step 112 is less than the infection threshold (i.e. <2 standard deviations above background), a negative output indicating that infection is not likely is produced, as seen at step 118.

### AST Inoculation Concentration

Quantification of bacterial density may be useful in determining the correct inoculation of a clinical specimen into growth medium for a direct from specimen phenotypic AST. Providing a bacterial density value that is within an acceptable resolution for clinical analysis may help determine an appropriate dosage of an inoculation agent to be used with a given clinical specimen to help provide a desired or target inoculation concentration in the clinical specimen. Utilizing the bacterial density value as a factor to help determine the dosage of the inoculation may help reduce the likelihood of over or under-diluting a given clinical specimen during further processing.

For example, in one embodiment, the target inoculation concentration of the AST may be 5×10⁵ CFU/ml. Inoculation concentrations up to 5×10⁶ CFU/mL may provide an accurate AST result, whereas inoculation concentrations greater than 5×10⁶ CFU/mL may limit growth, thereby possibly reducing accuracy of AST results.

In the illustrated example, the determination of the AST inoculation concentration of the clinical specimen is set out in steps 120-126. As a first step, the bacterial density value from step 112 is compared to the predetermined desired target inoculation concentration for AST. If the bacterial density value from step 112 is greater than the desired target inoculation concentration, step 122 is engaged, in which the dilution factor required to dilute the bacterial density value of the specimen to within the desired target inoculation concentration range is determined. Based on the calculated dilution factor from step 122, growth medium is added to dilute the specimen to within the desired target range, as per step 124. The specimen can then be inoculated into growth medium for the AST, as per step 126.

On the other hand, if the bacterial density value from step 112 is less than or equal to the desired target inoculation concentration, the specimen may be inoculated into growth medium for the AST without dilution. In other words, steps 122-124 may be by-passed and the user would go immediately to step 126.

### Automation

Preferably, some or all of the steps in the methods can be automated using suitable equipment and do not require a skilled laboratory technician or the like to process the specimens and/or interpret the results. In some embodiments described herein, the inputs for the analysis method is a generally “fresh”, unmodified specimen obtained directly from a subject and the output of the method is an answer that is usable and/or understandable by a lay operator (i.e. not a skilled lab technician). For example, the output may be in the form of a number that represents the concentration of the target bacteria within the specimen.

### Experimental Examples

Embodiments of the present invention will now be illustrated with reference to the following examples which should not be used to construe or limit the scope of the present invention.

### A. Determining the Susceptibility of Bacteria to Antibiotics Using a Riboresponse™ Method

In this example, the materials and methods for performing a RiboResponse™ method for determining the susceptibility of bacteria to a plurality of antibiotics are provided.

Materials
1. Detector probe buffer: Mixture of detector probes (100 nM) in 1 M Phosphate Buffer pH 6.4.
2. Bead plate: 96-well plate containing Luminex MTAG beads functionalized with capture probes.
3. Positive control: 100 pM synthetic target in 1 M Phosphate Buffer pH 6.4.
4. AST plate: 96-well plate containing 180 μl of Cation-adjusted Mueller Hinton (MH2) broth per well, containing the working concentration of the appropriate antibiotic in the appropriate wells. To this plate is applied a 96-well plate sticker to prevent cross-contamination and evaporation.
5. Lysis plate: 96-well plate containing 25 μl of 1 M NaOH per well.
6. 1×Tm HB=0.1 M Tris pH 8.0, 0.2 M NaCl, 0.08% Triton X-100
7. 1 M NaOH
8. Streptavidin-phycoerythrin conjugate

Equipment
1. Shaker Incubator
2. Biotek 405TS Plate Washer
3. Luminex MagPix Assay System

Set 1 of Antibiotic Concentrations (working concentrations):
1. Gentamicin 4 μg/ml
2. Ciprofloxacin 4 μg/ml
3. Cefazolin 64 μg/ml
4. Ceftriaxone 32 μg/ml
5. Cefepime 64 μg/ml
6. Ampicillin 512 μg/ml
7. Imipenem 4 μg/ml
8. Trimethoprim 8 μg/ml and Sulfamethoxazole 152 μg/ml
9. Amikacin 64 μg/ml
10. Nitrofurantoin 16 μg/ml
11. Fosfomycin 64 μg/ml
12. Piperacillin 16 μg/ml and Tazobactam 4 μg/ml
13. Amoxicillin 32 and Clavulanate 16

Set 2 of Antibiotic Concentrations (working concentrations):
1. Gentamicin 4 μg/ml and 2 μg/ml
2. Ciprofloxacin 4 μg/ml
3. Cefazolin 64 μg/ml
4. Ceftriaxone 32 μg/ml
5. Cefepime 64 μg/ml and 32 μg/ml
6. Ampicillin 128 μg/ml
7. Trimethoprim 4 μg/mL and Sulfamethoxazole 76 μg/ml
8. Amikacin 32 μg/ml, 16 μg/ml and 8 μg/ml
9. Nitrofurantoin 16 μg/ml
10. Fosfomycin 64 μg/ml
11. Amoxicillin 64 μg/mL and Clavulanate 32 μg/ml; Amoxicillin 32 μg/mL and Clavulanate 16 μg/ml; Amoxicillin 16 μg/mL and Clavulanate 8 μg/ml
12. Etrapenem 4 μg/ml and 2 μg/ml
13. Meropenem 4 μg/ml and 2 μg/ml

Method 1: RiboResponse™ Method Using a Microtiter Plate
1. The AST plate was prewarmed and aerated by shaking in the 37° C. shaker incubator at 400 rpm.
2. The specimen was adjusted to a concentration of ˜5×10⁶ cfu/ml.
3. The wells of the 96-well AST plate were inoculated by adding 20 μl to the 180 μl in the well to yield 5×10⁵ CFU/ml. Uninoculated wells were included for negative and positive controls.
4. After inoculation, a 50 μl sample was transferred from the 0 min No Abx well to the corresponding well in the lysis plate containing 25 μl 1 M NaOH and mixed by pipetting.
5. The inoculated 96-well plate was placed in the 37° C. shaking incubator at 400 rpm for 90-120 minutes.
6. After 5 minutes of incubation at room temperature, 75 μl of detector probe buffer was added to neutralize the lysate and mixed by pipetting.
7. Steps 5 and 6 were repeated for the other wells in the AST and Lysis plates at the end of the 90-120 minute incubation period.
8. The negative control was neutralized in the same way and 100 pM synthetic target in detector probe buffer was used for the positive control.
9. The bead plate was shaken using the 2 minute fast shaking cycle on the Biotek Plate washer
10. The beads were washed in the Biotek plate washer using the Biotek Bead Washing Protocol below.
11. The multichannel pipettor was used to add 25 μl of the bead capture probe mixture from the 96-well bead plate to each well in the lysis plate.
12. The plate was shaken (without magnet) for 15 minutes on the variable setting with the Biotek plate washer.
13. The beads were washed in the Biotek plate washer using the Biotek Bead Washing Protocol below.
14. While the plate was washing, 2 μl of 1 mg/mL Streptavidin-PE stock was added to 1000 μl 1×Tm HB to yield 2 μg/ml.
15. After the plate has finished washing, 75 μl 2 μg/mL Streptavidin-PE was added to the appropriate wells.
16. The plate was shaken on variable speed with the Biotek plate washer for 1 minute.
17. The beads were washed with the Biotek plate washer following the protocol listed below.
18. The beads were measured in the Luminex MagPix instrument.

Method 2: Riboresponse™ Method Using a Centrifugal Disc

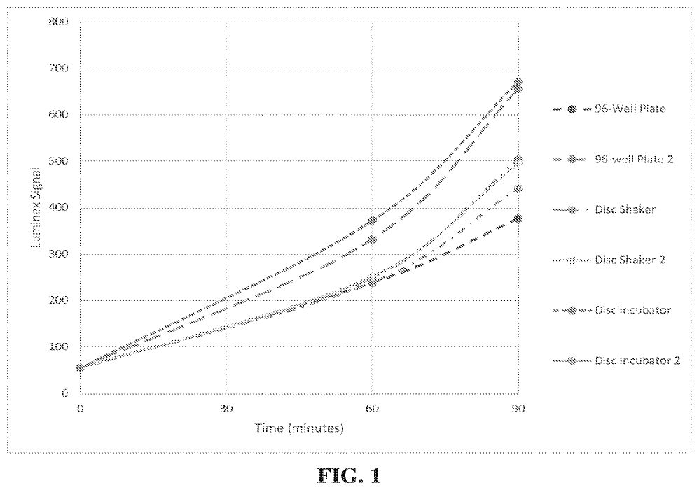

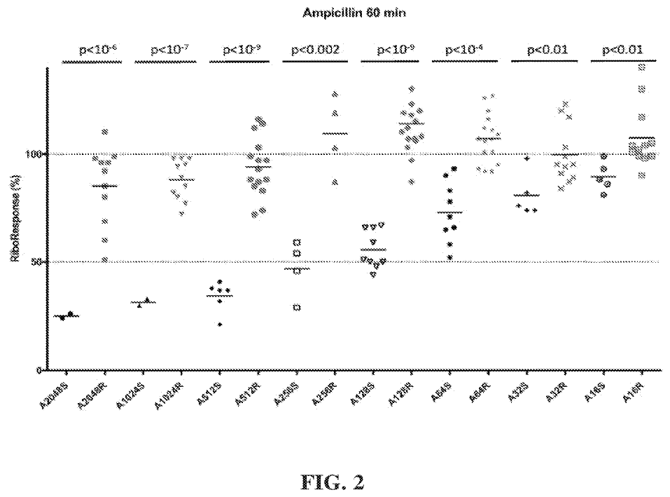

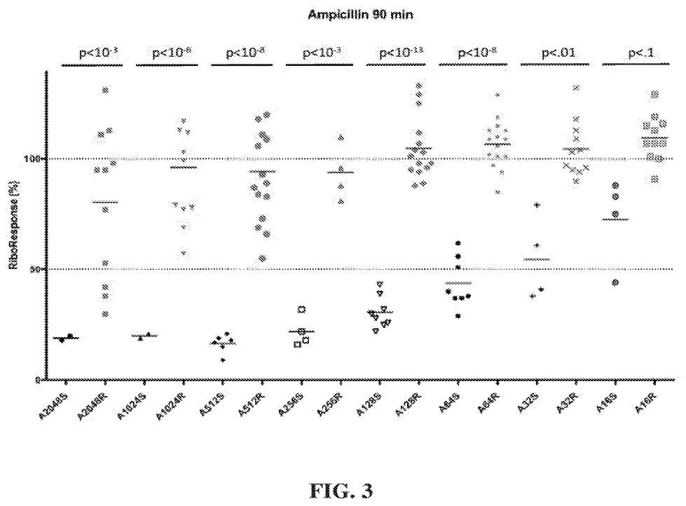

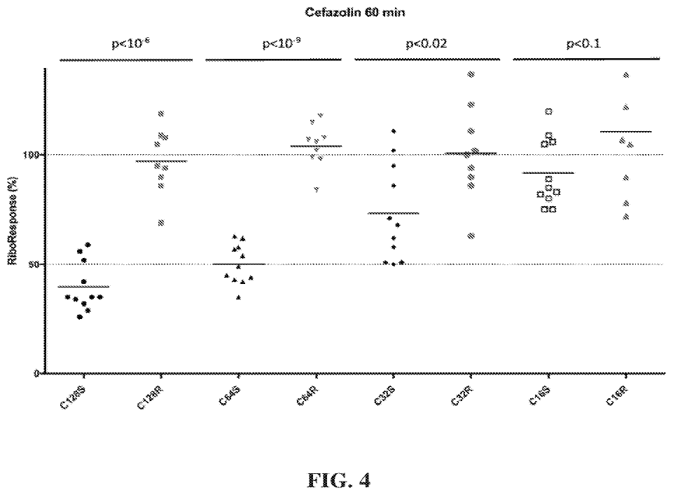

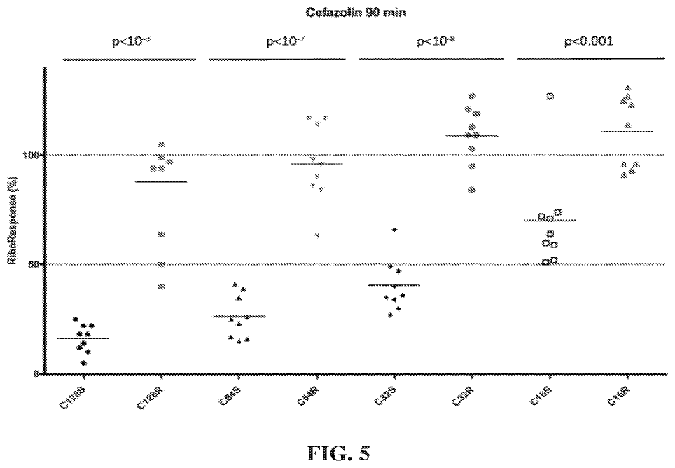

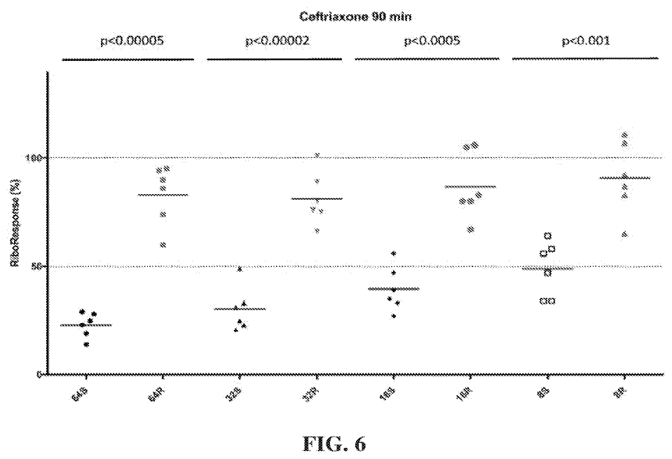

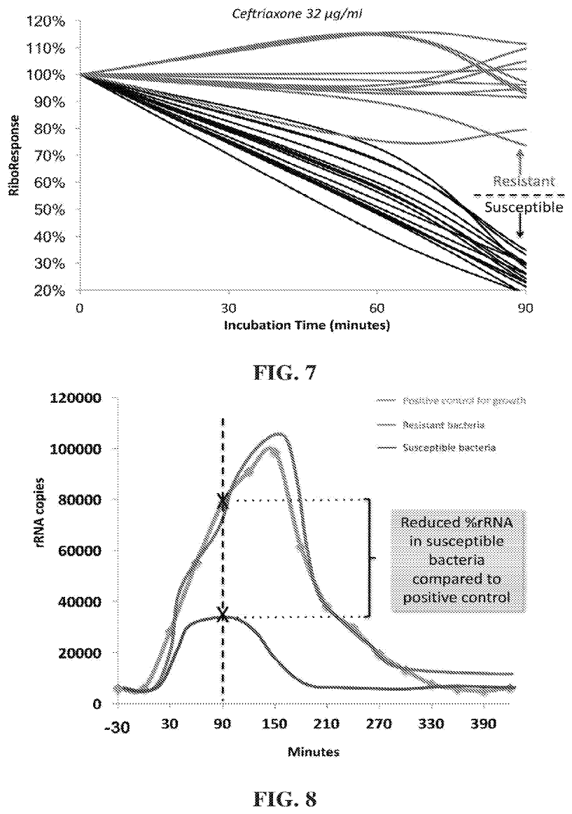

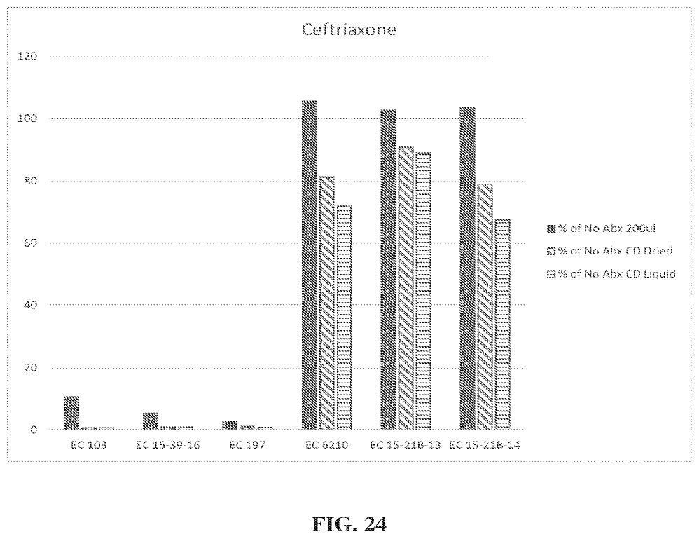

The RiboResponse™ method using a centrifugal disc is similar to Method 1 (above), except that the 90-120-minute incubation was performed in incubation chambers of a centrifugal disc. As shown in FIG. 1, growth in a rotating centrifugal disc was significantly faster than growth in a shaking centrifugal disc or shaking 96-well plate. Accelerated growth enables faster separation of susceptible and resistant bacteria. FIG. 24 is another example, illustrating enhanced results when incubation was conducted on a centrifugal disc.

Biotek Bead Washing Protocol (using 96-well plate magnet)
1. Shake on medium for 30 seconds
2. Soak for 30 seconds
3. Aspirate
4. Dispense 200 μl of 1×Tm HB per well
5. Shake on medium for 30 seconds
6. Soak for 30 seconds
7. Aspirate
8. Dispense 200 μl of 1×Tm HB per well
9. Shake on medium for 30 seconds
10. Soak for 30 seconds
11. Aspirate
12. FINAL WASH ONLY: Dispense 50 μl

Biotek 96 well plate washer settings:
Aspirate options—Z=43 (5.46 mm above carrier), X=30 (1.37 mm right of center)
Dispense options—Z=130 (16.52 mm above carrier), X=0
Slow mixing→7 Hz (420 rpm)
Medium mixing→13 Hz (780 rpm)
Fast mixing→19 Hz (1140 rpm)
Variable mixing→repeated cycle of (slow, medium, and fast mixing)×∞, cycles are ˜1.5 seconds each

### B. Cell Lysis

### Example 1. Cell Lysis Using Mechanical and Non-Mechanical Lysis

In this Example, the materials and methods for lysing bacteria (e.g., Staphylococcus aureus) using mechanical lysis (OmniLyse® or centrifugal disk) and non-mechanical lysis (NaOH) are provided.

### Materials

The following materials were used:
1. OmniLyse® Lysis Kit. Available from ClaremontBio.com: http://www.claremontbio.com/OmniLyse_Cell_Lysis_Kits_s/56.htm;
2. 1.7 mL microcentrifuge tubes;
3. mixture of identification (ID) detector probes (100 nM) in 1 M phosphate buffer pH 6.4;
4. 96-well plate containing Luminex MTAG beads functionalized with capture probes;
5. 1×Tm HB=0.1 M Tris pH 8.0, 0.2 M NaCl, 0.08% Triton X-100;
6. 1 M NaOH; and
7. Streptavidin-phycoerythrin conjugate.

### Equipment

The following equipment was used:
1. Shaker Incubator;
2. Biotek 405TS Plate Washer; and
3. Luminex MagPix Assay System.

### Method 1: OmniLyse® and NaOH

The following methodology were used:
1. The OmniLyse® cartridges were pre-wetted by filling the cartridge with filter-sterilized superwater, and emptying with the syringe plunger. This step was repeated one additional time. One OmniLyse® cartridge was needed for each specimen and control.
2. 40 μl of 1 M NaOH was added to 1.7 mL microcentrifuge tubes. 2 extra tubes were included for negative and positive controls.
3. 80 μl of specimen was added to a microcentrifuge tube that contained 40 μl 1 M NaOH and mixed by pipetting.
4. The syringe plunger was used to draw 120 μl of specimen+NaOH from the sample tube into the OmniLyse® cartridge. The OmniLyse® cartridge was turned on for 1 minute.
5. After OmniLyse® treatment, the plunger was used to dispense up to 120 μl of lysate into a tube and incubated at room temperature to complete the 5 minutes of exposure to NaOH.
6. The lysates were neutralized by adding 100 μl of ID detector probe mixture to each tube and mixed by pipetting.
7. 190 μl of neutralized lysate was added to wells in the 96-well ID plate. Negative and positive control lysates were also added.
8. The plate was shaken (without magnet) for 15 minutes on the variable setting with the Biotek plate washer.
9. The beads were washed in the Biotek plate washer using the Biotek Bead Washing Protocol below.
10. While the plate was washing, 2 μl of 1 mg/mL Streptavidin-PE stock was added to 1000 μl 1×Tm HB to yield 2 μg/ml.
11. After the plate was finished washing, 75 μl of 2 μg/mL Streptavidin-PE was added to the appropriate wells.
12. The plate was shaken on variable speed with the Biotek plate washer for 1 minute.
13. The beads were washed with the Biotek plate washer following the protocol listed below.
14. The beads were then measured in the Luminex MagPix instrument.

### Method 2: Centrifugal Disk and NaOH

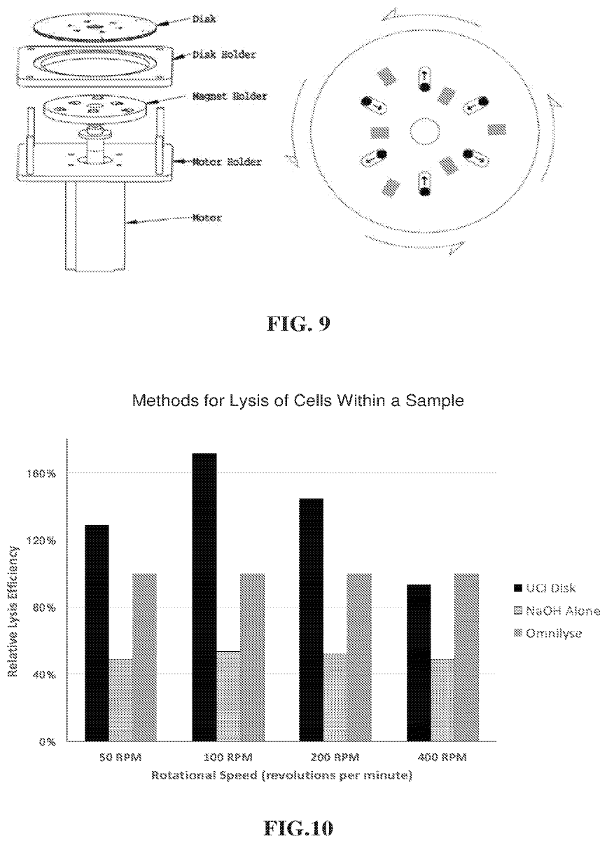

The method for performing mechanical lysis using a centrifugal disk is similar to Method 1 described above, except that the OmniLyse in step 4 of Method 1 was replaced by a centrifugal disk containing a lysis chamber containing zirconium beads and a stainless-steel lysing puck (see FIG. 9). 120 μl of specimen and NaOH from step 3 of Method 1 was placed in the CD lysis chamber and the centrifugal disc was rotated at 100 rpm for 5 minutes. As the centrifugal disc rotated on the spin platform, magnets below the disc caused the stainless-steel lysing pucks to move back and forth in the lysis chamber, which when combined with zirconium beads provided grinding action.

Biotek Bead Washing Protocol (using 96-well plate magnet):
1. Shake on medium for 30 seconds
2. Soak for 30 seconds
3. Aspirate
4. Dispense 200 μl of 1×Tm HB per well
5. Shake on medium for 30 seconds
6. Soak for 30 seconds
7. Aspirate
8. Dispense 200 μl of 1×Tm HB per well
9. Shake on medium for 30 seconds
10. Soak for 30 seconds
11. Aspirate
12. FINAL WASH ONLY: Dispense 50 μl

Biotek 97 well plate washer settings:
1. Aspirate options—Z=43 (5.46 mm above carrier), X=30 (1.37 mm right of center)
2. Dispense options—Z=130 (16.52 mm above carrier), X=0
3. Slow mixing 7 Hz (420 rpm)
4. Medium mixing 13 Hz (780 rpm)
5. Fast mixing was performed at 19 Hz (1140 rpm).

Variable mixing comprised repeated cycles of slow, medium, and fast mixing at approximately 1.5 seconds each.

As shown in FIG. 10, the combination of mechanical lysis and non-mechanical lysis of Staphylococcus areus resulted in more efficient lysis than non-mechanical lysis with NaOH alone. FIG. 10 shows that at 50, 100 and 200 revolutions per minute (RPM), mechanical lysis with a centrifugal disk in combination with non-mechanical lysis using NaOH (first column) and mechanical lysis with OmniLyse® in combination with non-mechanical lysis using NaOH (third column) resulted in more efficient lysis compared to chemical lysis using NaOH alone (second column). The efficacy of the cell lysis was measured by detecting the quantity of rRNA released from identical samples.

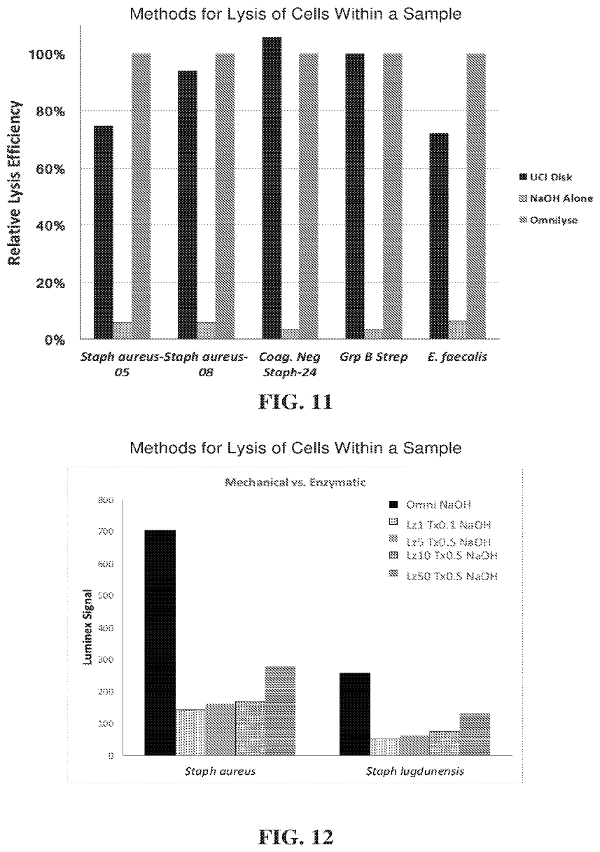

As shown in FIG. 11, mechanical lysis with a centrifugal disk in combination with non-mechanical lysis using NaOH (first column) and mechanical lysis with OmniLyse® in combination with non-mechanical lysis using NaOH (third column) resulted in more efficient lysis for a broad variety of Gram-positive bacteria compared to chemical lysis using NaOH alone (second column). The efficacy of the cell lysis was measured by detecting the quantity of rRNA released from identical samples.

### Example 2. Mechanical Lysis and Non-Mechanical Lysis of Gram-Positive Bacteria Results in More Efficient Detection of rRNA as Compared to a Combination of Enzymatic Lysis, Detergent Lysis and Chemical Lysis

In this Example, using the relevant materials and methodology described in Example 1, Gram-positive bacteria were lysed using a two-step lysis using either (a) Step 1: enzymatic lysis and detergent lysis, and Step 2: chemical lysis (e.g., Step 1: Triton X-100 and lysozyme, and Step 2: NaOH); or (b) Step 1: mechanical lysis and Step 2: chemical lysis (e.g., Step 1: OmniLyse® and Step 2: NaOH), followed by detection of rRNA using a Luminex® instrument.

As shown in FIG. 12, the detection of rRNA was greatly increased following mechanical lysis using OmniLyse® in combination with chemical lysis using NaOH (first column) as compared to the detection of rRNA following enzymatic lysis using lysozyme and detergent lysis using Triton X-100 in combination with chemical lysis using NaOH.

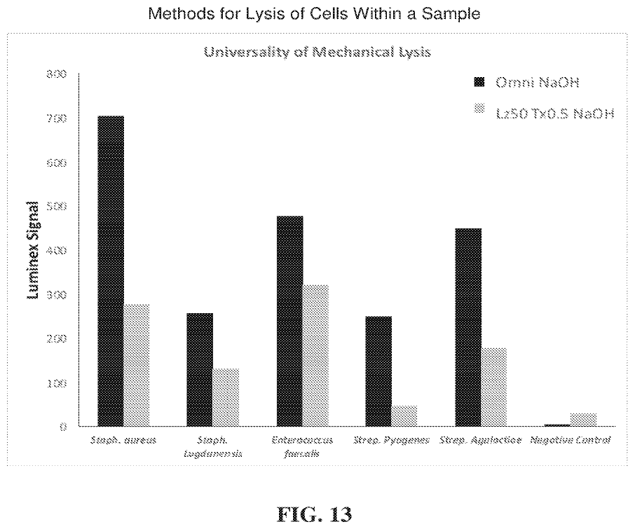

As shown in FIG. 13, mechanical lysis using OmniLyse® in combination with chemical lysis using NaOH (first column) resulted in improved detection of rRNA from a broad variety of Gram-positive bacteria (e.g., Staphylococcus aureus, Staphylococcus lugdunensis, Enterococcus faecalis, Streptococcus pyogenes, and Streptococcus Agalactiae) compared to enzymatic lysis using lysozyme and detergent lysis using Triton X-100 in combination with chemical lysis using NaOH.

These results demonstrate that the first step of enzyme plus detergent followed by NaOH treatment results in less efficient detection of rRNA from Gram-positive cells than the combination of mechanical lysis plus NaOH.

### Example 3. Impact of the Duration of Mechanical Lysis and Concentration of NaOH on rRNA Detection

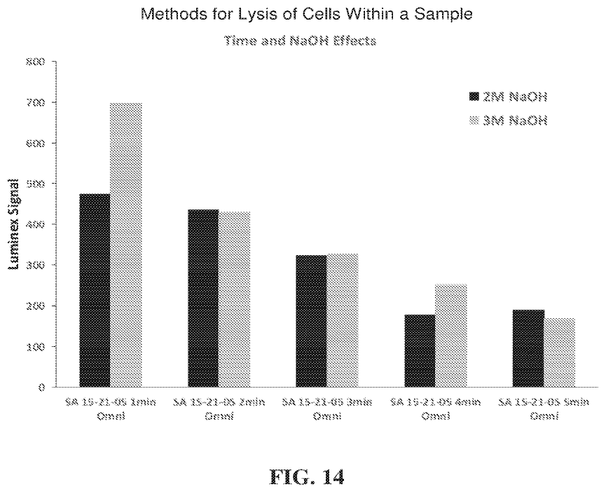

In this Example, using the relevant materials and methodology described in Example 1, the impact of the duration of mechanical lysis and concentration of NaOH on rRNA detection from Staphylococcus aureus was investigated. In the first step, bacteria were lysed for 1, 2, 3, 4, or 5 minutes using OmniLyse® and then chemically lysed using 2 M NaOH or 3 M NaOH for a duration of 5 minutes. As shown in FIG. 14, an optimal signal was achieved with mechanical lysis for 1 minute followed by chemical lysis using 3 M NaOH.

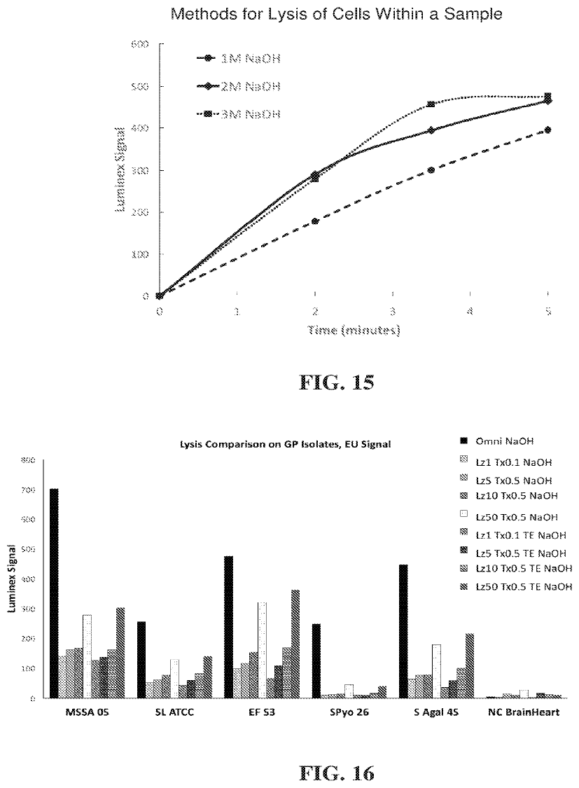

A separate experiment was performed to determine the optimal duration of NaOH treatment following a 1-minute mechanical lysis (OmniLyse®). For all NaOH concentrations, the optimal duration of NaOH treatment was found to be 5 minutes (FIG. 15).

### Example 4. Efficacy of Various Concentrations of Lysozyme Lysis Buffer on Gram-Positive Isolates

In step one of this example, the impact of biological (enzymatic in this case) lysis at different concentrations was investigated and compared to a combination of mechanical and alkaline lysis. During this experiment, a series of Gram-positive bacteria were lysed using different concentrations of lysozyme enzyme solution, either with or without the addition of 1-minute mechanical lysis (OmniLyse®). Following lysis, the cell lysate was contacted with specific capture probes and detector probes, using the relevant materials and methodology described in Example 1, to detect one or more nucleotide sequences in the cell lysate.

In step two, a separate experiment was performed, using the relevant materials and methodology described in Example 1, where Gram-positive bacteria were subjected to NaOH treatment following 1-minute mechanical lysis (OmniLyse®). The results for step one and step two were compared as shown in FIG. 16.

### Experimental Materials

The following materials were used:
1. OmniLyse® Lysis Kit. Available from ClaremontBio.com: http://www.claremontbio.com/OmniLyse_Cell_Lysis_Kits_s/56.htm;
2. Bacteria samples including: MSSA 15-21-05; Staph Lugdunensis ATCC; E. faecalis 07-09-53; Strep. pyogenes 15-21-26; and Strep. agalactiae 07-09-45
3. Lysis buffer including:
   (a) Lysozyme @ 1 mg/mL, Triton X-100 @ 0.1%, in H₂O
   (b) Lysozyme @ 5 mg/mL, Triton X-100 @ 0.5%, in H₂O
   (c) Lysozyme @ 10 mg/mL, Triton X-100 @ 0.5%, in H₂O
   (d) Lysozyme @ 50 mg/mL, Triton X-100 @ 0.5%, in H₂O
   (e) Lysozyme @ 1 mg/mL, Triton X-100 @ 0.1%, in 20 mM Tris-HCl 2 mM EDTA pH 8.0
   (f) Lysozyme @ 5 mg/mL, Triton X-100 @ 0.5%, in 20 mM Tris-HCl 2 mM EDTA pH 8.0
   (g) Lysozyme @ 10 mg/mL, Triton X-100 @ 0.5%, in 20 mM Tris-HCl 2 mM EDTA pH 8.0
   (h) Lysozyme @ 50 mg/mL, Triton X-100 @ 0.5%, in 20 mM Tris-HCl 2 mM EDTA pH 8.0
4. 96-well plate containing Luminex MTAG beads functionalized with capture probes; and
5. 1 M NaOH.

### Experimental Methods

The following experimental variables were used for the Lysozyme Buffer Set-Up:
The Lysozyme Buffers were made the same for every concentration, including:
   a. 40 uL Bacteria+10 uL Enzymatic Lysis Buffer (5 min @ room temperature)
   b. 25 uL 1 M NaOH (5 min)
   c. 75 uL 1 M Phosphate Buffer

### Results

As shown in FIG. 8, the best enzymatic lysis condition used 50 mg/mL Lysozyme and 0.5% Triton X-100—i.e., 3(d) and 3(h) above.

### Example 5. Testing Relationship Between Strength of NaOH and Timing of OmniLyse®

### Experimental Methods

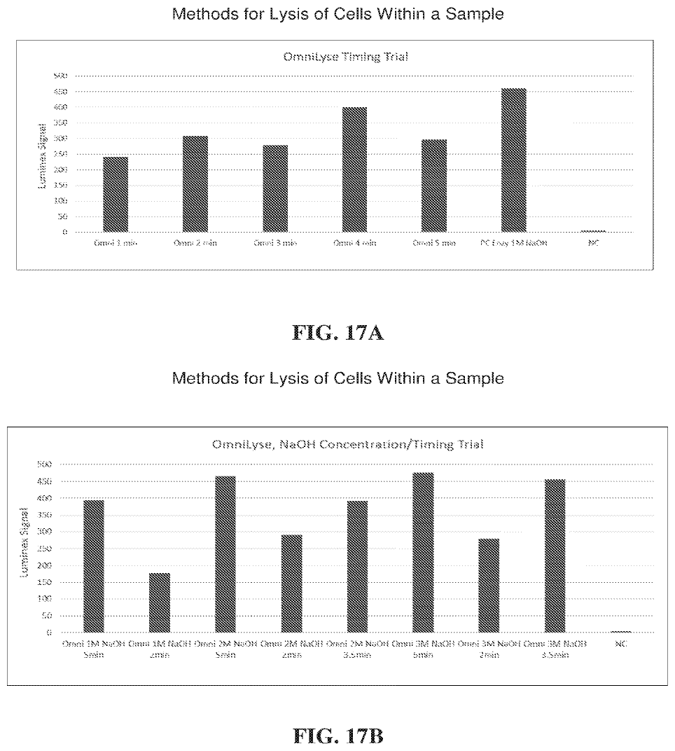

In this example, two experiments were performed. In the first experiment, using the relevant materials and methodology described in Example 1, the relationship between strength of NaOH and timing of Omnilyse® was investigated. In the first step, samples of Gram-positive bacteria (Staphylococcus aureus) were lysed for 1, 2, 3, 4, or 5 minutes using OmniLyse® and then chemically lysed using 1 M NaOH for 5 minutes after OmniLyse® treatment. Results from this lysis were compared to enzymatic lysis as a control (See FIG. 17A)

In a second experiment, bacteria lysis of Gram-positive bacteria (Staphylococcus aureus) was performed with OmniLyse® for 2, 3.5 or 5 minutes with 1M, 2 M or 3 M NaOH (See FIG. 17B).

### Results

As shown in FIGS. 17A and 17B, the combination of mechanical and non-mechanical lysis has proven to be effective in lysis of Gram-positive bacteria. The highest signal was found using 3 M NaOH for 5 minutes, 3 M for 3.5 minutes and 2 M for 5 minutes.

### Example 6. Testing Combination Lysis Methods on Eukaryotic Fungal Cells (Candida Albicans)

In this example, using the relevant materials and methodology described in Example 1, the effectiveness of different lysis methods was tested on different cell types, including Gram-negative cells, Gram-positive cells and eukaryotic fungal cells.

### Experimental Materials

1. The following bacterial samples were used:
   a. 10 Gram-negative, including E. coli, P. mirabilis, K. pneumoniae, K oxytoca, E. hormaechei, E. aerogenes, E. cloacae, P. aeruginosa, C. freundii, and S. marcescens
   b. 9 Gram-positive organisms, including S. aureus, S. lugdunensis, E. faecalis, E. faecium, S. agalactiae, S. pneumoniae, S. viridans, and S. pyogenes
   c. 1 yeast, C. albicans
2. All bacteria were grown in MH2+5% LAKED horse blood+1 ug/mL RnaseA
3. C. albicans was grown in RPMI overnight

### Experimental Methods

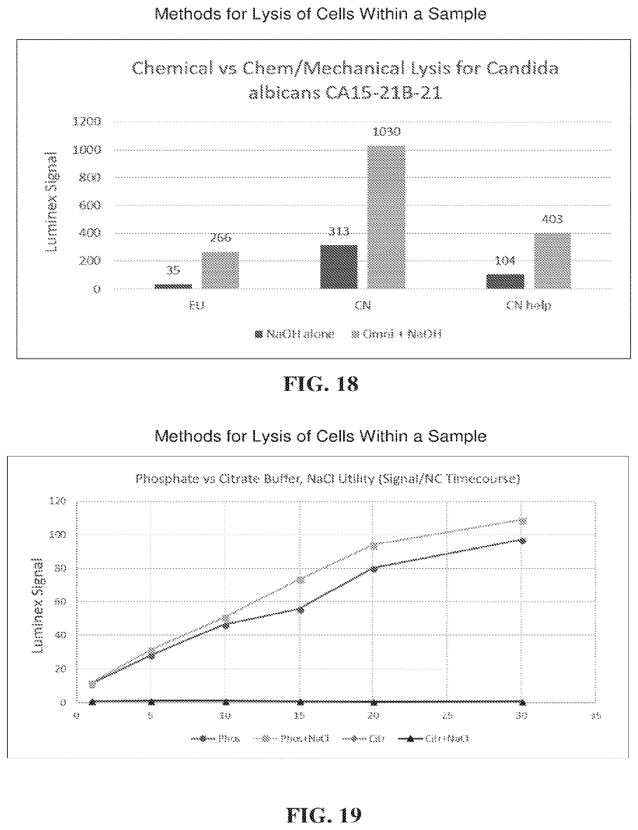

For Gram-negative cells, alkaline lysis alone was used. For Gram-positive cells, a combination of alkaline lysis with OmniLyse® mechanical lysis was used. For eukaryotic fungal cells both alkaline lysis alone and a combination of alkaline lysis with OmniLyse® mechanical lysis were tested and compared. When the combination was used, alkaline (chemical) lysis with 1 M NaOH was performed for 5 minutes and Omnilyse® (mechanical) was performed for the first 2 minutes of the 5 minutes alkaline (1 M NaOH) lysis. Results for probe specificity following the lysis of each cell type are shown in FIG. 18.

### Results

As shown in FIG. 10, higher signals were obtained with the combination of chemical and mechanical lysis as detected with eumicrobial (EU) or candida (CN or CN-Help) probes.

### Example 7. Comparison of Buffers for Neutralizing Lysate

### Experimental Methods

In this experiment, cell lysate samples were neutralized by contacting the samples with a buffer solution. During this experiment a series of different buffers were used, including: 1M Phosphate buffer (PB); 1 M PB+1 M NaCl; 1 M Citrate buffer (CB); and 1 M CB+1 M NaCl and their ability to neutralize NaOH in the lysate was compared. See FIG. 11.

### Results

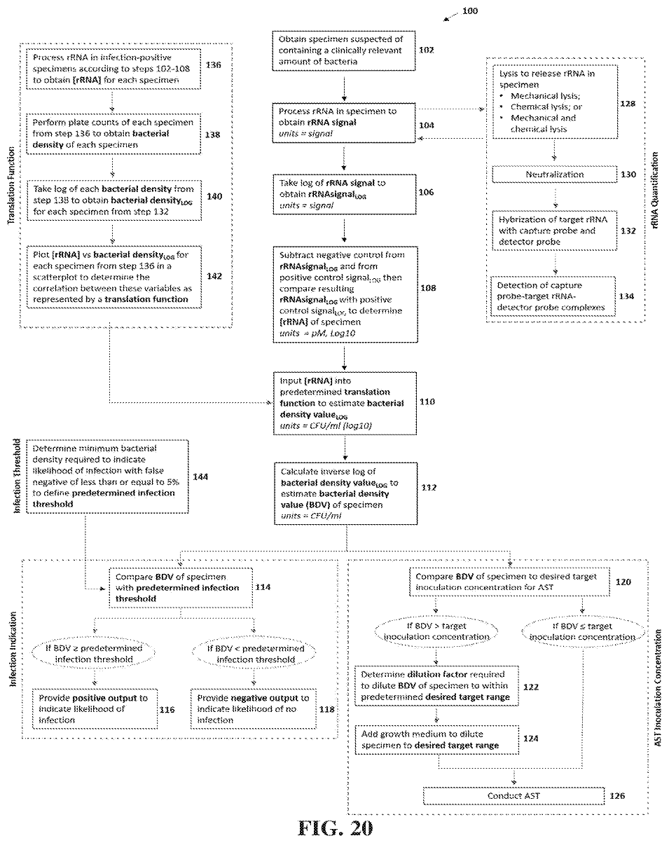

As shown in FIG. 19, when compared to an equal molarity strength of Citrate buffer, the phosphate buffer was much better at neutralizing the lysate.

### Antimicrobial Agents

The experiments discussed above have determined the following predetermined concentrations, when the desired inoculation period is 90-120 minutes across a wide range of gram negative pathogens:

### Gentamicin

In some embodiments, the at least one antimicrobial agent includes gentamicin. In some embodiments, the predetermined concentration of gentamicin is equal to the CLSI MIC susceptible breakpoint. In some embodiments, the predetermined concentration of gentamicin is less than the CLSI MIC susceptible breakpoint.

In some embodiments, the predetermined concentration of gentamicin is at least 2 μg/mL. In some embodiments, the predetermined concentration of gentamicin is at least 4 μg/mL. In some embodiments, the predetermined concentration of gentamicin is 2 μg/mL. In some embodiments, the predetermined concentration of gentamicin is 4 μg/mL.

### Ciprofloxacin

In some embodiments, the at least one antimicrobial agent includes ciprofloxacin. In some embodiments, the predetermined concentration of ciprofloxacin is greater than the CLSI MIC susceptible breakpoint. In some embodiments, the supratherapeutic concentration of ciprofloxacin is greater than the CLSI MIC intermediate breakpoint. In some embodiments, the predetermined concentration of ciprofloxacin is equal to the CLSI MIC resistant breakpoint.

In some embodiments, the predetermined concentration of ciprofloxacin is at least 4 μg/mL. In some embodiments, the predetermined concentration of ciprofloxacin is 4 μg/mL.

### Cefazolin

In some embodiments, the at least one antimicrobial agent includes cefazolin. In some embodiments, the predetermined concentration of cefazolin is greater than the CLSI MIC susceptible breakpoint. In some embodiments, the predetermined concentration of cefazolin is greater than the CLSI MIC intermediate breakpoint. In some embodiments, the predetermined concentration of cefazolin is greater than the CLSI MIC resistant breakpoint.

In some embodiments, the predetermined concentration of cefazolin is greater than 40 μg/mL. In some embodiments, the predetermined concentration of cefazolin is greater than 50 μg/mL. In some embodiments, the predetermined concentration of cefazolin is at least 64 μg/mL. In some embodiments, the predetermined concentration of cefazolin is 64 μg/mL.

### Ceftriaxone

In some embodiments, the at least one antimicrobial agent includes ceftriaxone. In some embodiments, the predetermined concentration of ceftriaxone is greater than the CLSI MIC susceptible breakpoint. In some embodiments, the predetermined concentration of ceftriaxone is greater than the CLSI MIC intermediate breakpoint. In some embodiments, the predetermined concentration of ceftriaxone is greater than the CLSI MIC resistant breakpoint.

In some embodiments, the predetermined concentration of ceftriaxone is greater than 20 μg/mL. In some embodiments, the predetermined concentration of ceftriaxone is greater than 25 μg/mL. In some embodiments, the predetermined concentration of ceftriaxone is at least 32 μg/mL. In some embodiments, the predetermined concentration of ceftriaxone is 32 μg/mL.

### Cefepime

In some embodiments, the at least one antimicrobial agent includes cefepime. In some embodiments, the predetermined concentration of cefepime is greater than the CLSI MIC susceptible breakpoint. In some embodiments, the predetermined concentration of cefepime is greater than the CLSI MIC intermediate breakpoint. In some embodiments, the supratherapeutic concentration of cefepime is greater than the CLSI MIC resistant breakpoint.

In some embodiments, the predetermined concentration of cefepime is greater than 30 μg/mL. In some embodiments, the predetermined concentration of cefepime is at least 32 μg/mL. In some embodiments, the predetermined concentration of cefepime is greater than 40 μg/mL. In some embodiments, the predetermined concentration of cefepime is greater than 50 μg/mL. In some embodiments, the predetermined concentration of cefepime is at least 64 μg/mL. In some embodiments, the predetermined concentration of cefepime is 32 μg/mL. In some embodiments, the predetermined concentration of cefepime is 64 μg/mL.

### Ampicillin

In some embodiments, the at least one antimicrobial agent includes ampicillin. In some embodiments, the predetermined concentration of ampicillin is greater than the CLSI MIC susceptible breakpoint. In some embodiments, the predetermined concentration of ampicillin is greater than the CLSI MIC intermediate breakpoint. In some embodiments, the predetermined concentration of ampicillin is greater than the CLSI MIC resistant breakpoint.

In some embodiments, the predetermined concentration of ampicillin is greater than 50 μg/mL. In some embodiments, the predetermined concentration of ampicillin is greater than 100 μg/mL. In some embodiments, the predetermined concentration of ampicillin is at least 128 μg/mL. In some embodiments, the predetermined concentration of ampicillin is 128 μg/mL.

### Imipenem

In some embodiments, the at least one antimicrobial agent includes imipenem. In some embodiments, the predetermined concentration of imipenem is greater than the CLSI MIC susceptible breakpoint. In some embodiments, the predetermined concentration of imipenem is greater than the CLSI MIC intermediate breakpoint. In some embodiments, the predetermined concentration of imipenem is between the CLSI MIC intermediate and resistant breakpoints. In some embodiments, the predetermined concentration of imipenem is greater than the CLSI MIC resistant breakpoint.

In some embodiments, the predetermined concentration of imipenem is greater than 2 μg/mL. In some embodiments, the predetermined concentration of imipenem is greater than 3 μg/mL. In some embodiments, the predetermined concentration of imipenem is at least 4 μg/mL.

### Trimethoprim

In some embodiments, the at least one antimicrobial agent includes trimethoprim. In some embodiments, the predetermined concentration of trimethoprim is greater than the CLSI MIC susceptible breakpoint. In some embodiments, the predetermined concentration of trimethoprim is greater than the CLSI MIC intermediate breakpoint. In some embodiments, the predetermined concentration of trimethoprim is equal to the resistant breakpoint. In some embodiments, the predetermined concentration of trimethoprim is greater than the CLSI MIC resistant breakpoint.

In some embodiments, the predetermined concentration of trimethoprim is at least 4 μg/mL. In some embodiments, the predetermined concentration of trimethoprim is 4 μg/mL.

### Sulfamethoxazole

In some embodiments, the at least one antimicrobial agent includes sulfamethoxazole. In some embodiments, the predetermined concentration of sulfamethoxazole is greater than the CLSI MIC susceptible breakpoint. In some embodiments, the predetermined concentration of sulfamethoxazole is greater than the CLSI MIC intermediate breakpoint. In some embodiments, the predetermined concentration of sulfamethoxazole is equal to the CLSI MIC resistant breakpoint. In some embodiments, the predetermined concentration of sulfamethoxazole is greater than the CLSI MIC resistant breakpoint.

In some embodiments, the predetermined concentration of sulfamethoxazole is at least 76 μg/mL. In some embodiments, the predetermined concentration of sulfamethoxazole is 76 μg/mL

### Amikacin

In some embodiments, the at least one antimicrobial agent includes amikacin. In some embodiments, the predetermined concentration of ampicillin is less than the CLSI MIC susceptible breakpoint. In some embodiments, the predetermined concentration of ampicillin is equal to the CLSI MIC susceptible breakpoint. In some embodiments, the predetermined concentration of ampicillin is greater than the CLSI MIC susceptible breakpoint. In some embodiments, the predetermined concentration of ampicillin is equal to the CLSI MIC intermediate breakpoint.

In some embodiments, the predetermined concentration of amikacin is at least 8 μg/mL. In some embodiments, the predetermined concentration of amikacin is at least 16 μg/mL. In some embodiments, the predetermined concentration of amikacin is at least 32 μg/mL. In some embodiments, the predetermined concentration of amikacin is 8 μg/mL. In some embodiments, the predetermined concentration of amikacin is 16 μg/mL. In some embodiments, the predetermined concentration of amikacin is 32 μg/mL.

### Nitrofurantoin

In some embodiments, wherein the at least one antimicrobial agent includes nitrofurantoin. In some embodiments, the predetermined concentration of nitrofurantoin is less than the CLSI MIC susceptible breakpoint.

In some embodiments, the predetermined concentration of nitrofurantoin is at least 16 μg/mL. In some embodiments, the predetermined concentration of nitrofurantoin is 6 μg/mL.

### Fosfomycin

In some embodiments, wherein the at least one antimicrobial agent includes fosfomycin. In some embodiments, the predetermined concentration of fosfomycin is less than the CLSI MIC susceptible breakpoint. In some embodiments, the predetermined concentration of fosfomycin is equal to the CLSI MIC susceptible breakpoint.

In some embodiments, the predetermined concentration of fosfomycin is at least 64 μg/mL. In some embodiments, the predetermined concentration of fosfomycin is 64 μg/mL.

### Piperacillin

In some embodiments, the at least one antimicrobial agent includes piperacillin. In some embodiments, the predetermined concentration of piperacillin is greater than the CLSI MIC susceptible breakpoint. In some embodiments, the predetermined concentration of piperacillin is greater than the CLSI MIC intermediate breakpoint. In some embodiments, the predetermined concentration of piperacillin is between the CLSI MIC intermediate and resistant breakpoints. In some embodiments, the predetermined concentration of piperacillin is greater than the CLSI MIC resistant breakpoint.

In some embodiments, the predetermined concentration of piperacillin is greater than 10 μg/mL. In some embodiments, the predetermined concentration of piperacillin is greater than 12 μg/mL. In some embodiments, the predetermined concentration of piperacillin is at least 16 μg/mL.

### Tazobactam

In some embodiments, the at least one antimicrobial agent includes tazobactam. In some embodiments, the predetermined concentration of tazobactam is greater than the CLSI MIC susceptible breakpoint. In some embodiments, the predetermined concentration of tazobactam is greater than the CLSI MIC intermediate breakpoint. In some embodiments, the predetermined concentration of tazobactam is between the CLSI MIC intermediate and resistant breakpoints. In some embodiments, the predetermined concentration of tazobactam is greater than the CLSI MIC resistant breakpoint.

In some embodiments, the predetermined concentration of tazobactam is greater than 2 μg/mL. In some embodiments, the predetermined concentration of tazobactam is greater than 3 μg/mL. In some embodiments, the predetermined concentration of tazobactam is at least 4 μg/mL. In some configurations, Tazobactam can be used in combination with piperacillin in a combination antibiotic dosage for an AST test. Surprisingly, it was discovered that when varying the concentration of the combination dosage that acceptable results were found when the concentration of Tazobactam was held constant and the dosages of piperacillin were varied in the following ratios 128/4, 64/4 and 32/4 (piperacillin/tazobactam concentration in 3 ug/mL).

### Amoxicillin

In some embodiments, the at least one antimicrobial agent includes amoxicillin. In some embodiments, the predetermined concentration of amoxicillin is greater than the CLSI MIC susceptible breakpoint. In some embodiments, the predetermined concentration of amoxicillin is equal to the CLSI MIC intermediate breakpoint. In some embodiments, the predetermined concentration of amoxicillin is greater than the CLSI MIC intermediate breakpoint. In some embodiments, the predetermined concentration of amoxicillin is equal to the CLSI MIC resistant breakpoint. In some embodiments, the predetermined concentration of amoxicillin is greater than the CLSI MIC resistant breakpoint.

In some embodiments, the predetermined concentration of amoxicillin is greater than 16 μg/mL. In some embodiments, the predetermined concentration of amoxicillin is greater than 32 μg/mL. In some embodiments, the predetermined concentration of amoxicillin is at least 16 μg/mL. In some embodiments, the predetermined concentration of amoxicillin is at least 32 μg/mL. In some embodiments, the predetermined concentration of amoxicillin is at least 64 μg/mL. In some embodiments, the predetermined concentration of amoxicillin is 16 μg/mL. In some embodiments, the predetermined concentration of amoxicillin is 32 μg/mL. In some embodiments, the predetermined concentration of amoxicillin is 64 μg/mL.

### Clavulanate

In some embodiments, the at least one antimicrobial agent includes clavulanate. In some embodiments, the predetermined concentration of clavulanate is greater than the CLSI MIC susceptible breakpoint. In some embodiments, the predetermined concentration of clavulanate is equal to the CLSI MIC intermediate breakpoint. In some embodiments, the predetermined concentration of clavulanate is greater than the CLSI MIC intermediate breakpoint. In some embodiments, the predetermined concentration of clavulanate is equal to the CLSI MIC resistant breakpoint. In some embodiments, the predetermined concentration of clavulanate is greater than the CLSI MIC resistant breakpoint.

In some embodiments, the predetermined concentration of clavulanate is greater than 8 μg/mL. In some embodiments, the predetermined concentration of clavulanate is greater than 16 μg/mL. In some embodiments, the predetermined concentration of clavulanate is at least 8 μg/mL. In some embodiments, the predetermined concentration of clavulanate is at least 16 μg/mL. In some embodiments, the predetermined concentration of clavulanate is at least 32 μg/mL. In some embodiments, the predetermined concentration of clavulanate is 8 μg/mL. In some embodiments, the predetermined concentration of clavulanate is 16 μg/mL. In some embodiments, the predetermined concentration of clavulanate is 32 μg/mL.

### Ertapenem

In some embodiments, the at least one antimicrobial agent includes ertapenem. In some embodiments, the predetermined concentration of ertapenem is greater than the CLSI MIC susceptible breakpoint. In some embodiments, the predetermined concentration of ertapenem is equal to the CLSI MIC intermediate breakpoint. In some embodiments, the predetermined concentration of ertapenem is greater than the CLSI MIC intermediate breakpoint. In some embodiments, the predetermined concentration of ertapenem is equal to the CLSI MIC resistant breakpoint.

In some embodiments, the predetermined concentration of ertapenem is greater than 2 μg/mL. In some embodiments, the predetermined concentration of ertapenem is greater than 4 μg/mL. In some embodiments, the predetermined concentration of ertapenem is at least 2 μg/mL. In some embodiments, the predetermined concentration of ertapenem is at least 4 μg/mL. In some embodiments, the predetermined concentration of ertapenem is 2 μg/mL. In some embodiments, the predetermined concentration of ertapenem is 4 μg/mL.

### Meropenem

In some embodiments, the at least one antimicrobial agent includes meropenem. In some embodiments, the predetermined concentration of meropenem is greater than the CLSI MIC susceptible breakpoint. In some embodiments, the predetermined concentration of meropenem is equal to the CLSI MIC intermediate breakpoint. In some embodiments, the predetermined concentration of meropenem is greater than the CLSI MIC intermediate breakpoint. In some embodiments, the predetermined concentration of meropenem is equal to the CLSI MIC resistant breakpoint.

In some embodiments, the predetermined concentration of meropenem is greater than 2 μg/mL. In some embodiments, the predetermined concentration of meropenem is greater than 4 μg/mL. In some embodiments, the predetermined concentration of meropenem is at least 2 μg/mL. In some embodiments, the predetermined concentration of meropenem is at least 4 μg/mL. In some embodiments, the predetermined concentration of meropenem is 2 μg/mL. In some embodiments, the predetermined concentration of meropenem is 4 μg/mL.

In some embodiments, a microorganism is exposed to two or more antimicrobial agents simultaneously. For instance, a culture media of an inoculate may comprise two or more antimicrobial agents. In some embodiments, a culture may comprise a beta-lactam antibiotic and a beta-lactamase inhibitor (BLI). In some embodiments, a culture media comprises two or more antimicrobial agents, wherein the two or more antimicrobial agents are selected from the group of gentamicin, ciprofloxacin, cefazolin, ceftriaxone, cefepime, ampicillin, trimethoprim, sulfamethoxazole, amikacin, nitrofurantoin, fosfomycin, amoxicillin, clavulanate, ertapenem, and meropenem. In some embodiments, a culture media comprises trimethoprim and sulfamethoxazole. In some embodiments, a culture media comprises amoxicillin and clavulanate.

For some antimicrobial agents, and for some purposes, the predetermined concentration of the antimicrobial agent is at least 5%, 10%, 15%, 20%, 25%, 30%, 35%, 40%, 45%, 50%, 55%, 60%, 65%, 70%, 75%, 80%, 85%, 90%, 95% or 100% or greater than the therapeutic concentration of the antimicrobial agent. In some embodiments, the predetermined concentration of the antimicrobial agent is at least 20% or greater than the therapeutic concentration of the antimicrobial agent. In some embodiments, the predetermined concentration of the antimicrobial agent is at least 40% or greater than the therapeutic concentration of the antimicrobial agent. In some embodiments, the predetermined concentration of the antimicrobial agent is at least 50% or greater than the therapeutic concentration of the antimicrobial agent. In some embodiments, the predetermined concentration of the antimicrobial agent is at least 70% or greater than the therapeutic concentration of the antimicrobial agent. In some embodiments, the predetermined concentration of the antimicrobial agent is at least 80% or greater than the therapeutic concentration of the antimicrobial agent. In some embodiments, a predetermined concentration of the antimicrobial agent is equal to the therapeutic concentration of the antimicrobial agent. In some embodiments, the predetermined enhanced-rate concentration of the antimicrobial agent is less than the therapeutic concentration of the antimicrobial agent.

In some embodiments, the predetermined concentration of an antimicrobial agent is at least 1.5, 2, 2.5, 3, 3.5, 4, 4.5, 5, 5.5, 6, 6.5, 7, 7.5, 8, 8.5, 9, 9.5, or 10-fold or greater than the therapeutic concentration of the antimicrobial agent. In some embodiments, the predetermined enhanced-rate concentration of the antimicrobial agent is at least 1.5-fold or greater than the therapeutic concentration of the antimicrobial agent. In some embodiments, the predetermined enhanced-rate concentration of the antimicrobial agent is at least 2-fold or greater than the predetermined enhanced-rate concentration of the antimicrobial agent. In some embodiments, the predetermined enhanced-rate concentration of the antimicrobial agent is at least 3-fold or greater than the therapeutic concentration of the antimicrobial agent. In some embodiments, the predetermined enhanced-rate concentration of the antimicrobial agent is at least 4-fold or greater than the therapeutic concentration of the antimicrobial agent. In some embodiments, the predetermined enhanced-rate concentration of the antimicrobial agent is at least 5-fold or greater than the therapeutic concentration of the antimicrobial agent.

In some embodiments, a predetermined concentration of an antimicrobial agent is at least 5%, 10%, 15%, 20%, 25%, 30%, 35%, 40%, 45%, 50%, 55%, 60%, 65%, 70%, 75%, 80%, 85%, 90%, 95% or 100% or greater than the susceptible CLSI MIC breakpoint of the antimicrobial agent. In some embodiments, a predetermined enhanced-rate concentration of the antimicrobial agent is at least 20% or greater than the susceptible CLSI MIC breakpoint of the antimicrobial agent. In some embodiments, a predetermined enhanced-rate concentration of the antimicrobial agent is at least 40% or greater than the susceptible CLSI MIC breakpoint of the antimicrobial agent. In some embodiments, a predetermined enhanced-rate concentration of the antimicrobial agent is at least 50% or greater than the susceptible CLSI MIC breakpoint of the antimicrobial agent. In some embodiments, a predetermined enhanced-rate concentration of the antimicrobial agent is at least 70% or greater than the susceptible CLSI MIC breakpoint of the antimicrobial agent. In some embodiments, a predetermined enhanced-rate concentration of the antimicrobial agent is at least 80% or greater than the susceptible CLSI MIC breakpoint of the antimicrobial agent. In some embodiments, the predetermined concentration of an antimicrobial agent is equal to the susceptible CLSI MIC breakpoint. In some embodiments, the predetermined concentration of an antimicrobial agent is less than the susceptible CLSI MIC breakpoint.

In some embodiments, the supratherapeutic concentration of an antimicrobial agent is at least 1.5, 2, 2.5, 3, 3.5, 4, 4.5, 5, 5.5, 6, 6.5, 7, 7.5, 8, 8.5, 9, 9.5, or 10-fold or greater than the susceptible CLSI MIC breakpoint of the antimicrobial agent. In some embodiments, the predetermined enhanced-rate concentration of the antimicrobial agent is at least 1.5-fold or greater than the susceptible CLSI MIC breakpoint of the antimicrobial agent. In some embodiments, the predetermined enhanced-rate concentration of the antimicrobial agent is at least 2-fold or greater than the susceptible CLSI MIC breakpoint of the antimicrobial agent. In some embodiments, the predetermined enhanced-rate concentration of the antimicrobial agent is at least 3-fold or greater than the susceptible CLSI MIC breakpoint of the antimicrobial agent. In some embodiments, the predetermined enhanced-rate concentration of the antimicrobial agent is at least 4-fold or greater than the susceptible CLSI MIC breakpoint of the antimicrobial agent. In some embodiments, the predetermined enhanced-rate concentration of the antimicrobial agent is at least 5-fold or greater than the susceptible CLSI MIC breakpoint of the antimicrobial agent.

In some embodiments, a predetermined concentration of an antimicrobial agent is at least 5%, 10%, 15%, 20%, 25%, 30%, 35%, 40%, 45%, 50%, 55%, 60%, 65%, 70%, 75%, 80%, 85%, 90%, 95%, or 100% or greater than the intermediate CLSI MIC breakpoint of the antimicrobial agent. In some embodiments, a predetermined enhanced-rate concentration of the antimicrobial agent is at least 20% or greater than the intermediate CLSI MIC breakpoint of the antimicrobial agent. In some embodiments, a predetermined enhanced-rate concentration of the antimicrobial agent is at least 40% or greater than the intermediate CLSI MIC breakpoint of the antimicrobial agent. In some embodiments, a predetermined enhanced-rate concentration of the antimicrobial agent is at least 50% or greater than the intermediate CLSI MIC breakpoint of the antimicrobial agent. In some embodiments, a predetermined enhanced-rate concentration of the antimicrobial agent is at least 70% or greater than the intermediate CLSI MIC breakpoint of the antimicrobial agent. In some embodiments, a predetermined enhanced-rate concentration of the antimicrobial agent is at least 80% or greater than the intermediate CLSI MIC breakpoint of the antimicrobial agent. In some embodiments, the predetermined concentration of an antimicrobial agent is equal to the intermediate CLSI MIC breakpoint. In some embodiments, the predetermined concentration of an antimicrobial agent is less than the intermediate CLSI MIC breakpoint.

In some embodiments, the supratherapeutic concentration of an antimicrobial agent is at least 1.5, 2, 2.5, 3, 3.5, 4, 4.5, 5, 5.5, 6, 6.5, 7, 7.5, 8, 8.5, 9, 9.5, or 10-fold or greater than the intermediate CLSI MIC breakpoint of the antimicrobial agent. In some embodiments, the predetermined enhanced-rate concentration of the antimicrobial agent is at least 1.5-fold or greater than the intermediate CLSI MIC breakpoint of the antimicrobial agent. In some embodiments, the predetermined enhanced-rate concentration of the antimicrobial agent is at least 2-fold or greater than the intermediate CLSI MIC breakpoint of the antimicrobial agent. In some embodiments, the predetermined enhanced-rate concentration of the antimicrobial agent is at least 3-fold or greater than the intermediate CLSI MIC breakpoint of the antimicrobial agent. In some embodiments, the predetermined enhanced-rate concentration of the antimicrobial agent is at least 4-fold or greater than the intermediate CLSI MIC breakpoint of the antimicrobial agent. In some embodiments, the predetermined enhanced-rate concentration of the antimicrobial agent is at least 5-fold or greater than the intermediate CLSI MIC breakpoint of the antimicrobial agent.

In some embodiments, a predetermined concentration of an antimicrobial agent is at least 5%, 10%, 15%, 20%, 25%, 30%, 35%, 40%, 45%, 50%, 55%, 60%, 65%, 70%, 75%, 80%, 85%, 90%, 95%, or 100% or greater than the resistant CLSI MIC breakpoint of the antimicrobial agent. In some embodiments, a predetermined enhanced-rate concentration of the antimicrobial agent is at least 20% or greater than the resistant CLSI MIC breakpoint of the antimicrobial agent. In some embodiments, a predetermined enhanced-rate concentration of the antimicrobial agent is at least 40% or greater than the resistant CLSI MIC breakpoint of the antimicrobial agent. In some embodiments, a predetermined enhanced-rate concentration of the antimicrobial agent is at least 50% or greater than the resistant CLSI MIC breakpoint of the antimicrobial agent. In some embodiments, a predetermined enhanced-rate concentration of the antimicrobial agent is at least 70% or greater than the resistant CLSI MIC breakpoint of the antimicrobial agent. In some embodiments, a predetermined enhanced-rate concentration of the antimicrobial agent is at least 80% or greater than the resistant CLSI MIC breakpoint of the antimicrobial agent. In some embodiments, the predetermined concentration of an antimicrobial agent is equal to the resistant CLSI MIC breakpoint. In some embodiments, the predetermined concentration of an antimicrobial agent is less than the resistant CLSI MIC breakpoint.

In some embodiments, the supratherapeutic concentration of an antimicrobial agent is at least 1.5, 2, 2.5, 3, 3.5, 4, 4.5, 5, 5.5, 6, 6.5, 7, 7.5, 8, 8.5, 9, 9.5, or 10-fold or greater than the resistant CLSI MIC breakpoint of the antimicrobial agent. In some embodiments, the predetermined enhanced-rate concentration of the antimicrobial agent is at least 1.5-fold or greater than the resistant CLSI MIC breakpoint of the antimicrobial agent. In some embodiments, the predetermined enhanced-rate concentration of the antimicrobial agent is at least 2-fold or greater than the resistant CLSI MIC breakpoint of the antimicrobial agent. In some embodiments, the predetermined enhanced-rate concentration of the antimicrobial agent is at least 3-fold or greater than the resistant CLSI MIC breakpoint of the antimicrobial agent. In some embodiments, the predetermined enhanced-rate concentration of the antimicrobial agent is at least 4-fold or greater than the resistant CLSI MIC breakpoint of the antimicrobial agent. In some embodiments, the predetermined enhanced-rate concentration of the antimicrobial agent is at least 5-fold or greater than the resistant CLSI MIC breakpoint of the antimicrobial agent.

In some embodiments, the antimicrobial agent is an antibacterial agent. In some embodiments, the antibacterial agent is an antibiotic. In some embodiments, the antibiotic is a bactericidal antibiotic. In some embodiments, the antibiotic is a bacteriostatic antibiotic. In some embodiments, the antibiotic is selected from an aminoglycoside antibiotic, a beta-lactam antibiotic, an ansamycin antibiotic, a macrolide antibiotic, a sulfonamide antibiotic, a quinolone antibiotic, an oxazolidinone antibiotic, and a glycopeptide antibiotic.

In some embodiments, the antibiotic is a beta-lactam selected from 2-(3-alanyl)clavam, 2-hydroxymethylclavam, 8-epi-thienamycin, acetyl-thienamycin, amoxicillin, amoxicillin sodium, amoxicillin trihydrate, amoxicillin-potassium clavulanate combination, ampicillin, ampicillin sodium, ampicillin trihydrate, ampicillin-sulbactam, apalcillin, aspoxicillin, azidocillin, azlocillin, aztreonam, bacampicillin, biapenem, carbenicillin, carbenicillin disodium, carfecillin, carindacillin, carpetimycin, cefacetril, cefaclor, cefadroxil, cefalexin, cefaloridine, cefalotin, cefamandole, cefamandole, cefapirin, cefatrizine, cefatrizine propylene glycol, cefazedone, cefazolin, cefbuperazone, cefcapene, cefcapene pivoxil hydrochloride, cefdinir, cefditoren, cefditoren pivoxil, cefepime, cefetamet, cefetamet pivoxil, cefixime, cefmenoxime, cefmetazole, cefminox, cefminox, cefmolexin, cefodizime, cefonicid, cefoperazone, ceforanide, cefoselis, cefotaxime, cefotetan, cefotiam, cefoxitin, cefozopran, cefpiramide, cefpirome, cefpodoxime, cefpodoxime proxetil, cefprozil, cefquinome, cefradine, cefroxadine, cefsulodin, ceftazidime, cefteram, cefteram pivoxil, ceftezole, ceftibuten, ceftizoxime, ceftriaxone, cefuroxime, cefuroxime axetil, cephalosporin, cephamycin, chitinovorin, ciclacillin, clavulanic acid, clometocillin, cloxacillin, cycloserine, deoxy pluracidomycin, dicloxacillin, dihydro pluracidomycin, epicillin, epithienamycin, ertapenem, faropenem, flomoxef, flucloxacillin, hetacillin, imipenem, lenampicillin, loracarbef, mecillinam, meropenem, metampicillin, meticillin, mezlocillin, moxalactam, nafcillin, northienamycin, oxacillin, panipenem, penamecillin, penicillin, phenethicillin, piperacillin, tazobactam, pivampicillin, pivcefalexin, pivmecillinam, pivmecillinam hydrochloride, pluracidomycin, propicillin, sarmoxicillin, sulbactam, sulbenicillin, talampicillin, temocillin, terconazole, thienamycin, and ticarcillin.

In some embodiments, the antibiotic is an aminoglycoside, selected from 1,2′-N-DL-isoseryl-3′,4′-dideoxykanamycin B, 1,2′-N-DL-isoseryl-kanamycin B, 1,2′-N[(S)-4-amino-2-hydroxybutyryl]-3′,4′-dideoxykanamycin B, 1,2′-N-[(S)-4-amino-2-hydroxybutyryq-kanamycin B, 1-N-(2-Aminobutanesulfonyl) kanamycin A, 1-N-(2-aminoethanesulfonyl)3,4′-dideoxyribostamycin, 1-N-(2-Aminoethanesulfonyl)3′-deoxyribostamycin, 1-N-(2-aminoethanesulfonyl)3′,4′-dideoxykanamycin B, 1-N-(2-aminoethanesulfonyl)kanamycin A, 1-N-(2-aminoethanesulfonyl)kanamycin B, 1-N-(2-aminoethanesulfonyl)rib stamycin, 1-N-(2-aminopropanesulfonyl)3′-deoxykanamycin B, 1-N-(2-aminopropanesulfonyl)3′,4′-dideoxykanamycin B, 1-N-(2-aminopropanesulfonyl)kanamycin A, 1-N-(2-aminopropanesulfonyl)kanamycin B, 1-N-(L-4-amino-2-hydroxy-butyryl)2,′3′-dideoxy-2′-fluorokanamycin A, 1-N-(L-4-amino-2-hydroxy-propionyl)2,′3′-dideoxy-2′-fluorokanamycin A, 1-N-DL-3′,4′-dideoxy-isoserylkanamycin B,1-N-DL-isoserylkanamycin, 1-N-DL-isoserylkanamycin B, 1—N[L-(−)-(alpha-hydroxy-gamma-aminobutyryl)]-XK-62-2,2′,3′-dideoxy-2′-fluorokanamycin A,2-hydroxygentamycin A3,2-hydroxygentamycin B, 2-hydroxygentamycin B1,2-hydroxygentamycin JI-20A, 2-hydroxygentamycin JI-20B, 3″-N-methyl-4″-C-methyl-3′,4′-dodeoxy kanamycin A, 3″-N-methyl-4″-C-methyl-3′,4′-dodeoxy kanamycin B, 3″-N-methyl-4″-C-methyl-3′,4′-dodeoxy-6′-methyl kanamycin B, 3 ‘,4’-Dideoxy-3′-eno-ribostamycin, 3′,4′-dideoxyneamine,3′,4′-dideoxyribostamycin, 3′-deoxy-6′-N-methyl-kanamycin B,3′-deoxyneamine,3′-deoxyribostamycin, 3′-oxysaccharocin,3,3′-nepotrehalosadiamine, 3-demethoxy-2″-N-formimidoylistamycin B disulfate tetrahydrate, 3-demethoxyistamycin B,3-O-demethyl-2-N-formimidoylistamycin B, 3-O-demethylistamycin B,3-trehalosamine,4″,6″-dideoxydibekacin, 4-N-glycyl-KA-6606VI, 5″-Amino-3′,4′,5″-trideoxy-butirosin A, 6″-deoxydibekacin,6′-epifortimicin A, 6-deoxy-neomycin (structure 6-deoxy-neomycin B),6-deoxy-neomycin B, 6-deoxy-neomycin C, 6-deoxy-paromomycin, acmimycin, AHB-3′,4′-dideoxyribostamycin,AHB-3′-deoxykanamycin B, AHB-3′-deoxyneamine, AHB-3′-deoxyribostamycin,AHB-4″-6″-dideoxydibekacin, AHB-6″-deoxydib ekacin, AHB-dideoxyneamine,AHB-kanamycin B, AHB-methyl-3′-deoxykanamycin B, amikacin, amikacin sulfate, apramycin, arbekacin, astromicin, astromicin sulfate, bekanamycin, bluensomycin, boholmycin, butirosin, butirosin B, catenulin, coumamidine gamma1, coumamidine gamma2,D,L-1-N-(alpha-hydroxy-beta-aminopropionyl) -XK-62-2, dactimicin,de-O-methyl-4-N-glycyl-KA-6606VI,de-O-methyl-KA-66061, de-O-methyl-KA-70381,destomycin A, destomycin B, di-N6′,03-demethylistamycin A, dibekacin, dibekacin sulfate, dihydrostreptomycin, dihydrostreptomycin sulfate, epi-formamidoylglycidylfortimicin B, epihygromycin, formimidoyl-istamycin A, formimidoyl-istamycin B, fortimicin B, fortimicin C, fortimicin D, fortimicin KE, fortimicin KF, fortimicin KG, fortimicin KG1 (stereoisomer KG1/KG2), fortimicin KG2(stereoisomer KG1/KG2), fortimicin KG3, framycetin, framycetin sulphate, gentamicin, gentamycin sulfate, globeomycin, hybrimycin A1, hybrimycin A2, hybrimycin B1, hybrimycin B2, hybrimycin C1, hybrimycin C2, hydroxystreptomycin, hygromycin, hygromycin B, isepamicin, isepamicin sulfate, istamycin, kanamycin, kanamycin sulphate, kasugamycin, lividomycin, marcomycin, micronomicin, micronomicin sulfate, mutamicin, myomycin, N-demethyl-7-O-demethylcelesticetin, demethylcelesticetin, methanesulfonic acid derivative of istamycin, nebramycin, nebramycin, neomycin, netilmicin, oligostatin, paromomycin, quintomycin, ribostamycin, saccharocin, seldomycin, sisomicin, sorbistin, spectinomycin, streptomycin, tobramycin, trehalosmaine, trestatin, validamycin, verdamycin, xylostasin, and zygomycin;

In some embodiments, the antibiotic is an ansa-type antibiotic selected from 21-hydroxy-25-demethyl-25-methylthioprotostreptovaricin, 3-methylthiorifamycin, ansamitocin, atropisostreptovaricin, awamycin, halomicin, maytansine, naphthomycin, rifabutin, rifamide, rifampicin, rifamycin, rifapentine, rifaximin, rubradirin, streptovaricin, and tolypomycin.

In some embodiments, the antibiotic is an anthraquinone selected from auramycin, cinerubin, ditrisarubicin, ditrisarubicin C, figaroic acid fragilomycin, minomycin, rabelomycin, rudolfomycin, and sulfurmycin.

In some embodiments, the antibiotic is an azole selected from azanidazole, bifonazole, butoconazol, chlormidazole, chlormidazole hydrochloride, cloconazole, cloconazole monohydrochloride, clotrimazol, dimetridazole, econazole, econazole nitrate, enilconazole, fenticonazole, fenticonazole nitrate, fezatione, fluconazole, flutrimazole, isoconazole, isoconazole nitrate, itraconazole, ketoconazole, lanoconazole, metronidazole, metronidazole benzoate, miconazole, miconazole nitrate, neticonazole, nimorazole, niridazole, omoconazol, ornidazole, oxiconazole, oxiconazole nitrate, propenidazole, secnidazol, sertaconazole, sertaconazole nitrate, sulconazole, sulconazole nitrate, tinidazole, tioconazole, and voriconazol.

In some embodiments, the antibiotic is a glycopeptide selected from acanthomycin, actaplanin, avoparcin, balhimycin, bleomycin B (copper bleomycin), chloroorienticin, chloropolysporin, demethylvancomycin, enduracidin, galacardin, guanidylfungin, hachimycin, demethylvancomycin, N-nonanoyl-teicoplanin, phleomycin, platomycin, ristocetin, staphylocidin, talisomycin, teicoplanin, vancomycin, victomycin, xylocandin, and zorbamycin.

In some embodiments, the antibiotic is a macrolide selected from acetylleucomycin, acetylkitasamycin, angolamycin, azithromycin, bafilomycin, brefeldin, carbomycin, chalcomycin, cirramycin, clarithromycin, concanamycin, deisovaleryl-niddamycin, demycinosyl-mycinamycin, Di-O-methyltiacumicidin, dirithromycin, erythromycin, erythromycin estolate, erythromycin ethyl succinate, erythromycin lactobionate, erythromycin stearate, flurithromycin, focusin, foromacidin, haterumalide, haterumalide, josamycin, josamycin ropionate, juvenimycin, juvenimycin, kitasamycin, ketotiacumicin, lankavacidin, lankavamycin, leucomycin, machecin, maridomycin, megalomicin, methylleucomycin, methymycin, midecamycin, miocamycin, mycaminosyltylactone, mycinomycin, neutramycin, niddamycin, nonactin, oleandomycin, phenylacetyldeltamycin, pamamycin, picromycin, rokitamycin, rosaramicin, roxithromycin, sedecamycin, shincomycin, spiramycin, swalpamycin, tacrolimus, telithromycin, tiacumicin, tilmicosin, treponemycin, troleandomycin, tylosin, and venturicidin.

In some embodiments, the antibiotic is a nucleoside selected from amicetin, angustmycin, azathymidine, blasticidin S, epiroprim, flucytosine, gougerotin, mildiomycin, nikkomycin, nucleocidin, oxanosine, oxanosine, puromycin, pyrazomycin, showdomycin, sinefungin, sparsogenin, spicamycin, tunicamycin, uracil polyoxin, and vengicide.

In some embodiments, the antibiotic is a peptide selected from actinomycin, aculeacin, alazopeptin, amfomycin, amythiamycin, antifungal from Zalerion arboricola, antrimycin, apid, apidaecin, aspartocin, auromomycin, bacileucin, bacillomycin, bacillopeptin, bacitracin, bagacidin, berninamycin, beta-alanyl-L-tyrosine, bottromycin, capreomycin, caspofungine, cepacidine, cerexin, cilofungin, circulin, colistin, cyclodepsipeptide, cytophagin, dactinomycin, daptomycin, decapeptide, desoxymulundocandin, echanomycin, echinocandin B, echinomycin, ecomycin, enniatin, etamycin, fabatin, ferrimycin, ferrimycin, ficellomycin, fluoronocathiacin, fusaricidin, gardimycin, gatavalin, globopeptin, glyphomycin, gramicidin, herbicolin, iomycin, iturin, iyomycin, izupeptin, janiemycin, janthinocin, jolipeptin, katanosin, killertoxin, lipopeptide antibiotic, lipopeptide from Zalerion sp., lysobactin, lysozyme, macromomycin, magainin, melittin, mersacidin, mikamycin, mureidomycin, mycoplanecin, mycosubtilin, neopeptifluorin, neoviridogrisein, netropsin, nisin, nocathiacin, nocathiacin 6-deoxyglycoside, nosiheptide, octapeptin, pacidamycin, pentadecapeptide, peptifluorin, permetin, phytoactin, phytostreptin, planothiocin, plusbacin, polcillin, polymyxin antibiotic complex, polymyxin B, polymyxin B1, polymyxin F, preneocarzinostatin, quinomycin, quinupristin-dalfopristin, safracin, salmycin, salmycin, salmycin, sandramycin, saramycetin, siomycin, sperabillin, sporamycin, a streptomyces compound, subtilin, teicoplanin aglycone, telomycin, thermothiocin, thiopeptin, thiostrepton, tridecaptin, tsushimycin, tuberactinomycin, tuberactinomycin, tyrothricin, valinomycin, viomycin, virginiamycin, and zervacin.

In some embodiments, the antibiotic is a polyene selected from amphotericin, amphotericin, aureofungin, ayfactin, azalomycin, blasticidin, candicidin, candicidin methyl ester, candimycin, candimycin methyl ester, chinopricin, filipin, flavofungin, fradicin, hamycin, hydropricin, levorin, lucensomycin, lucknomycin, mediocidin, mediocidin methyl ester, mepartricin, methylamphotericin, natamycin, niphimycin, nystatin, nystatin methyl ester, oxypricin, partricin, pentamycin, perimycin, pimaricin, primycin, proticin, rimocidin, sistomycosin, sorangicin, and trichomycin.

In some embodiments, the antibiotic is a polyether selected from 20-deoxy-epi-narasin, 20-deoxysalinomycin, carriomycin, dianemycin, dihydrolonomycin, etheromycin, ionomycin, iso-lasalocid, lasalocid, lenoremycin, lonomycin, lysocellin, monensin, narasin, oxolonomycin, a polycyclic ether antibiotic, and salinomycin.

In some embodiments, the antibiotic is a quinolone selected from alkyl-methylendioxy-4(1 H)-oxocinnoline-3-carboxylic acid, alatrofloxacin, cinoxacin, ciprofloxacin, ciprofloxacin hydrochloride, danofloxacin, dermofongin A, enoxacin, enrofloxacin, fleroxacin, flumequine, gatifloxacin, gemifloxacin, grepafloxacin, levofloxacin, lomefloxacin, lomefloxacin, hydrochloride, miloxacin, moxifloxacin, nadifloxacin, nalidixic acid, nifuroquine, norfloxacin, ofloxacin, orbifloxacin, oxolinic acid, pazufloxacine, pefloxacin, pefloxacin mesylate, pipemidic acid, piromidic acid, premafloxacin, rosoxacin, rufloxacin, sparfloxacin, temafloxacin, tosufloxacin, and trovafloxacin.

In some embodiments, the antibiotic is a steroid selected from aminosterol, ascosteroside, cladosporide, dihydrofusidic acid, dehydro-dihydrofusidic acid, dehydrofusidic acid, fusidic acid, and squalamine.

In some embodiments, the antibiotic is a sulfonamide selected from chloramine, dapsone, mafenide, phthalylsulfathiazole, succinylsulfathiazole, sulfabenzamide, sulfacetamide, sulfachlorpyridazine, sulfadiazine, sulfadiazine silver, sulfadicramide, sulfadimethoxine, sulfadoxine, sulfaguanidine, sulfalene, sulfamazone, sulfamerazine, sulfamethazine, sulfamethizole, sulfamethoxazole, sulfamethoxypyridazine, sulfamonomethoxine, sulfamoxol, sulfanilamide, sulfaperine, sulfaphenazol, sulfapyridine, sulfaquinoxaline, sulfasuccinamide, sulfathiazole, sulfathiourea, sulfatolamide, sulfatriazin, sulfisomidine, sulfisoxazole, sulfisoxazole acetyl, and sulfacarbamide.

In some embodiments, the antibiotic is a tetracycline selected from dihydrosteffimycin, demethyltetracycline, aclacinomycin, akrobomycin, baumycin, bromotetracycline, cetocyclin, chlortetracycline, clomocycline, daunorubicin, demeclocycline, doxorubicin, doxorubicin hydrochloride, doxycycline, lymecyclin, marcellomycin, meclocycline, meclocycline sulfosalicylate, methacycline, minocycline, minocycline hydrochloride, musettamycin, oxytetracycline, rhodirubin, rolitetracycline, rubomycin, serirubicin, steffimycin, and tetracycline.

In some embodiments, the antibiotic is a dicarboxylic acid selected from adipic acid, pimelic acid, suberic acid, azelaic acid, sebacic acid, 1,11-undecanedioic acid, 1,12-dodecanedioic acid, 1,13-tridecanedioic acid, and 1,14-tetradecanedioic acid.

In some embodiments, the antibiotic is an antibiotic metal or a metal ion, wherein the metal is selected from silver, copper, zinc, mercury, tin, lead, bismutin, cadmium, chromium, and gold.

In some embodiments, the antibiotic is a silver compound selected from silver acetate, silver benzoate, silver carbonate, silver iodate, silver iodide, silver lactate, silver laurate, silver nitrate, silver oxide, silver palmitate, silver protein, and silver sulfadiazine.

In some embodiments, the antibiotic is an oxidizing agent or a substance that releases free radicals or active oxygen, selected from oxygen, hydrogen peroxide, benzoyl peroxide, elemental halogen species, oxygenated halogen species, bleaching agents, perchlorite species, iodine, iodate, and benzoyl peroxide.

In some embodiments, the antibiotic is a cationic antimicrobial agent selected from quaternary ammonium compounds, alkyltrimethyl ammonium bromide, cetrimide, benzalkonium chloride, n-alkyldimethylbenzyl ammonium chloride, dialkylmethyl ammonium halide, and dialkylbenzyl ammonium halide;

In some embodiments, the antibiotic is a compound selected from chlorhexidine acetate, chlorhexidine gluconate and chlorhexidine hydrochloride, picloxydine, alexidine, polihexanide, chlorproguanil hydrochloride, proguanil hydrochloride, metformin hydrochloride, phenformin, and buformin hydrochloride.

In some embodiments, the antibiotic is an agent selected from abomycin, acetomycin, acetoxycycloheximide, acetylnanaomycin, an actinoplanessp. Compound, actinopyrone, aflastatin, albacarcin, albacarcin, albofungin, albofungin, alisamycin, alpha-R,S-methoxycarbonylbenzylmonate, altromycin, amicetin, amycin, amycin demanoyl compound, amycine, amycomycin, anandimycin, anisomycin, anthramycin, anti-syphilis imune substance, anti-tuberculosis immune substance, antibiotic from Eschericia coli, antibiotics from Streptomycesrefuineus, anticapsin, antimycin, aplasmomycin, aranorosin, aranorosinol, arugomycin, ascofuranone, ascomycin, ascosin, Aspergillus flavus antibiotic, asukamycin, aurantinin, an Aureolic acid antibiotic substance, aurodox, avilamycin, azidamfenicol, azidimycin, bacillaene, a Bacillus larvae antibiotic, bactobolin, benanomycin, benzanthrin, benzylmonate, bicozamycin, bravomicin, brodimoprim, butalactin, calcimycin, calvatic acid, candiplanecin, carumonam, carzinophilin, celesticetin, cepacin, cerulenin, cervinomycin, chartreusin, chloramphenicol, chloramphenicol palmitate, chloramphenicol succinate sodium, chlorflavonin, chlorobiocin, chlorocarcin, chromomycin, ciclopirox, ciclopirox olamine, citreamicin, cladosporin, clazamycin, clecarmycin, clindamycin, coliformin, collinomycin, copiamycin, corallopyronin, corynecandin, coumermycin, culpin, cuprimyxin, cyclamidomycin, cycloheximide, dactylomycin, danomycin, danubomycin, delaminomycin, demethoxyrapamycin, demethylscytophycin, dermadin, desdamethine, dexylosyl-benanomycin, pseudoaglycone, dihydromocimycin, dihydronancimycin, diumycin, dnacin, dorrigocin, dynemycin, dynemycin triacetate, ecteinascidin, efrotomycin, endomycin, ensanchomycin, equisetin, ericamycin, esperamicin, ethylmonate, everninomicin, feldamycin, flambamycin, flavensomycin, florfenicol, fluvomycin, fosfomycin, fosfonochlorin, fredericamycin, frenolicin, fumagillin, fumifungin, funginon, fusacandin, fusafungin, gelbecidine, glidobactin, grahamimycin, granaticin, griseofulvin, griseoviridin, grisonomycin, hayumicin, hayumicin, hazymicin, hedamycin, heneicomycin, heptelicid acid, holomycin, humidin, isohematinic acid, karnatakin, kazusamycin, kristenin, L-dihydrophenylalanine, a L-isoleucyl-L-2-amino-4-(4′-amino-2′,5′-cyclohexadienyl) derivative, lanomycin, leinamycin, leptomycin, libanomycin, lincomycin, lomofungin, lysolipin, magnesidin, manumycin, melanomycin, methoxycarbonylmethylmonate, methoxycarbonylethylmonate, methoxycarbonylphenylmonate, methyl pseudomonate, methylmonate, microcin, mitomalcin, mocimycin, moenomycin, monoacetyl cladosporin, monomethyl cladosporin, mupirocin, mupirocin calcium, mycobacidin, myriocin, myxopyronin, pseudoaglycone, nanaomycin, nancimycin, nargenicin, neocarcinostatin, neoenactin, neothramycin, nifurtoinol, nocardicin, nogalamycin, novobiocin, octylmonate, olivomycin, orthosomycin, oudemansin, oxirapentyn, oxoglaucine methiodide, pactacin, pactamycin, papulacandin, paulomycin, phaeoramularia fungicide, phenelfamycin, phenyl, cerulenin, phenylmonate, pholipomycin, pirlimycin, pleuromutilin, a polylactone derivative, polynitroxin, polyoxin, porfiromycin, pradimicin, prenomycin, Prop-2-enylmonate, protomycin, Pseudomonas antibiotic, pseudomonic acid, purpuromycin, pyrinodemin, pyrrolnitrin, pyrrolomycin, amino, chloro pentenedioic acid, rapamycin, rebeccamycin, resistomycin, reuterin, reveromycin, rhizocticin, roridin, rubiflavin, naphthyridinomycin, saframycin, saphenamycin, sarkomycin, sarkomycin, sclopularin, selenomycin, siccanin, spartanamicin, spectinomycin, spongistatin, stravidin, streptolydigin, streptomycesarenae antibiotic complex, streptonigrin, streptothricins, streptovitacin, streptozotocine, a strobilurin derivative, stub omycin, sulfamethoxazol-trimethoprim, sakamycin, tejeramycin, terpentecin, tetrocarcin, thermorubin, thermozymocidin, thiamphenicol, thioaurin, thiolutin, thiomarinol, thiomarinol, tirandamycin, tolytoxin, trichodermin, trienomycin, trimethoprim, trioxacarcin, tyrissamycin, umbrinomycin, unphenelfamycin, urauchimycin, usnic acid, uredolysin, variotin, vermisporin, verrucarin, and analogs, salts and derivatives thereof.

In some embodiments, the antibiotic is selected from the group of aminoglycoside, ansamycin, carbacephem, carbapenem, cephalosporin, fosfomycin, glycopeptide, lincosamide, lipopeptide, macrolide, monobactam, nitrofuran, oxazolidinone, penicillin, quinolone, sulfonamide, and tetracycline.

In some embodiments, at least 1, 2, 3, 4, or 5 or more antimicrobial agents are selected from the group of aminoglycoside, ansamycin, carbacephem, carbapenem, cephalosporin, fosfomycin, glycopeptide, lincosamide, lipopeptide, macrolide, monobactam, nitrofuran, oxazolidinone, penicillin, quinolone, sulfonamide, and tetracycline.

In some embodiments, at least one antimicrobial agent is cephalosporin. In some embodiments, the cephalosporin is selected from the group of first generation cephalosporin, second generation cephalosporin, third generation cephalosporin, fourth generation cephalosporin, and fifth generation cephalosporin.

In some embodiments, at least one antimicrobial agent is quinolone. In some embodiments, quinolone is a fluoroquinolone.

In some embodiments, at least 1, 2, 3, 4, 5, 6, 7, 8, 9, 10, 11, 12, 13, 14, or 15 or more antimicrobial agents are selected from the group of gentamicin, ciprofloxacin, cefazolin, ceftriaxone, cefepime, ampicillin, imipenem, trimethoprim, sulfamethoxazole, amikacin, nitrofurantoin, fosfomycin, piperacillin, tazobactam, amoxicillin, and clavulanate.

In some embodiments, the antibiotic is selected from the group of gentamicin, ciprofloxacin, cefazolin, ceftriaxone, cefepime, ampicillin, imipenem, trimethoprim, sulfamethoxazole, amikacin, nitrofurantoin, fosfomycin, piperacillin, tazobactam, amoxicillin, and clavulanate.

In some embodiments, the at least one antimicrobial agent includes a beta-lactamase inhibitor. In some embodiments, the beta-lactamase inhibitor is selected from clavulanate, sulbactam, tazobactam, avibactam, relebactam, tebipenem, y-methylidene Penem, and boron based transition state inhibitors. In some embodiments, the beta-lactamase inhibitor is accompanied by a beta-lactam antibiotic.

The disclosure illustratively described herein can suitably be practiced in the absence of any element or elements, limitation or limitations, not specifically disclosed herein. Thus, for example, the terms “comprising”, “including,” containing“, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the disclosure claimed.

While this invention has been described with reference to illustrative embodiments and examples, the description is not intended to be construed in a limiting sense. Thus, various modifications of the illustrative embodiments, as well as other embodiments of the invention, will be apparent to persons skilled in the art upon reference to this description. It is therefore contemplated that the appended claims will cover any such modifications or embodiments.

All publications, patents and patent applications referred to herein are incorporated by reference in their entirety to the same extent as if each individual publication, patent or patent application was specifically and individually indicated to be incorporated by reference in its entirety.

What is claimed is:

1. A method for determining susceptibility of bacteria in a clinical sample to an antibiotic agent, the method comprising:
a) inoculating a test amount of the clinical sample in a medium containing a predetermined rate-targeted concentration of the antibiotic agent that is equal to or above a pre-determined minimal inhibitory concentration (MIC) cutoff for the clinical sample and the antibiotic agent;
b) inoculating a control amount of the clinical sample in a medium that does not contain the antibiotic agent, wherein the control amount is the same as the test amount;
c) incubating the test amount for an incubation period;
d) incubating the control amount for the incubation period;
e) determining a quantity of RNA in the test amount and a quantity of RNA in the control amount at a conclusion of the incubation period that is less than 480 minutes after the completion of step a); and
f) determining a susceptibility of the bacteria to the antibiotic agent by comparing the quantity of RNA in the test amount to the quantity of the RNA in the control amount, wherein the bacteria is considered susceptible to the antibiotic agent when the quantity of RNA in the test amount less than the quantity of RNA in the control amount at the conclusion of the incubation period.

2. The method of claim 1, wherein incubating the test amount is done within a test incubation chamber on a centrifugal disc, and incubating the control amount is done within a control incubation chamber on the same centrifugal disc.

3. The method of claim 2, wherein the test incubation chamber is fluidically isolated from the control incubation chamber.

4. The method of claim 1, wherein the RNA comprises at least one of pre-ribosomal RNA, mature RNA, ribosomal RNA, 16S rRNA, and 23S rRNA.

5. The method of claim 1, wherein the incubation period is equal to or less than 450 minutes.

6. The method of claim 1, wherein the antibiotic agent comprises at least one of Gentamicin, Ciprofloxacin, Cefazolin, Ceftriaxone, Cefepime, Ampicillin, Trimethoprim-Sulfamethoxazole, Nitrofurantoin, Fosfomycin, Amoxicillin-Clavulanate, Amikacin, Ertapenem, Meropenem, and combinations thereof.

7. The method of claim 1, wherein the bacteria is an unknown bacteria when steps a) to f) of claim 1 are conducted.

8. The method of claim 1, further comprising lysing the test amount prior to determining the quantity of RNA in the test amount and lysing the control amount prior to determining the quantity of RNA in the control amount.

9. The method of claim 8, wherein lysing the test amount comprises:
g) subjecting the test amount to mechanical lysis to cause disruption of a cellular membrane in the bacteria;
h) contacting the test amount with an alkaline material to produce a lysate composition comprising the RNA; and
i) recovering the lysate composition from the test amount.

10. The method of claim 9, wherein Step h) comprises contacting the bacteria in the test amount with an alkaline material.

11. The method of claim 8, wherein incubating the test amount is done within a test incubation chamber on a centrifugal disc, and lysing the test amount is conducted within a lysing chamber on the same centrifugal disc.

12. The method of claim 9, wherein Step h) is carried out after commencement of disruption of the cellular membrane in Step g).

13. The method of claim 1, wherein the bacteria is susceptible to the antibiotic agent when the quantity of RNA in the test amount is about 40% or less of the quantity of RNA in the control amount at the conclusion of the incubation period.

14. The method of claim 1, wherein the clinical sample comprises mammalian cellular material.

15. The method of claim 1, wherein the clinical sample comprises a bodily fluid selected from the group consisting of blood, urine, saliva, sweat, tears, mucus, breast milk, plasma, serum, synovial fluid, pleural fluid, lymph fluid, amniotic fluid, feces, cerebrospinal fluid, and any mixture of two or more of these.

16. The method of claim 1, wherein the predetermined rate-targeted concentration is equal to or above the resistant Clinical and Laboratory Standards Institute (CLSI) MIC cutoff for urine for the antibiotic agent.

17. The method of claim 16, wherein the predetermined rate-targeted concentration is at least 2-fold or greater than the resistant CLSI MIC cutoff for urine for the clinical sample and the antibiotic agent.

18. The method of claim 1, wherein the antibiotic agent and the rate-targeted concentration is selected from:

| Antibiotic | Rate-targeted concentration (μg/mL) |
|---|---|
| Amikacin | 64-2 |
| Amoxicillin/Clavulanate | 256-1 |
| Ampicillin | 2048-8 |
| Cefazolin | 256-2 |
| Cefepime | 128-4 |
| Ceftriaxone | 128-1 |
| Ciprofloxacin | 8-1 |
| Ertapenem | 8-0.5 |
| Fosfomycin | 512-4 |
| Gentamicin | 16-2 |
| Meropenem | 8-1 |
| Nitrofurantoin | 512-4 |
| Trimethoprim/sulfamethoxazole | 304-2 |

19. The method of claim 18, wherein the incubation period is 90-120 minutes and the antibiotic agent and the rate-targeted concentration is selected from:

| Antibiotic | Rate-targeted concentration (μg/mL) |
|---|---|
| Amikacin | 32-8 |
| Amoxicillin/Clavulanate | 64-8 |
| Ampicillin | 128 |
| Cefazolin | 64 |
| Cefepime | 64-32 |
| Ceftriaxone | 32 |
| Ciprofloxacin | 4 |
| Ertapenem | 4-2 |
| Fosfomycin | 64 |
| Gentamicin | 4-2 |
| Meropenem | 4-2 |
| Nitrofurantoin | 16 |
| Trimethoprim/sulfamethoxazole | 76-4 |
